(19) United States
(12) Patent Application Publication
(10) Pub. No.: US 2006/0228364 A1
(43) Pub. Date: Oct. 12, 2006

(54) Serum albumin binding peptides for tumor targeting

(75) Inventors: Mark S. Dennis, San Carlos, CA (US); Henry B. Lowman, El Granada, CA (US); Warren L. DeLano, San Carlos, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/367,100

(22) Filed: Mar. 2, 2006

Related U.S. Application Data
(63) Continuation-in-part of application No. 11/106,415, filed on Apr. 13, 2005.
Application No. 11/106,415 is a continuation-in-part of application No. 10/186,229, filed on Jun. 28, 2002.
(63) Continuation-in-part of application No. 10/149,835, filed on Jun. 14, 2002.
(60) Provisional application No. 60/173,048, filed on Dec. 24, 1999.

(51) Int. Cl. A61K 39/395

(52) U.S. Cl. 424/155.1; 424/178.1

(57) ABSTRACT

Peptide ligands having affinity for serum albumin are useful for tumor targeting. Conjugate molecules comprising a serum albumin binding peptide fused to a biologically active molecule demonstrate modified pharmacokinetic properties as compared with the biologically active molecule alone, including tissue (e.g., tumor) uptake, infiltration, and diffusion.

This application is a continuation-in-part application claiming priority to U.S. application Ser. No. 11/106,415, filed Apr. 13, 2005, which is a continuation-in-part application claiming priority to U.S. application Ser. No. 10/186,229, filed Jun. 28, 2002, and this application is a continuation-in-part claiming priority to U.S. application Ser. No. 10/149,835, filed Jun. 14, 2002, which is the U.S. National Stage of International Application No. PCT/US00/35325, filed Dec. 22, 2000, which claims benefit of U.S. Provisional Application No. 60/173,048, filed Dec. 24, 1999, the entire disclosures of which are herein incorporated by reference.

### FIELD OF THE INVENTION

This invention relates to compounds comprising a peptide ligand domain and an active domain, useful, for example, as therapeutic and diagnostic agents. In particular, hybrid molecules comprising a peptide ligand domain that binds serum albumin and a active domain, such as a biologically active molecule, are useful as tumor targeting agents, having altered pharmacokinetic and pharmacological properties as compared to the active domain alone.

### DESCRIPTION OF RELATED DISCLOSURES

Therapeutic methods for the treatment of disease rely on the administration of a therapeutic molecule to a patient, the distribution of the administered therapeutic in the body, generally via blood circulation, and the uptake and efficacy of the administered drug at the target tissue. The effectiveness of an administered protein depends heavily on upon the intrinsic pharmacokinetics of the molecule, for example, protein. Generally, high doses are utilized to offset rapid and efficient clearance of such molecules, for example, protein therapeutics from the circulation, including degradation mechanisms. As a consequence, the amount of time that the therapeutic molecule is exposed to the desired tissue may be short, reducing possible therapeutic effects.

Several parameters can be addressed to improve efficacy and efficiency of an administered therapeutic molecule. These include increasing half-life, increasing uptake into tissue, and increasing diffusion of the molecule into tissue. Decreasing the size of the molecule, for example, administering a Fab fragment rather than a full-size IgG molecule, improves two of these parameters, tissue uptake and diffusion. However, decreased size is also associated with more rapid clearance and reduced half-life. See, for example, Adams et. al., 1999, J. Immunol. Methods 231:249-260. For most applications, these parameters must be balanced, so that optimization of one factor does not lead to difficulties with another.

For the treatment of tumors, several approaches have been suggested to increase half-life of therapeutic molecules. Because the kidney generally filters out molecules below 60 kDa, efforts to reduce clearance have generally focused on increasing molecular size through protein fusions, glycosylation, or the addition of polyethylene glycol polymers (i.e., PEG). For example, small therapeutic molecules have been fused to large, long-lived proteins such as albumin (Syed et. al., 1997, Blood 89:3243-3252; Yeh et. al., 1992, PNAS USA 89:1904-1908), or the Fc portion of an IgG (Ashkenazi et. al., 1997, Curr. Opin. in Immunol. 9:195-200). Glycosylation sites have been introduced to the molecules (Keyt et. al., 1994, PNAS USA 91:3670-74), and molecules have been conjugated with PEG (Clark et. al., 1996, J. Biol. Chem., 271: 21969-77; Lee et. al, 1999, Bioconjugate Chem. 10:973-981; Tanaka et. al., 1991, Cancer Res. 51:3710-14) to increase size, and thereby increase elimination half-times. Through these methods, the in vivo exposure of protein therapeutics has been extended.

A serum albumin-CD4 conjugate in which the V1 and V2 domains of CD4 were fused with human serum albumin (HSA) has been described (Yeh, et al., 1992, Proc. Natl. Acad. Sci. USA 89:1904-1908). The conjugate's elimination half-time was 140-fold that of a soluble CD4 (sCD4) in a rabbit experimental model.

Extended in vivo half-times of human soluble complement receptor type 1 (sCR1) fused to the albumin binding domains from Streptococcal protein G have been reported (Makrides et al. 1996 J. Pharmacol. Exptl. Ther. 277:532-541). The constructs contained albumin binding domains of protein G having approximately 80 amino acids (fragment BA), and approximately 155 amino acids (fragment BABA).

The pharmacokinetics of a labeled IgG binding domain derived from the Z domain of protein A having approximately 60 amino acids and of a serum albumin binding domain derived from Streptococcal protein G (B-domain) having approximately 200 amino acids have been described (EP 0 486,525).

The binding of therapeutic agents to serum albumin has been suggested to alter pharmacodynamics in specific situations. For example, it has been suggested that the pharmacodynamics of insulin are altered if bound to serum albumin. Acylation of insulin with saturated fatty acids containing 10-16 carbon atoms produces insulin with affinity for albumin (Kurtzhals et al. 1995 Biochem. J. 312:725-731). Differences in albumin binding affinity among acylated insulins were correlated with the timing of the blood-glucose lowering effects of the various molecules after subcutaneous injection into rabbits. Tighter binding to albumin was correlated with a delay in blood glucose lowering, possibly due to acylated insulin binding albumin in the subcutaneous tissue, resulting in a lower absorption rate of the acylated insulins when compared with non-acylated insulin.

Covalent fusion of the therapeutic compound, methotrexate to human serum albumin was reported to improve plasma half-life, tumor accumulation, and uptake of methotrexate (Burger et. al., 2001 Int. J. Cancer 92:718-724).

Small molecule drugs have utilized association with albumin to improve pharmacokinetic properties in vivo, however, drugs associated with plasma protein are usually unavailable for binding to a target, despite an extended half-life. Because only the unbound fraction is generally functionally active, a fine balance must be maintained between the concentration of free drug required for efficacy and the frequency at which the drug must be administered (Rowland M., ed., 1988, In: Clinical Pharmacokinetics: Concepts and Applications, 2d Ed, Lea & Febigen, Philadelphia.

Conjugation of therapeutic molecules to serum proteins such as albumin, thus is not generally considered suitable for efficient clinical use, particularly for conjugation to intact immunoglobulins. While an increase in size by binding albumin may be expected to extend the exposure of molecules in vivo, the large size and association with albumin would be expected to hinder free molecule diffusion into tissue, particularly tumor uptake and distribution. In addition, such large molecules are inefficient to produce and administer.

New compositions and methods providing protein therapeutics to tissue, such as tumor cells, are needed, particularly those that maximize tissue (e.g., tumor) exposure, uptake, and diffusion of the therapeutic protein in the tissue (tumor). Such compositions and methods are needed to enhance therapeutic efficacy and reduce side effects associated with some protein therapies.

Phage-display techniques were used to identify novel peptide binding ligands that bind specifically to plasma proteins, such as serum albumin. Hybrid molecules containing the peptide binding ligands (peptide binding domain) and a biologically active molecule (active domain) were found to have prolonged elimination half-times as compared with the active domain alone. See, for example, WO01/45746, published 28 Jun. 2001, the contents of which are hereby incorporated by reference for all purposes. It has now been discovered that serum albumin binding peptides can alter the pharmacodynamics of fused active domain molecules, including alteration of tissue uptake, penetration, and diffusion. Moreover, these parameters can be modulated by specific selection of the appropriate serum binding peptide.

### SUMMARY OF THE INVENTION

The present invention provides conjugate molecules having a peptide ligand domain and an active domain. The conjugate molecules provide for altered pharmacodynamics of the active domain molecule, including alteration of tissue uptake, penetration, and diffusion. In a preferred embodiment, a hybrid molecule comprises a serum albumin binding peptide fused to a therapeutic protein, having improved tumor targeting, tumor penetration, diffusion within the tumor, and enhanced efficacy as compared with the therapeutic protein alone. In one embodiment, therapeutic methods effectively and efficiently utilize a reduced amount of the fused therapeutic ligand, resulting in reduced side effects, such as reduced non-tumor cell cytotoxicity. In another embodiment, the peptide binding ligand is selected to alter the rate of tissue uptake and penetration of a fused therapeutic ligand, for example, to match the rate of internalization of the ligand's receptors in the tissue for maximal therapeutic efficacy.

The present invention utilizes compounds that bind to serum albumin. The compounds of the present invention (referred to as peptide ligands) are, for example, peptides or peptide derivatives such as peptide mimetics and peptide analogs. According to preferred aspects of the invention, the compounds are non-naturally occurring amino acid sequences that bind plasma proteins such as serum albumin. Preferably the peptide ligand is a non-naturally occurring amino acid sequence of between about 10 and 20 amino acid residues.

Such compounds preferably bind a serum albumin with an affinity characterized by a dissociation constant, K_{d}, that is less than about 100 μM, preferably less than about 100 nM, and preferably do not substantially bind other plasma proteins. Specific examples of such compounds include linear or cyclic, especially cyclic peptides, preferably between about 10 and 20 amino acid residues in length, and combinations thereof, optionally modified at the N-terminus or C-terminus or both, as well as their salts and derivatives, functional analogues thereof and extended peptide chains carrying amino acids or polypeptides at the termini of the sequences.

Preferred peptide ligands that bind serum albumin include linear and cyclic peptides, preferably cyclic peptide compounds comprising the following formulae:

[SEQ ID NO: 1]

Xaa-Xaa-Cys-Xaa-Xaa-Xaa-Xaa-Xaa-Cys-Xaa-Xaa
Phe-Cys-Xaa-Asp-Trp-Pro-Xaa-Xaa-Xaa-Ser-Cys

[SEQ ID NO: 2]

Val-Cys-Tyr-Xaa-Xaa-Xaa-Ile-Cys-Phe

[SEQ ID NO: 3]

Cys-Tyr-Xaa₁-Pro-Gly-Xaa-Cys
and

[SEQ ID NO: 4]

Asp-Xaa-Cys-Leu-Pro-Xaa-Trp-Gly-Cys-Leu-Trp

Preferred are peptide compounds of the general formulae comprising additional amino acids at the N-terminus (Xaa)ₓ and additional amino acids at the C-terminus (Xaa)_{z}, wherein Xaa is an amino acid and x and z are a whole number greater or equal to 0 (zero), generally less than 100, preferably less than 10 and more preferably 0, 1, 2, 3, 4 or 5 and more preferably 4 or 5 and Xaa₁ is selected from the group consisting of Ile, Phe, Tyr, and Val. In one embodiment, the invention relates to the use of an albumin binding peptide that includes the core sequence: DICLPRWGCLW [SEQ ID NO: 8], that binds albumin with high affinity and with a 1:1 stochiometry at a site that is distinct from known, small molecule albumin binding sites.

In particular aspects the invention is directed to combinations of a peptide ligand with a bioactive compound to form a hybrid molecule that comprises a peptide ligand domain and an active domain. The bioactive compounds of the invention include any compound useful as a therapeutic or diagnostic agent. Non-limiting examples of bioactive compounds include polypeptides such as enzymes, hormones, cytokines, antibodies, or antibody fragments, as well as organic compounds such as analgesics, antipyretics, antiinflammatory agents, antibiotics, antiviral agents, anti-fungal drugs, cardiovascular drugs, drugs that affect renal function and electrolyte metabolism, drugs that act on the central nervous system, and chemotherapeutic drugs, to name but a few.

In preferred embodiments, the bioactive compound is a protein, preferably a therapeutic protein such as a therapeutic antibody, including antigen binding antibody fragments. Examples include anti-HER2, anti-CD20, anti-VEGF, anti-EGFR, and other therapeutic antibodies. Most preferred are antibodies or antibody fragments that bind antigens expressed on pathogenic cells, such as tumor cells expressing HER2.

In preferred embodiments, the hybrid molecules comprising a peptide ligand domain and an active domain have improved pharmacokinetic or pharmacodynamic properties as compared to the same bioactive molecule comprising the active domain but lacking the peptide ligand domain. Such improved properties permit low-dose pharmaceutical formulations and novel pharmaceutical compositions, as well as targeted delivery to tissues and cells at an appropriate physiological rate, for example, to match rates of receptor internalization . The invention provides for methods of using the hybrid molecules in therapeutic and diagnostic methods, for example for tumor targeting therapeutics having an altered rate of uptake or tissue diffusion as compared with the active domain alone.

In particular aspects, the invention is directed to combinations of peptide ligands with bioactive compounds that have relatively short elimination half-times. The combinations are prepared with various objectives in mind, including improving the therapeutic or diagnostic efficacy of the bioactive compound in aspects of the invention involving in vivo use of the bioactive compound, by for example, modulating the tissue penetration and diffusion of the bioactive compound. For example, uptake and tissue, eg., tumor penetration of a bioactive compound can be modulated, e.g., enhanced, by fusing or linking (i.e., “conjugating”) a serum albumin binding peptide to the a bioactive compound. The choice of peptide, having a desired affinity for albumin and /or rate of tissue penetration, can provide tailored administration to optimize efficacy. Such combinations or fusions are conveniently made in recombinant host cells, or by the use of bifunctional crosslinking agents.

The present invention further extends to therapeutic and diagnostic applications for the compositions described herein. Therefore, the invention includes pharmaceutical compositions comprising a pharmaceutically acceptable excipient and the hybrid molecules of the invention.

### BRIEF DESCRIPTION OF THE DRAWINGS

FIG. 1 is photograph showing the binding of clones from the RB soft radomization library to different species of albumin immobilized on microtitre wells. Clones failed to bind ovalbumin or casein.

FIG. 2 is a bargraph showing amino acid preferences for binding rabbit albumin following full randomization with selection on rabbit albumin for the library:

| | |
|---|---|
| X₅DXCLPXWGCLWX4. | [SEQ ID NO: 155] |

FIG. 3 is a graph showing results of a competition assay demonstrating inhibition of RD and BA phage binding to rat or rabbit albumin in the presence of the peptide SA08. RD (open circles); BA (filled circles); HA (filled squares); HB (open squares)

FIG. 4 is a schematic diagram showing a serum albumin binding peptide sequence fused to the carboxy terminus of the light chain (D3H44-L) or heavy chain (D3H44-Ls) of Fab, and in identical constructs having the intra-chain disulfide replaced by alanines (D3H44-Ls and D3H44-Hs, respectively).

FIG. 5 is a graph demonstrating D3H44 fusions retained their ability to bind TF as measured using a FX activation assay.

FIG. 6 is a graph demonstrating D3H44 fusions retained their ability to bind TF as measured using a prothrombin time assay that measures prolongation of tissue factor dependent clotting.

FIG. 7 is a graph demonstrating that, unlike D3H44 lacking the albumin binding sequence (WT), both D3H44-L and D3H44-Ls bind to albumin as measured in the SA08b binding assay.

FIG. 8 is a graph demonstrating both D3H44 albumin-binding fusions bind TF and albumin simultaneously as judged by a biotin-TF binding assay.

FIG. 9 is a graph demonstrating fusion of the albumin binding peptide to D3H44 results in a protein having improved pharmacokinetic parameters.

FIG. 10 is a graph showing binding of the anti-HER2 antibody Fab fragment (Fab4D5), a fusion of Fab4D5 and serum albumin binding peptide (4D5H), and diabody (dia4D5) to albumin, as measured in the SA08b competition ELISA.

FIG. 11 is a graph demonstrating the fusion 4D5-H inhibits Herceptin® antibody binding to immobilized antigen, HER2, in the presence or absence of albumin.

FIG. 12 shows a schematic representation of the simultaneous binding of a Fab4D5-serum albumin binding peptide fusion (4D5-H) or diabody (dia 4D5-H) to immobilized albumin and biotin-labeled HER2 and a a graph showing detection of 4D5-H or dia 4D5-H immobilized on albumin and simultaneously bound by biotinlyated HER2.

FIG. 13 is a graph showing a time course of normalized plasma concentration for the fusion 4D5-H compared with that of the Fab 4D5 after administration to nude mice bearing HER2+ tumors.

FIG. 14 is a photograph showing uptake and diffusion of CY3-labeled fusion peptide (4D5-H) as compared with CY3-labeled Fab (4D5) and CY3-labeled IgG (Herceptin) over time (2, 24, and 48 hours post in vivo injection).

FIG. 15 is a photograph showing an SDS-PAGE analysis of the AB.Fab variants described in Table 12. Lane (1) AB.Fab4D5-H, (2) AB.Fab4D5-H4, (3) AB.Fab4D5-H8, (4) AB.Fab4D5-H10 and (5) AB.Fab4D5-H11 under oxidized and reduced conditions.

FIGS. 16A-16B are graphical representations of soluble albumin binding ELISA parameters. FIG. 16A: Incubation times of 1 (filled diamond), 2 (filled square) and 16 (filled triangle) hours were examined to determine the time required for equilibrium between AB.Fab4D5-H and soluble rabbit albumin to be reached. The dissociation constants (Kd) were 30, 42, and 64 nM, respectively. (B) The optimum time required to capture free AB.Fab4D5-H on plates coated with rabbit albumin was investigated over 15 (filled diamonds), 30 (filled squares), 45 (filled triangle), 60 (X) and 120 (filled circle) minutes. The dissociation constants were 33, 33, 29, 24, and 24 nM, respectively. “V” refers to the fraction of Bound AB.Fab and “a” refers to the concentration of free albumin.

FIGS. 17A-17C are graphical representations of pharmacokinetic profiles of AB.Fab variants in mouse, rat and rabbit. Fab4D5 (filled circle), AB.Fab4D5-H (circle), AB.Fab4D5-H4 (square), AB.Fab4D5-H8 (diamond), AB.Fab4D5-H10 (triangle) and AB.Fab4D5-H11 (inverted triangle) were dosed at (A) 5mg/kg, IV bolus in mice (9 mice/group, 3 mice/timepoint) (B) 5 mg/kg, IV bolus in rats (4 rats/group) and (C) 0.5 mg/kg, IV bolus in NZW rabbits (3 rabbits/group). Samples taken at the indicated times were assayed using a HER2 Binding ELISA.

FIGS. 18A-18B are graphical representations of albumin binding affinity vs. clearance or beta half-life in rats and rabbits. The affinity of AB.Fab4D5-H (circle), AB.Fab4D5-H4 (square), AB.Fab4D5-H8 (diamond), AB.Fab4D5-H10 (triangle) and AB.Fab4D5-H11 (inverted triangle) for rabbit (filled symbols) and rat (open symbols) albumin are plotted against their clearance (FIG. 18A) and beta half-life (FIG. 18B) observed in vivo. The data was fit for rabbit (solid line) and rat (dashed line) using a power function curve fit for clearance and a logarithmic curve fit for beta half-life.

FIGS. 19A-19B are graphical representations of allometric scaling to estimate the clearance and beta half-life of an AB.Fab in human having an affinity for human serum albumin of 500 nM. The clearance (FIG. 19A) and beta half-life (FIG. 19B) of AB.Fab4D5-H4 (filled square) in rabbits and AB.Fab4D5-H10 (open triangle) in rats is plotted as a function of body weight. The data was extrapolated to human (70 kg) suggesting a clearance of 76 ml/h and a beta half-life of 4 days for an AB.Fab having an affinity for human serum albumin of 500 nM.

FIG. 20 shows photographs of HER2-expression breast tumor cells stained with FITC-conjugated Herceptin®Fab4D5 and AB.Fab4D5-H (green spots). Cell nuclei are stained blue with DAPI and vasculature is stained red with a Cy3-conjugated anti-CD3 1 antibody.

FIGS. 21A-21D show bar graphs resulting from the quantitative analysis and comparison of tumor tissue penetration by Herceptin®, Fab and Ab.Fab as described in Example 6.

### DETAILED DESCRIPTION OF THE PREFERRED EMBODIMENTS

I. Definitions

The term “peptide ligand” within the context of the present invention is meant to refer to non-naturally occurring amino acid sequences that function to bind a particular target molecule. Peptide ligands within the context of the present invention are generally constrained (that is, having some element of structure as, for example, the presence of amino acids which initiate a beta-turn or beta- pleated sheet, or for example, cyclized by the presence of disulfide-bonded Cys residues) or unconstrained (linear) amino acid sequences of less than about 50 amino acid residues, and preferably less than about 40 amino acids residues. Of the peptide ligands less than about 40 amino acid residues, preferred are the peptide ligands of between about 10 and about 30 amino acid residues and especially the peptide ligands of about 20 amino acid residues. However, upon reading the instant disclosure, the skilled artisan will recognize that it is not the length of a particular peptide ligand but its ability to bind a particular target molecule that distinguishes the peptide ligand of the present invention. Therefore peptide ligands of 3, 4, 5, 6, 7, 8, 9, 10, 11, 12, 13, 14, 15, 16, 17, 18, 19, 20, 21, 22, 23, 24, and 25 amino acid residues, for example, are equally likely to be peptide ligands within the context of the present invention.

A peptide ligand of the present invention will bind a target molecule with sufficient affinity and specificity if the peptide ligand “homes” to, “binds” or “targets” a target molecule such as a specific cell type bearing the target molecule in vitro and preferably in vivo (see, for example, the use of the term “homes to,” “homing,” and “targets” in Pasqualini and Ruoslahti, 1996 Nature, 380:364-366 and Arap et al., 1998 Science, 279:377-380). In general, the peptide ligand will bind a target molecule with an affinity characterized by a dissociation constant, Kd, of less than about 1 μM, preferably less than about 100 nM and more preferably less than about 10 nM. However, peptide ligands having an affinity for a target molecule of less than about 1 nM and preferably between about 1 pM and 1 nM are equally likely to be peptide ligands within the context of the present invention. In general, a peptide ligand that binds a particular target molecule as described above can be isolated and identified by any of a number of art-standard techniques as described herein.

Peptides ligands are amino acid sequences as described above that may contain naturally as well as non-naturally occurring amino acid residues. Therefore, so-called “peptide mimetics” and “peptide analogs”, that may include non-amino acid chemical structures that mimic the structure of a particular amino acid or peptide, may be peptide ligands within the context of the invention. Such mimetics or analogs are characterized generally as exhibiting similar physical characteristics such as size, charge or hydrophobicity present in the appropriate spatial orientation as found in their peptide counterparts. A specific example of a peptide mimetic compound is a compound in which the amide bond between one or more of the amino acids is replaced by, for example, a carbon-carbon bond or other bond as is well known in the art (see, for example Sawyer, 1995, In: Peptide Based Drug Design pp. 378-422, ACS, Washington D.C.).

Therefore, the term “amino acid” within the scope of the present invention is used in its broadest sense and is meant to include naturally occurring L alpha-amino acids or residues. The commonly used one and three letter abbreviations for naturally occurring amino acids are used herein (Lehninger, A. L., 1975, Biochemistry, 2d ed., pp. 71-92, Worth Publishers, New York). The correspondence between the standard single letter codes and the standard three letter codes is well known to the skilled artisan, and is reproduced here: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; Y=Tyr. The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, that allow the same conformational restriction of the peptide compounds as natural Phe or Pro, are included within the definition of amino acid. Such analogs and mimetics are referred to herein as “functional equivalents” of an amino acid. Other examples of amino acids are listed by Roberts and Vellaccio, 1983, In: The Peptides: Analysis, Synthesis, Biology, Gross and Meiehofer, eds., Vol. 5 p. 341, Academic Press, Inc., N.Y., which is incorporated herein by reference.

Peptide ligands synthesized, for example, by standard solid phase synthesis techniques, are not limited to amino acids encoded by genes. Commonly encountered amino acids which are not encoded by the genetic code, include, for example, those described in International Publication No. WO 90/01940 such as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu and other aliphatic amino acids; 2-aminoisobutyric acid (Aib) for Gly; cyclohexylalanine (Cha) for Val, and Leu and Ile; homoarginine (Har) for Arg and Lys; 2,3-diaminopropionic acid (Dpr) for Lys, Arg and His; N-ethylglycine (EtGly) for Gly, Pro, and Ala; N-ethylglycine (EtGly) for Gly, Pro, and Ala; N-ethylasparigine (EtAsn) for Asn, and Gln; Hydroxyllysine (Hyl) for Lys; allohydroxyllysine (AHyl) for Lys; 3-(and 4)-hydoxyproline (3Hyp, 4Hyp) for Pro, Ser, and Thr; allo-isoleucine (AIle) for Ile, Leu, and Val; ρ-amidinophenylalanine for Ala; N-methylglycine (MeGly, sarcosine) for Gly, Pro, and Ala; N-methylisoleucine (MeIle) for Ile; Norvaline (Nva) for Met and other aliphatic amino acids; Norleucine (Nle) for Met and other aliphatic amino acids; Ornithine (Orn) for Lys, Arg and His; Citrulline (Cit) and methionine sulfoxide (MSO) for Thr, Asn and Gln; N-methylphenylalanine (MePhe), trimethylphenylalanine, halo (F, Cl, Br, and I) phenylalanine, trifluorylphenylalanine, for Phe.

Peptide ligands within the context of the present invention may be “engineered”, i.e., can be non-native or non-naturally occurring peptide ligands. By “non-native” or “non-naturally occurring” is meant that the amino acid sequence of the particular peptide ligand is not found in nature. That is to say, amino acid sequences of non-native or non-naturally occurring peptide ligands do not correspond to an amino acid sequence of a naturally occurring protein or polypeptide. Peptide ligands of this variety may be produced or selected using a variety of techniques well known to the skilled artisan. For example, constrained or unconstrained peptide libraries may be randomly generated and displayed on phage utilizing art standard techniques, for example, Lowman et al., 1998, Biochemistry 37:8870-8878.

Peptide ligands, when used within the context of the present invention, may be “conjugated” to a therapeutic or diagnostic substance. The term “conjugated” is used in its broadest sense to encompass all methods of attachment or joining that are known in the art. For example, in a typical embodiment, the therapeutic or diagnostic substance is a protein (referred to herein as a “protein therapeutic”), and the peptide ligand will be an amino acid extension of the C- or N-terminus of the protein therapeutic. In addition, a short amino acid linker sequence may lie between the protein therapeutic and the peptide ligand. In this scenario, the peptide ligand, optional linker and protein therapeutic will be encoded by a nucleic acid comprising a sequence encoding protein therapeutic operably linked (in the sense that the DNA sequences are contiguous and in reading frame) to an optional linker sequence encoding a short polypeptide as described below, and a sequence encoding the peptide ligand. In this typical scenario, the peptide ligand is considered to be “conjugated” to the protein therapeutic optionally via a linker sequence. In a related embodiment, the peptide ligand amino acid sequence may interrupt or replace a section of the protein therapeutic amino acid sequence, provided, of course, that the insertion of the peptide ligand amino acid sequence does not interfere with the function of the protein therapeutic. In this embodiment, the “conjugate” may be coded for by a nucleic acid comprising a sequence encoding protein therapeutic interrupted by and operably linked to a sequence encoding the peptide ligand. In a further typical embodiment, the peptide will be linked, e.g., by chemical conjugation to the protein therapeutic or other therapeutic optionally via a linker sequence. Typically, according to this embodiment, the peptide ligand will be linked to the protein therapeutic via a side chain of an amino acid somewhere in the middle of the protein therapeutic that doesn't interfere with the therapeutic's activity. Here again, the peptide is considered to be “conjugated” to the therapeutic.

As used within the context of the present invention the term “target molecule” includes, proteins, peptides, glycoproteins, glycopeptides, glycolipids, polysaccharides, oligosaccharides, nucleic acids, and the like. Target molecules include, for example, extracellular molecules such as various serum factors including but not limited to plasma proteins such as serum albumin, immunoglobulins, apolipoproteins, or transferrin, or proteins found on the surface of erythrocytes or lymphocytes, provided, of course, that binding of the peptide ligand to the cell surface protein does not substantially interfere with the normal function of the cell.

“Antibodies” and “immunoglobulins” are usually heterotetrameric glycoproteins of about 150,000 Daltons, composed of two identical light (L) chains and two identical heavy (H) chains.

Papain digestion of antibodies produces two identical antigen-binding fragments, called “Fab” fragments or regions, each with a single antigen-binding site, and a residual “Fc” fragment or region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof.

Pepsin treatment yields an F(ab′)₂ fragment that has two antigen-combining sites and is still capable of cross-linking antigen. The Fab′ fragment contains the constant domain of the light chain and the first constant domain (CH1) of the heavy chain.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the compositions comprising the peptide ligands of the invention. This includes chronic and acute disorders or diseases including those pathological conditions which predispose the mammal to the disorder in question.

“Elimination half-time” is used in its ordinary sense, as is described in Goodman and Gillman's The Pharmaceutical Basis of Therapeutics, pp. 21-25 Alfred Goodman Gilman, Louis S. Goodman, and Alfred Gilman, eds., 6th ed. 1980. Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2}, the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

“Transfection” refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

“Transformation” means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., 1989, Molecular Cloning (2nd ed.), Cold Spring Harbor Laboratory, NY, is generally used for prokaryotes or other cells that contain substantial cell-wall barriers. Infection with Agrobacterium tumefaciens is used for transformation of certain plant cells, as described by Shaw et al., 1983 Gene, 23:315 and WO 89/05859, published 29 Jun. 1989. For mammalian cells without such cell walls, the calcium phosphate precipitation method described in sections 16.30-16.37 of Sambrook et al., supra, is preferred. General aspects of mammalian cell host system transformations have been described by Axel in U.S. Pat. No. 4,399,216, issued 16 Aug. 1983. Transformations into yeast are typically carried out according to the method of Van Solingen et al., 1977, J. Bact., 130:946 and Hsiao et al., 1979, Proc. Natl. Acad. Sci. (USA), 76:3829. However, other methods for introducing DNA into cells such as by nuclear injection, electroporation, or by protoplast fusion may also be used.

As used herein, the term “pulmonary administration” refers to administration of a formulation of the invention through the lungs by inhalation. As used herein, the term “inhalation” refers to intake of air to the alveoli. In specific examples, intake can occur by self-administration of a formulation of the invention while inhaling, or by administration via a respirator, e.g., to an patient on a respirator. The term “inhalation” used with respect to a formulation of the invention is synonymous with “pulmonary administration.”

As used herein, the term “parenteral” refers to introduction of a compound of the invention into the body by other than the intestines, and in particular, intravenous (i.v.), intraarterial (i.a.), intraperitoneal (i.p.), intramuscular (i.m.), intraventricular, and subcutaneous (s.c.) routes.

As used herein, the term “aerosol” refers to suspension in the air. In particular, aerosol refers to the particlization of a formulation of the invention and its suspension in the air. According to the present invention, an aerosol formulation is a formulation comprising a compound of the present invention that is suitable for aerosolization, i.e., particlization and suspension in the air, for inhalation or pulmonary administration.

As used herein, the term “allometric scaling” refers to the extrapolation of animal data to assess pharmacokinetic parameters in humans, generally based on the power function Y=aWb, where the body weight (W) of the species is plotted against the pharmacokinetic parameter of interest on a log-log scale (see, for example, Mahmood, I. and Balian, J. D., Clin. Pharmacokinet 36(1):1-11 (1999)).
II. Modes for Carrying Out the Invention

A. Peptide Ligands

Peptide ligands within the context of the present invention bind a target, preferably a serum protein such as serum albumin or an immunoglobulin, and can be identified in a direct binding assay, or by their ability to compete for target binding with a known ligand for the target. Preferred peptide ligands that bind serum albumin include linear and cyclic peptides, preferably cyclic peptide compounds comprising the following formulae or are peptides that compete for binding serum albumin of a particular mammalian species with peptides of the following formulae:

(SEQ ID NO: 1)

Xaa-Xaa-Cys-Xaa-Xaa-Xaa-Xaa-Xaa-Cys-Xaa-Xaa
Phe-Cys-Xaa-Asp-Trp-Pro-Xaa-Xaa-Xaa-Ser-Cys

(SEQ ID NO: 2)

Val-Cys-Tyr-Xaa-Xaa-Xaa-Ile-Cys-Phe

(SEQ ID NO: 3)

Cys-Tyr-Xaa₁-Pro-Gly-Xaa-Cys
and

(SEQ ID NO: 4)

Asp-Xaa-Cys-Leu-Pro-Xaa-Trp-Gly-Cys-Leu-Trp

Preferred are peptide compounds of the foregoing general formulae comprising additional amino acids at the N-terminus (Xaa)ₓ and additional amino acids at the C-terminus (Xaa)_{z}, wherein Xaa is an amino acid and x and z are a whole number greater or equal to 0 (zero), generally less than 100, preferably less than 10 and more preferably 0, 1, 2, 3, 4 or 5 and more preferably 4 or 5 and wherein Xaa₁ is selected from the group consisting of Ile, Phe, Tyr and Val.

Further preferred peptide ligands that bind a serum albumin are identified as described herein in the context of the following general formulae: Trp-Cys-Asp-Xaa-Xaa-Leu-Xaa-Ala-Xaa-Asp-Leu-Cys (SEQ ID NO: 5) and Asp-Leu-Val-Xaa-Leu-Gly-Leu-Glu-Cys-Trp (SEQ ID NO: 6)

where additional amino acids may be present at the N-terminal end (Xaa)ₓ and additional amino acids may be present at the C-terminal end (Xaa)_{z}, and where Xaa is an amino acid and x and z are a whole number greater or equal to zero, generally less than 100, preferably less than 10 and more preferably 0, 1, 2, 3, 4 or 5 and more preferably 4 or 5.

According to this aspect of the invention reference is made to the Examples below and particularly the Tables contained showing and especially exemplary peptides and appropriate amino acids for selecting peptides ligands that bind a mammalian serum albumin. In a preferred aspect, reference is made to Table 7 for selecting peptide ligands that bind across several species of serum albumin.

Preferred compounds according to this aspect of the invention include:

| | |
|---|---|
| DLCLRDWGCLW | (SEQ ID NO:7) |
| DICLPRWGCLW | (SEQ ID NO:8) |
| MEDICLPRWGCLWGD | (SEQ ID NO:9) |
| QRLMEDICLPRWGCLWEDDE | (SEQ ID NO:10) |
| QGLIGDICLPRWGCLWGRSV | (SEQ ID NO:11) |
| QGLIGDICLPRWGCLWGRSVK | (SEQ ID NO:12) |
| EDICLPRWGCLWEDD | (SEQ ID NO:13) |
| RLMEDICLPRWGCLWEDD | (SEQ ID NO:14) |
| MEDICLPRWGCLWEDD | (SEQ ID NO:15) |
| MEDICLPRWGCLWED | (SEQ ID NO:16) |
| RLMEDICLARWGCLWEDD | (SEQ ID NO:17) |
| EVRSFCTRWPAEKSCKPLRG | (SEQ ID NO:18) |
| RAPESFVCYWETICFERSEQ | (SEQ ID NO:19) |
| EMCYFPGICWM | (SEQ ID NO:20) |

In a preferred embodiment, peptide ligands of the present invention bind human serum albumin and can be identified by their ability to compete for binding of human serum albumin in an in vitro assay with peptide ligands having the general formulae shown below, where additional amino acids may be present at the N-terminal end (Xaa)ₓ and at the C-terminal end (Xaa)_{z}:

| | |
|---|---|
| D X C L P X W G C L W | (SEQ ID NO:4) |
| F C X D W P X X X S C | (SEQ ID NO:1) |
| V C Y X X X I C F | (SEQ ID NO:2) |
| C Y X₁ P G X C X | (SEQ ID NO:3) |

where Xaa is an amino acid, x and z are preferably 4 or 5, and Xaa₁ is selected from the group consisting of Ile, Phe, Tyr, and Val.

In particular embodiments, the human serum albumin binding peptide ligands of the present invention will compete with any of the peptide ligands represented in SEQ ID NO: 7-20 described herein above and preferably will compete with SEQ ID NO: 10 for binding human serum albumin.

As will be appreciated from the foregoing, the term “compete” and “ability to compete” are relative terms. Thus the terms, when used to describe the peptide ligands of the present invention, refer to peptide ligands that produce a 50% inhibition of binding of, for example the peptide represented by SEQ ID NO: 10, when present at 50 μM, preferably when present at 1 μM, more preferably 100 nM, and preferably when present at 1 nM or less in a standard competition assay as described herein. However, peptide ligands having an affinity for a serum albumin of less than about 1 nM and preferably between about 1 pM and 1 nM are equally likely to be peptide ligands within the context of the present invention.

For in vitro assay systems to determine whether a peptide or other compound has the “ability” to compete with a peptide ligand for binding to serum albumin as noted herein, the skilled artisan can employ any of a number of standard competition assays. Competitive binding assays rely on the ability of a labeled standard to compete with the test sample analyte for binding with a limited amount of ligand. The amount of analyte in the test sample is inversely proportional to the amount of standard that becomes bound to the ligand.

Thus, the skilled artisan may determine whether a peptide or other compound has the ability to compete with a peptide ligand for binding to albumin employing procedures that include, but are not limited to, competitive assay systems using techniques such as radioimmunoassays (RIA), enzyme immunoassays (EIA), preferably the enzyme linked immunosorbent assay (ELISA), “sandwich” immunoassays, immunoradiometric assays, fluorescent immunoassays, and immunoelectrophoresis assays, to name but a few.

For these purposes, the selected peptide ligand will be labeled with a detectable moiety (the detectably labeled peptide ligand hereafter called the “tracer”) and used in a competition assay with a candidate compound for binding albumin. Numerous detectable labels are available that can be preferably grouped into the following categories:

(a) Radioisotopes, such as ³⁵S, ¹⁴C, ¹²⁵I, ³H, and ¹³¹I. The peptide compound can be labeled with the radioisotope using techniques described in Coligen et al., 1991, eds., Current Protocols in Immunology, Volumes 1 and 2, Wiley-Interscience, New York, N.Y., for example. Radioactivity can be measured using scintillation counting.

(b) Fluorescent labels such as rare earth chelates (europium chelates) or fluorescein and its derivatives, rhodamine and its derivatives, dansyl, lissamine, phycoerythrin, and Texas Red are available. The fluorescent labels can be conjugated to the peptide compounds using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme preferably catalyzes a chemical alteration of the chromogenic substrate that can be measured using various techniques. For example, the enzyme may catalyze a color change in a substrate, that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example:

(i) Horseradish peroxidase (HRP) with hydrogen peroxidase as a substrate, where the hydrogen peroxidase oxidizes a dye precursor (e.g. ABTS, orthophenylene diamine (OPD) or 3,3′,5,5′-tetramethyl benzidine hydrochloride (TMB));

(ii) alkaline phosphatase (AP) with para-nitrophenyl phosphate as chromogenic substrate; and

(iii) β-D-galactosidase (β-D-Gal) with a chromogenic substrate (e.g. p-nitrophenyl-β-D-galactosidase) or fluorogenic substrate 4-methylumbelliferyl-β-D-galactosidase.

According to a particular assay, the tracer is incubated with immobilized target in the presence of varying concentrations of unlabeled candidate compound. Increasing concentrations of successful candidate compound effectively compete with binding of the tracer to immobilized target. The concentration of unlabeled candidate compound at which 50% of the maximally-bound tracer is displaced is referred to as the “IC₅₀” and reflects the IgG binding affinity of the candidate compound. Therefore a candidate compound with an IC₅₀ of 1 mM displays a substantially weaker interaction with the target than a candidate compound with an IC₅₀ of 1 μM.

In some phage display ELISA assays, binding affinity of a mutated (“mut”) sequence was directly compared of a control (“con”) peptide using methods described in Cunningham et al., 1994, EMBO J. 13:2508, and characterized by the parameter EC₅₀. Assays were performed under conditions where EC₅₀(con)/EC₅₀(mut) will approximate K_{d}(con)/K_{d}(mut).

Accordingly, the invention provides compounds “having the ability to compete” for target molecules such as human serum albumin binding in an in vitro assay as described. Preferably the compound has an IC₅₀ for the target such as human serum albumin of less than 1 μM. Preferred among these compound are compounds having an IC₅₀ of less than about 100 nM, and preferably less than about 10 nM or less than about 1 nM. In further preferred embodiments according to this aspect of the invention the compounds display an IC₅₀ for the target molecule such as or human serum albumin of less than about 100 pM and more preferably less than about 10 pM.

A preferred in vitro assay for the determination of a candidate compound's ability to compete with a peptide ligand described herein is as follows and is described more fully in the Examples. In preferred embodiments the candidate compound is a peptide. The ability of a candidate compound to compete with a labeled peptide ligand tracer for binding to human serum albumin is monitored using an ELISA. Dilutions of a candidate compound in buffer are added to microtiter plates coated with human serum albumin (as described in the Example Sections) along with tracer for 1 hour. The microtiter plate is washed with wash buffer and the amount of tracer bound to human serum albumin measured.

B. Peptide Ligand Combinations

The peptide ligand is linked to a bioactive compound to form a hybrid molecule that comprises a peptide ligand domain and an active domain. The bioactive compounds of the invention include any compound useful as a therapeutic or diagnostic agent. Non-limiting examples of bioactive compounds include polypeptides such as enzymes, hormones, cytokines, antibodies, or antibody fragments, as well as organic compounds such as analgesics, antipyretics, antiinflammatory agents, antibiotics, antiviral agents, anti-fungal drugs, cardiovascular drugs, drugs that affect renal function and electrolyte metabolism, drugs that act on the central nervous system, chemotherapeutic drugs, etc. The peptide ligand domain is optionally joined to an active domain, via a flexible linker domain.

The hybrid molecules of the present invention are constructed by combining a peptide ligand domain with a suitable active domain. Depending on the type of linkage and its method of production, the peptide ligand domain may be joined via its N- or C-terminus to the N- or C-terminus of the active domain. For example, when preparing the hybrid molecules of the present invention via recombinant techniques, nucleic acid encoding a peptide ligand will be operably linked to nucleic acid encoding the active domain sequence, optionally via a linker domain. Typically the construct encodes a fusion protein wherein the C-terminus of the peptide ligand is joined to the N-terminus of the active domain. However, especially when synthetic techniques are employed, fusions where, for example, the N-terminus of the peptide ligand is joined to the N- or C-terminus of the active domain also are possible.

In some instances, the peptide ligand domain may be inserted within the active domain molecule rather than being joined to the active domain at its N-or C-terminus. This configuration may be used to practice the invention so long as the functions of the peptide ligand domain and the active domain are preserved. For example, a peptide ligand may be inserted into a non-binding light chain CDR of an immunoglobulin without interfering with the ability of the immunoglobulin to bind to its target. Regions of active domain molecules that can accommodate peptide ligand domain insertions may be identified empirically (i.e., by selecting an insertion site, randomly, and assaying the resulting conjugate for the function of the active domain), or by sequence comparisons amongst a family of related active domain molecules (e.g., for active domains that are proteins) to locate regions of low sequence homology. Low sequence homology regions are more likely to tolerate insertions of peptide ligands domains than are regions that are well-conserved. For active domain molecules whose three-dimensional structures are known (e.g. from X-ray crystallographic or NMR studies), the three-dimensional structure may provide guidance as to peptide ligand insertion sites. For example, loops or regions with high mobility (i.e., large temperature or “B” factors) are more likely to accommodate peptide ligand domain insertions than are highly ordered regions of the structure, or regions involved in ligand binding or catalysis.

C. Linker Domains

The peptide ligand domain is optionally linked to the active domain via a linker. The linker component of the hybrid molecule of the invention does not necessarily participate, but may contribute to the function of the hybrid molecule. Therefore, the linker domain is defined as any group of molecules that provides a spatial bridge between the active domain and the peptide ligand domain.

The linker domain can be of variable length and makeup, however, it is the length of the linker domain and not its structure that is important for creating the spatial bridge. The linker domain preferably allows for the peptide ligand domain of the hybrid molecule to bind, substantially free of steric and/or conformational restrictions to the target molecule. Therefore, the length of the linker domain is dependent upon the character of the two “functional” domains of the hybrid molecule, i.e., the peptide ligand domain and the active domain.

One skilled in the art will recognize that various combinations of atoms provide for variable length molecules based upon known distances between various bonds. See, for example, Morrison and Boyd, 1997, Organic Chemistry, 3rd Ed., Allyn and Bacon, Inc., Boston, Mass. The linker domain may be a polypeptide of variable length. The amino acid composition of the polypeptide determines the character and length of the linker. In a preferred embodiment, the linker molecule comprises a flexible, hydrophilic polypeptide chain. Exemplary linker domains comprise one or more Gly and/or Ser residues, such as those described in the Example sections below.

D. Recombinant Synthesis

The present invention encompasses a composition of matter comprising an isolated nucleic acid, preferably DNA, encoding a peptide ligand or a hybrid molecule comprising a peptide ligand domain and a polypeptide active domain as described herein. DNAs encoding the peptides of the invention can be prepared by a variety of methods known in the art. These methods include, but are not limited to, chemical synthesis by any of the methods described in Engels et al. 1989, Agnew. Chem. Int. Ed. Engl. 28:716-734 (the entire disclosure of which is incorporated herein by reference) such as the triester, phosphite, phosphoramidite, and H-phosphonate chemical synthesis methods. In one embodiment, codons preferred by the expression host cell are used in the design of the encoding DNA. Alternatively, DNA encoding the peptides can be altered to encode one or more variants by using recombinant DNA techniques, such as site specific mutagenesis (Kunkel et al., 1991, Methods Enzymol., 204:125-139; Carter et al. 1986, Nucl. Acids Res. 13:4331; Zoller et al. 1982, Nucl. Acids Res. 10:6487), cassette mutagenesis (Wells et al. 1985, Gene 34:315), restriction selection mutagenesis (Carter, 1991, In: Directed Mutagenesis: A Practical Approach, M. J. McPherson, ed., IRL Press, Oxford), and the like.

According to preferred aspects described above, the nucleic acid encodes a peptide ligand capable of binding a target molecule. Target molecules include, for example, extracellular molecules such as various serum factors, including but not limited to, plasma proteins such as serum albumin, immunoglobulins, apolipoproteins or transferrin, or proteins found on the surface of erythrocytes or lymphocytes, provided, of course, that binding of the peptide ligand to the cell surface protein does not substantially interfere with the normal function of the cell. Preferred for use in the present invention are peptide ligands that bind serum albumin with a desired affinity, for example, with high affinity, or with an affinity that facilitates useful tissue uptake and diffusion of a bioactive molecule that is fused to the peptide ligand.

According to another preferred aspect of the invention, the nucleic acid encodes a hybrid molecule comprising a peptide ligand domain sequence and an active domain. In this aspect of the invention, the active domain may comprise any polypeptide compound useful as a therapeutic or diagnostic agent, e.g., enzymes, hormones, cytokines, antibodies, or antibody fragments. The nucleic acid molecule according to this aspect of the present invention encodes a hybrid molecule and the nucleic acid encoding the peptide ligand domain sequence is operably linked to (in the sense that the DNA sequences are contiguous and in reading frame) the nucleic acid encoding the biologically active agent. Optionally these DNA sequences may be linked through a nucleic acid sequence encoding a linker domain amino acid sequence.

According to this aspect, the invention further comprises an expression control sequence operably linked to the DNA molecule encoding a peptide of the invention, an expression vector, such as a plasmid, comprising the DNA molecule, where the control sequence is recognized by a host cell transformed with the vector, and a host cell transformed with the vector. In general, plasmid vectors contain replication and control sequences derived from species compatible with the host cell. The vector ordinarily carries a replication site, as well as sequences that encode proteins capable of providing phenotypic selection in transformed cells.

For expression in prokaryotic hosts, suitable vectors include pBR322 (ATCC No. 37,017), phGH107 (ATCC No. 40,011), pBO475, pS0132, pRIT5, any vector in the pRIT20 or pRIT30 series (Nilsson and Abrahmsen 1990, Meth. Enzymol. 185:144-161), pRIT2T, pKK233-2, pDR540, and pPL-lambda. Prokaryotic host cells containing the expression vectors of the present invention include E. coli K12 strain 294 (ATCC NO. 31,446), E. coli strain JM101 (Messing et al. 1981, Nucl. Acid Res. 9:309), E. coli strain B, E. coli Strain_{—}1776 (ATCC No. 31537), E. coli c600, E. coli W3110 (F-, gamma-, prototrophic, ATCC No. 27,325), E. coli strain 27C7 (W3110, tonA, phoA E15, (argF-lac)169, ptr3, degP41, ompT, kan^{r}) (U.S. Pat. No. 5,288,931, ATCC No. 55,244), Bacillus subtilis, Salmonella typhimurium, Serratia marcesans, and Pseudomonas species.

In addition to prokaryotes, eukaryotic organisms, such as yeasts, or cells derived from multicellular organisms can be used as host cells. For expression in yeast host cells, such as common baker's yeast or Saccharomyces cerevisiae, suitable vectors include episomally-replicating vectors based on the 2-micron plasmid, integration vectors, and yeast artificial chromosome (YAC) vectors. For expression in insect host cells, such as Sf9 cells, suitable vectors include baculoviral vectors. For expression in plant host cells, particularly dicotyledonous plant hosts, such as tobacco, suitable expression vectors include vectors derived from the Ti plasmid of Agrobacterium tumefaciens.

Examples of useful mammalian host cells include monkey kidney CV1 line transformed by SV40 (COS-7, ATCC CRL 1651); human embryonic kidney line (293 or 293 cells subcloned for growth in suspension culture, Graham et al. 1977, J. Gen Virol. 36:59); baby hamster kidney cells (BHK, ATCC CCL 10); Chinese hamster ovary cells/-DHFR (CHO, Urlaub and Chasin 1980, Proc. Natl. Acad. Sci. USA, 77:4216); mouse sertoli cells (TM4, Mather 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al 1982, Annals N.Y. Acad. Sci. 383:44-68); MRC 5 cells; FS4 cells; and a human hepatoma cell line (Hep G2). For expression in mammalian host cells, useful vectors include vectors derived from SV40, vectors derived from cytomegalovirus such as the pRK vectors, including pRK5 and pRK7 (Suva et al. 1987, Science 237:893-896; EP 307,247 (Mar. 15, 1989), EP 278,776 (Aug. 17, 1988)) vectors derived from vaccinia viruses or other pox viruses, and retroviral vectors such as vectors derived from Moloney's murine leukemia virus (MoMLV).

Optionally, DNA encoding the peptide of interest is operably linked to a secretory leader sequence resulting in secretion of the expression product by the host cell into the culture medium. Examples of secretory leader sequences include STII, ecotin, lamB, herpes GD, lpp, alkaline phosphatase, invertase, and alpha factor. Also suitable for use herein is the 36 amino acid leader sequence of protein A (Abrahmsen et al. 1985, EMBO J. 4:3901).

Host cells are transfected and preferably transformed with the above-described expression or cloning vectors of this invention and cultured in conventional nutrient media modified as appropriate for inducing promoters, selecting transformants, or amplifying the genes encoding the desired sequences.

Prokaryotic host cells used to produce the present peptides can be cultured as described generally in Sambrook et al., supra.

The mammalian host cells used to produce peptides of the invention can be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in the art (for example, Ham and Wallace, 1979, Meth. Enz. 58:44; Barnes and Sato 1980, Anal. Biochem. 102:255, U.S. Pat. Nos. 4,767,704; 4,657,866; 4,927,762; or 4,560,655; WO 90/03430; WO 87/00195; U.S. Pat. Re. 30,985; or U.S. Pat. No. 5,122,469, the disclosure of each is incorporated herein by reference) may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in in vitro culture as well as cells that are within a host animal.

E. Chemical Synthesis

Another method of producing the compounds of the invention involves chemical synthesis. This can be accomplished by using methodologies well known in the art (see Kelley and Winkler, 1990, In: Genetic Engineering Principles and Methods, Setlow, J. K, ed., Plenum Press, N.Y., Vol. 12, pp 1-19; Stewart, et al., 1984, J. M. Young, J. D., Solid Phase Peptide Synthesis, Pierce Chemical Co., Rockford, Ill. See also U.S. Pat. Nos. 4,105,603; 3,972,859; 3,842,067; and 3,862,925).

Peptide ligands of the invention can be prepared conveniently using solid-phase peptide synthesis. Merrifield, 1964, J. Am. Chem. Soc. 85:2149; Houghten, 1985, Proc. Natl. Acad. Sci. USA 82:5132. Solid-phase peptide synthesis also can be used to prepare the hybrid molecule compositions of the invention if the active domain is or comprises a polypeptide.

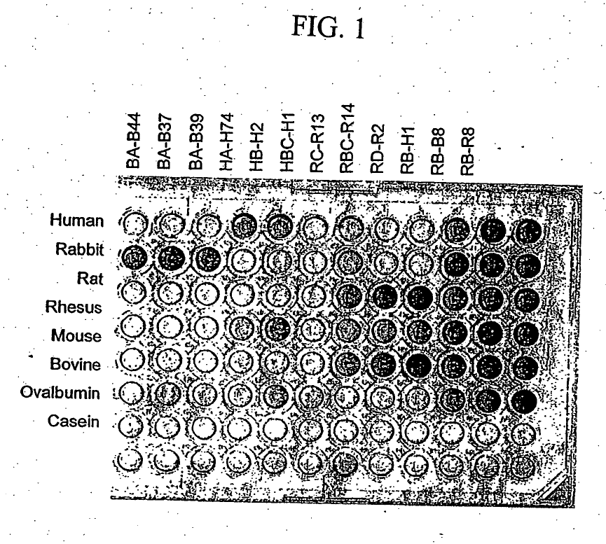

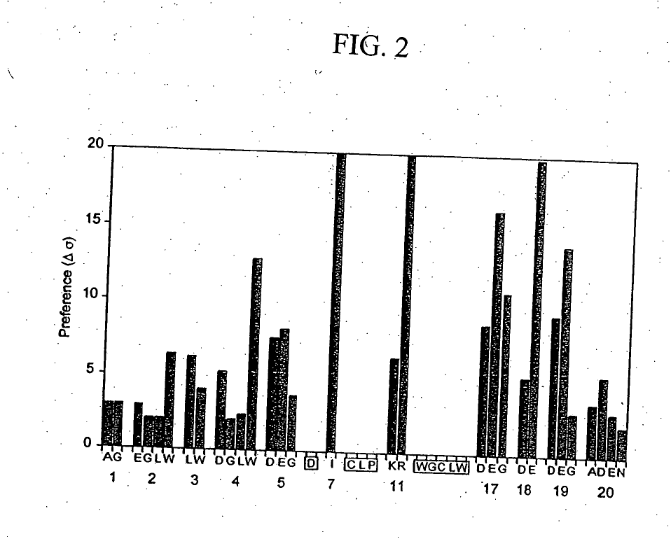

Solid-phase synthesis begins at the carboxy terminus of the nascent peptide by coupling a protected amino acid to an inert solid support. The inert solid support can be any macromolecule capable of serving as an anchor for the C-terminus of the initial amino acid. Typically, the macromolecular support is a cross-linked polymeric resin (e.g., a polyamide or polystyrene resin) as shown in FIGS. 1-1 and 1-2, on pages 2 and 4 of Stewart and Young, supra. In one embodiment, the C-terminal amino acid is coupled to a polystyrene resin to form a benzyl ester. A macromolecular support is selected such that the peptide anchor link is stable under the conditions used to deprotect the alpha-amino group of the blocked amino acids in peptide synthesis. If a base-labile alpha-protecting group is used, then it is desirable to use an acid-labile link between the peptide and the solid support. For example, an acid-labile ether resin is effective for base-labile Fmoc-amino acid peptide synthesis as described on page 16 of Stewart and Young, supra. Alternatively, a peptide anchor link and α-protecting group that are differentially labile to acidolysis can be used. For example, an aminomethyl resin such as the phenylacetamidomethyl (Pam) resin works well in conjunction with Boc-amino acid peptide synthesis as described on pages 11-12 of Stewart and Young, supra.

After the initial amino acid is coupled to an inert solid support, the alpha-amino protecting group of the initial amino acid is removed with, for example, trifluoroacetic acid (TFA) in methylene chloride and neutralized in, for example, triethylamine (TEA). Following deprotection of the initial amino acid's alpha-amino group, the next alpha-amino and side chain protected amino acid in the synthesis is added. The remaining alpha-amino and, if necessary, side chain protected amino acids are then coupled sequentially in the desired order by condensation to obtain an intermediate compound connected to the solid support. Alternatively, some amino acids may be coupled to one another to form a fragment of the desired peptide followed by addition of the peptide fragment to the growing solid phase peptide chain.

The condensation reaction between two amino acids, or an amino acid and a peptide, or a peptide and a peptide can be carried out according to the usual condensation methods such as the axide method, mixed acid anhydride method, DCC (N,N′-dicyclohexylcarbodiimide) or DIC (N,N′-diisopropylcarbodiimide) methods, active ester method, p-nitrophenyl ester method, BOP (benzotriazole-1-yl-oxy-tris [dimethylamino] phosphonium hexafluorophosphate) method, N-hydroxysuccinic acid imido ester method, etc., and Woodward reagent K method.

It is common in the chemical synthesis of peptides to protect any reactive side chain groups of the amino acids with suitable protecting groups. Ultimately, these protecting groups are removed after the desired polypeptide chain has been sequentially assembled. Also common is the protection of the alpha-amino group on an amino acid or peptide fragment while the C-terminal carboxy group of the amino acid or peptide fragment reacts with the free N-terminal amino group of the growing solid phase polypeptide chain, followed by the selective removal of the alpha-amino group to permit the addition of the next amino acid or peptide fragment to the solid phase polypeptide chain. Accordingly, it is common in polypeptide synthesis that an intermediate compound is produced that contains each of the amino acid residues located in the desired sequence in the peptide chain wherein individual residues still carry side-chain protecting groups. These protecting groups can be removed substantially at the same time to produce the desired polypeptide product following removal from the solid phase.

Alpha- and epsilon-amino side chains can be protected with benzyloxycarbonyl (abbreviated Z), isonicotinyloxycarbonyl (iNOC), o-chlorobenzyloxycarbonyl [Z(2C1)], p-nitrobenzyloxycarbonyl [Z(NO₂)], p-methoxybenzyloxycarbonyl [Z(OMe)], t-butoxycarbonyl (Boc), t-amyloxycarbonyl (Aoc), isobornyloxycarbonyl, adamantyloxycarbonyl, 2-(4-biphenyl)-2-propyloxycarbonyl (Bpoc), 9-fluorenylmethoxycarbonyl (Fmoc), methylsulfonyethoxycarbonyl (Msc), trifluoroacetyl, phthalyl, formyl, 2-nitrophenylsulphenyl (NPS), diphenylphosphinothioyl (Ppt), and dimethylphosphinothioyl (Mpt) groups, and the like.

Protective groups for the carboxy functional group are exemplified by benzyl ester (OBzl), cyclohexyl ester (Chx), 4-nitrobenzyl ester (ONb), t-butyl ester (Obut), 4-pyridylmethyl ester (OPic), and the like. It is often desirable that specific amino acids such as arginine, cysteine, and serine possessing a functional group other than amino and carboxyl groups are protected by a suitable protective group., For example, the guanidino group of arginine may be protected with nitro, p-toluenesulfonyl, benzyloxycarbonyl, adamantyloxycarbonyl, p-methoxybenzesulfonyl, 4-methoxy-2,6-dimethylbenzenesulfonyl (Nds), 1,3,5-trimethylphenysulfonyl (Mts), and the like. The thiol group of cysteine can be protected with p-methoxybenzyl, trityl, and the like.

Many of the blocked amino acids described above can be obtained from commercial sources such as Novabiochem (San Diego, Calif.), Bachem CA (Torrence, Calif.) or Peninsula Labs (Belmont, Calif.).

Stewart and Young, supra, provides detailed information regarding procedures for preparing peptides. Protection of alpha-amino groups is described on pages 14-18, and side chain blockage is described on pages 18-28. A table of protecting groups for amine, hydroxyl, and sulfhydryl functions is provided on pages 149-151.

After the desired amino acid sequence has been completed, the peptide can be cleaved away from the solid support, recovered, and purified. The peptide is removed from the solid support by a reagent capable of disrupting the peptide-solid phase link, and optionally deprotects blocked side chain functional groups on the peptide. In one embodiment, the peptide is cleaved away from the solid phase by acidolysis with liquid hydrofluoric acid (HF), which also removes any remaining side chain protective groups. Preferably, in order to avoid alkylation of residues in the peptide (for example, alkylation of methionine, cysteine, and tyrosine residues), the acidolysis reaction mixture contains thio-cresol and cresol scavengers. Following HF cleavage, the resin is washed with ether, and the free peptide is extracted from the solid phase with sequential washes of acetic acid solutions. The combined washes are lyophilized, and the peptide is purified.

F. Chemical Conjugation of Hybrids

In certain embodiments, the hybrid molecules may comprise active domains that are organic compounds having diagnostic or therapeutic utility, or alternatively, fusions between a peptide ligand domain and a polypeptide active domain in configurations that cannot be encoded in a single nucleic acid. Examples of the latter embodiment include fusions between the amino terminus of a peptide ligand and the amino terminus of the active domain, or fusions between the carboxy-terminus of a peptide ligand and the carboxy-terminus of the active domain.

Chemical conjugation may be employed to prepare these embodiments of the hybrid molecule, using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene, 2,6-diisocyanate), and bis-active fluorine compounds (such as 1,5-difluoro-2,4-dinitrobenzene).

G. Disulfide-Linked Peptides

As described above, some embodiments of the invention include cyclized peptide ligands. Peptide ligands may be cyclized by formation of a disulfide bond between cysteine residues. Such peptides can be made by chemical synthesis as described above and then cyclized by any convenient method used in the formation of disulfide linkages. For example, peptides can be recovered from solid phase synthesis with sulfhydryls in reduced form, dissolved in a dilute solution wherein the intramolecular cysteine concentration exceeds the intermolecular cysteine concentration in order to optimize intramolecular disulfide bond formation, such as a peptide concentration of 25 mM to 1 μM, and preferably 500 μM to 1 μM, and more preferably 25 μM to 1 μM, and then oxidized by exposing the free sulfhydryl groups to a mild oxidizing agent that is sufficient to generate intramolecular disulfide bonds, e.g., molecular oxygen with or without catalysts such as metal cations, potassium ferricyanide, sodium tetrathionate, and the like. Alternatively, the peptides can be cyclized as described in Pelton et al., 1986, J. Med. Chem. 29:2370-2375.

Cyclization can be achieved by the formation, for example, of a disulfide bond or a lactam bond between a first and a second residue capable of forming a disulfide bond, for example, Cys, Pen, Mpr, and Mpp and its 2-amino group-containing equivalents. Residues capable of forming a lactam bridge include, for example, Asp, Glu, Lys, Om, αβ-diaminobutyric acid, diaminoacetic acid, aminobenzoic acid, and mercaptobenzoic acid. The compounds herein can be cyclized for example via a lactam bond that can utilize the side chain group of a non-adjacent residue to form a covalent attachment to the N-terminus amino group of Cys or other amino acid. Alternative bridge structures also can be used to cyclize the compounds of the invention, including for example, peptides and peptidomimetics, that can cyclize via S—S, CH₂—S, CH₂—O—CH₂, lactam ester or other linkages.

H. Pharmaceutical Compositions

Pharmaceutical compositions which comprising the hybrid molecules of the invention may be administered in any suitable manner, including parental, topical, oral, or local (such as aerosol or transdermal), or any combination thereof.

Other suitable compositions of the present invention comprise any of the hybrid molecules noted above with a pharmaceutically acceptable carrier. The nature of the carrier differs with the mode of administration. For example, for oral administration, a solid carrier is preferred; for i.v. administration, a liquid salt solution carrier is generally used.

The compositions of the present invention include pharmaceutically acceptable components that are compatible with the subject and the protein of the invention. These generally include suspensions, solutions, and elixirs, and most especially biological buffers, such as phosphate buffered saline, saline, Dulbecco's Media, and the like. Aerosols may also be used, or carriers such as starches, sugars, microcrystalline cellulose, diluents, granulating agents, lubricants, binders, disintegrating agents, and the like (in the case of oral solid preparations, such as powders, capsules, and tablets).

As used herein, the term “pharmaceutically acceptable” generally means approved by a regulatory agency of the Federal or a state government or listed in the U.S. Pharmacopeia or other generally recognized pharmacopeia for use in animals, and more particularly in humans.

The formulation of choice can be accomplished using a variety of the aforementioned buffers, or even excipients including, for example, pharmaceutical grades of mannitol, lactose, starch, magnesium stearate, sodium saccharin cellulose, magnesium carbonate, and the like. “PEGylation” of the compositions may be achieved using techniques known to the art (see for example International Patent Publication No. WO92/16555, U.S. Pat. No. 5,122,614 to Enzon, and International Patent Publication No. WO92/00748).

A preferred route of administration of the present invention is in the aerosol or inhaled form. The compounds of the present invention, combined with a dispersing agent or dispersant, can be administered in an aerosol formulation as a dry powder or in a solution or suspension with a diluent.

As used herein, the term “dispersant” refers to an agent that assists aerosolization of the compound or absorption of the protein in lung tissue, or both. Preferably the dispersant is pharmaceutically acceptable. Suitable dispersing agents are well known in the art, and include but are not limited to surfactants and the like. For example, surfactants that are generally used in the art to reduce surface induced aggregation of a compound, especially a peptide compound, caused by atomization of the solution forming the liquid aerosol, may be used. Nonlimiting examples of such surfactants are surfactants such as polyoxyethylene fatty acid esters and alcohols, and polyoxyethylene sorbitan fatty acid esters. Amounts of surfactants used will vary, being generally within the range of from about 0.001% to about 4% by weight of the formulation. In a specific aspect, the surfactant is polyoxyethylene sorbitan monooleate or sorbitan trioleate. Suitable surfactants are well known in the art, and can be selected on the basis of desired properties, depending on the specific formulation, concentration of the compound, diluent (in a liquid formulation) or form of powder (in a dry powder formulation), and the like.

Moreover, depending on the choice of the peptide ligand, the desired therapeutic effect, the quality of the lung tissue (e.g., diseased or healthy lungs), and numerous other factors, the liquid or dry formulations can comprise additional components, as discussed further below.

The liquid aerosol formulations generally contain the peptide ligand/active domain hybrid and a dispersing agent in a physiologically acceptable diluent. The dry powder aerosol formulations of the present invention consist of a finely divided solid form of the peptide ligand/active domain hybrid and a dispersing agent. With either the liquid or dry powder aerosol formulation, the formulation must be aerosolized. That is, it must be broken down into liquid or solid particles in order to ensure that the aerosolized dose actually reaches the alveoli. In general the mass median dynamic diameter will be 5 micrometers or less in order to ensure that the drug particles reach the lung alveoli (Wearley, 1991, Crit. Rev. in Ther. Drug Carrier Systems 8:333). The term “aerosol particle” is used herein to describe the liquid or solid particle suitable for pulmonary administration, i.e., that will reach the alveoli. Other considerations such as construction of the delivery device, additional components in the formulation and particle characteristics are important. These aspects of pulmonary administration of a drug are well known in the art, and manipulation of formulations, aerosolization means and construction of a delivery device require at most routine experimentation by one of ordinary skill in the art.

With regard to construction of the delivery device, any form of aerosolization known in the art, including but not limited to nebulization, atomization or pump aerosolization of a liquid formulation, and aerosolization of a dry powder formulation, can be used in the practice of the invention. A delivery device that is uniquely designed for administration of solid formulations is envisioned. Often, the aerosolization of a liquid or a dry powder formulation will require a propellant. The propellant may be any propellant generally used in the art. Specific nonlimiting examples of such useful propellants are a chloroflourocarbon, a hydrofluorocarbon, a hydochlorofluorocarbon, or a hydrocarbon, including triflouromethane, dichlorodiflouromethane, dichlorotetrafuoroethanol, and 1,1,1,2-tetraflouroethane, or combinations thereof.

In a preferred aspect of the invention, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

Once the peptide ligand/active domain hybrid reaches the lung, a number of formulation-dependent factors affect the drug absorption. It will be appreciated that in treating a disease or disorder that requires circulatory levels of the compound, such factors as aerosol particle size, aerosol particle shape, the presence or absence of infection, lung disease or emboli may affect the absorption of the compounds. For each of the formulations described herein, certain lubricators, absorption enhancers, protein stabilizers or suspending agents may be appropriate. The choice of these additional agents will vary depending on the goal. It will be appreciated that in instances where local delivery of the compounds is desired or sought, such variables as absorption enhancement will be less critical.

I. Liquid Aerosol Formulations

The liquid aerosol formulations of the present invention will typically be used with a nebulizer. The nebulizer can be either compressed air driven or ultrasonic. Any nebulizer known in the art can be used in conjunction with the present invention such as but not limited to: Ultravent, Mallinckrodt, Inc. (St. Louis, Mo.); the Acorn II nebulizer (Marquest Medical Products, Englewood Co.). Other nebulizers useful in conjunction with the present invention are described in U.S. Pat. Nos. 4,624,251 issued Nov. 25, 1986; 3,703,173 issued Nov. 21, 1972; 3,561,444 issued Feb. 9, 1971 and 4,635,627 issued Jan. 13, 1971.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable elimination half-time. Such macromolecules include but are not limited to soya lecithin, oleic acid, and sorbetan trioleate, with sorbitan trioleate preferred.

The formulations of the present embodiment may also include other agents useful for protein stabilization or for the regulation of osmotic pressure. Examples of the agents include but are not limited to salts, such as sodium chloride, or potassium chloride, and carbohydrates, such as glucose, galactose, or mannose, and the like.

J. Aerosol Dry Powder Formulations

It is also contemplated that the present pharmaceutical formulation will be used as a dry powder inhaler formulation comprising a finely divided powder form of the peptide ligand and a dispersant. The form of the compound will generally be a lyophilized powder. Lyophilized forms of peptide ligand/active domain hybrid compounds can be obtained through standard techniques.

In another embodiment, the dry powder formulation will comprise a finely divided dry powder containing one or more compounds of the present invention, a dispersing agent and also a bulking agent. Bulking agents useful in conjunction with the present formulation include such agents as lactose, sorbitol, sucrose, or mannitol, in amounts that facilitate the dispersal of the powder from the device.

K. Research, Manufacturing, and Diagnostic Compositions

In one alternative embodiment, the peptide ligands can be utilized as purification reagents. For example, a gene encoding a peptide ligand is associated, in a vector, with a gene encoding a second protein, peptide, or fragment thereof. This results in the peptide ligand being produced by the host cell as a fusion with the second protein or peptide. The second protein or peptide is often a protein or peptide that can be secreted by the cell, making it possible to isolate and purify the second protein from the culture medium and eliminating the necessity of destroying the host cells which arises when the second protein remains inside the cell. Alternatively, the fusion protein can be expressed intracellularly. Highly expressed proteins that are preferred.

This use of the gene fusions is analogous to the use of Protein A fusions, where protein A, or more specifically the Z domain of protein A, binds to IgG and provides an “affinity handle” for the purification of the fused protein. Peptide ligands that bind serum albumin are similarly useful as “affinity handles” for the purification of fused proteins on a solid serum albumin support. For example, a DNA sequence encoding the desired peptide ligand can be fused by site directed mutagenesis to the gene for a protein or peptide. After expression and secretion, the fusion protein can be purified on a matrix of serum albumin to which the peptide ligand will bind. After purification, the peptide ligand can be enzymatically or chemically cleaved to yield free protein or left intact to aid in increasing the elimination half life of the fused protein. Fusion proteins can be cleaved using chemicals, such as cyanogen bromide, which cleaves at a methionine, or hydroxylamine, which cleaves between an Asn and Gly residue. Using standard recombinant DNA methodology, the nucleotide base pairs encoding these amino acids may be inserted just prior to the 5′ end of the gene encoding the desired peptide. Alternatively, one can employ proteolytic cleavage of fusion protein. See, for example, Carter, 1990, In: Protein Purification: From Molecular Mechanisms to Large-Scale Processes, Ladisch et al., eds., American Chemical Society Symposium Series No. 427, Ch. 13, pages 181-193.

The following examples are offered by way of illustration and not by way of limitation. The disclosures of all citations in the specification are expressly incorporated herein by reference.

### EXAMPLE 1

### Serum Albumin Peptide Ligands

Phage Libraries and Selection Conditions

Phage-displayed peptide libraries were selected against rabbit, rat, and human albumin. Phage libraries expressing random peptide sequences fused to the major coat protein, P8 (as described in Lowman et al., 1998 Biochem. 37, 8870) were pooled into 5 groups:

| | | |
|---|---|---|
| Pool A: | CX₂GPX₄C, | (SEQ ID NO: 21) |
| | X₄CX₂GPX₄CX₄, | (SEQ ID NO: 22) |
| and | | |
| | XᵢCXⱼCXₖ, | |
| | where j = 8-10; | |
| Pool B: | X₂₀ and | |
| | XᵢCXⱼCXₖ, | |
| | where j = 4-7; | |
| Pool C: | X₈ and | |
| | X₂CXⱼCX₂, | |
| | where j = 4-6; | |
| Pool D: | X₂CXⱼCX₂, | |
| | where j = 7-10; | |
| Pool E: | CX₆CX₆CCX₃CX₆C, | (SEQ ID NO: 23) |
| | CCX₃CX₆C, | (SEQ ID NO: 24) |
| | CCX₅CX₄CX₄CC, | (SEQ ID NO: 25) |
| and | | |
| | CXCX₇CX₃CX₆; | (SEQ ID NO: 26) |

where X represents any of the 20 naturally occurring L-amino acids. In each case (i+j+k)=18 and |i−k|<2. Each of the libraries contained excess of 10⁸ clones.

The phage library pools were suspended in binding buffer (PBS, 1% ovalbumin, 0.005% Tween 20) and sorted against rabbit, rat, or human albumin (Sigma, St. Louis Mo.) immobilized directly on Maxisorp plates (Nunc, Roskilde, Denmark) (10 μg/ml in PBS, overnight at 4° C.; plates were blocked for 1 hour at 25° C. with 1% ovalbumin in PBS, except for round 4, where TBS Blocker Casein (Pierce Chemical, Rockford, Ill.) was used. Phage were allowed to bind for with Blocker Casein for 2 hours. Unbound phage were removed by repetitive washing (PBS, 0.05% Tween 20) and bound phage were eluted with 500 mM KCl, 10 mM HCl, pH 2. Eluted phage were propagated in XL1-Blue cells with VCSM13 helper phage (Stratagene, La Jolla, Calif.). Enrichment was monitored by titering the number of phage that bound to an albumin coated well compared to a well coated with ovalbumin or casein.

Phage ELISA

Phage clones (approximately 10¹¹ phage) were added to Maxisorp plates coated with rat, rabbit, or human albumin or with mouse, bovine, or rhesus albumin (Sigma), as described above. The microtiter plate was washed with wash buffer and bound phage were detected following incubation with HRP/Anti-M13 Conjugate (Amerisham Pharmacia Biotech, Piscataway, N.J.). The amount of HRP bound was measured using ABTS/H₂O₂ substrate (Kirkegaard & Perry Laboratories, Gathersburg, Md.) and monitoring the change at 405 nm.

The peptide sequences displayed by phage clones selected for binding to rabbit, human, or rat albumin are shown below in Table 1. Also indicated is the ability of individual phage clones to bind the 3 species of immobilized albumin. This was tested using a phage ELISA. Note that clone RB, selected for binding to rat albumin is also capable of binding human and rabbit albumin.

TABLE 1

Species Specificity of Albumin-Binding Phage Peptides

Phage Binding

| | | | | |
|---|---|---|---|---|
| ID | Library | Rabbit | Human | Rat |

| | | | | | |
|---|---|---|---|---|---|
| | | Selected on Rabbit SA | | | |
| 27 | BA | G E N W C D S T L M A Y D L C G Q V N M | +++ | − | − |
| 28 | BB | M D E L A F Y C G I W E C L M H Q E Q K | +++ | − | − |
| 29 | BC | D L C D V D F C W F | +++ | − | − |
| 30 | BD | K S C S E L H W L L V E E C L F | +++ | − | − |
| | | Selected on Human SA | | | |
| 31 | HA | E V R S F C T D W P A E K S C K P L R G | − | +++ | − |
| 19 | HB | R A P E S F V C Y W E T I C F E R S E Q | − | ++ | (+) |
| 20 | HC | E M C Y F P G I C W M | − | +++ | ++ |
| 32 | HE | C E V A L D A C R G G E S G C C R H I C E L I R Q L C | − | (+) | − |
| | | Selected on Rat SA | | | |
| 33 | RA | R N E D P C V V L L E M G L E C W E G V | − | − | +++ |
| 34 | RD | D T C V D L V R L G L E C W G | − | − | +++ |
| 35 | RB | Q R Q M V D F C L P Q W G C L W G D G F | ++ | + | +++ |
| 7 | RC | D L C L R D W G C L W | − | − | +++ |
| 36 | RE | C G C V D V S D W D C W S E C L W S H G A | − | − | +++ |

Sequence Maturation on Monovalent Phage

Partially randomized libraries were designed using oligonucleotides coding for each of the selected clones in Table 1, but synthesized with a 70-10-10-10 mixture of bases as described in Dennis et al., 2000 Nature 404; 465. Although the potential diversity of these libraries is the same as the initial naive libraries, each ‘soft randomized’ library maintains a bias towards the selected sequence in Table 1. Each library was again selected for binding to rat, rabbit, or human albumin regardless of its origin. For example, the library resulting from soft randomization of clone RB was selected against rat, rabbit, or human albumin even though it was originally identified for binding to rat albumin. Sequences identified following soft randomization are shown in Table 2 along with their species specificity as determined by phage ELISA. Most clones appear to be specific for the species of albumin for which they were selected, however, clones from the RB soft randomization library bind to all three species.

TABLE 2

Binds

| | | | |
|---|---|---|---|
| ID | Human | Rabbit | Rat |

| | | | | | |
|---|---|---|---|---|---|
| | Sequences Selected on Rabbit Albumin | | | | |
| Library BA | G E N W C D S T L M A Y D L C G Q V N M | 27 | | | |
| BA-B44 | G E D W C D S T L L A F D L C G E G A R | 37 | − | +++ | − |
| BA-B37 | G E N W C D W V L L A Y D L C G E D N T | 38 | − | +++ | − |
| BA-B39 | M E L W C D S T L M A Y D L C G D F N M | 39 | − | +++ | − |
| | Sequences Selected on Human Albumin | | | | |
| Library HA | E V R S F C T D W P A E K S C K P L R G | 31 | | | |
| HA-H74 | E V R S F C T D W P A H Y S C T S L Q G | 40 | +++ | − | − |
| HA-H83 | G — R S F C M D W P A H K S C T P L M L | 41 | +++ | − | − |
| HA-H73 | G V R T F C Q D W P A H N S C K L L R G | 42 | +++ | − | − |
| HA-H76 | Q T R S F C A D W P R H E S C K P L R G | 43 | +++ | − | − |
| HA-H84 | R — R T — C — D W P — H N S C K — L R G | 44 | +++ | − | − |
| Library HB | R A P E S F V C Y W E T I C F E R S E Q | 19 | | | |
| HB-H2 | R A A E S S V C Y W P G I C F D R T E Q | 45 | +++ | − | − |
| HB-H8 | M E P S R S V C Y A E G I C F D R G E Q | 46 | +++ | − | − |
| HB-H3 | R E P A S L V C Y F E D I C F V R A E A | 47 | + | − | − |
| HB-H6 | R G P D — V — C Y W P S I C F E R S M P | 48 | + | − | − |
| HB-H4 | L V P E R I V C Y F E S I C Y E R S E L | 49 | + | − | − |
| HB-H16 | R M P A S L P C Y W E T I C Y E S S E Q | 50 | + | − | − |
| HB-H18 | R T A E S L V C Y W P G I C F A Q S E R | 51 | + | − | − |
| HB-H1 | R A P E R W V C Y W E G I C F D R Y E Q | 52 | (+) | − | − |
| Library HC | E M C Y F P G I C W M | 20 | | | |
| HB-H12 | E I C Y F P G I C W I | 53 | ++ | − | − |
| HB-H13 | E L C Y F P G I C W T | 54 | ++ | − | − |
| HC-H6 | D I C Y I P G I C W M | 55 | ++ | − | − |
| HC-H2 | K L C Y F P G I C W S | 56 | ++ | − | − |
| HC-H3 | D L C Y F P G I C W M | 57 | ++ | − | − |
| HC-H4 | G M C Y F P G I C W A | 58 | ++ | − | − |
| HC-H7 | E M C Y F P G I C W S | 59 | ++ | − | − |
| HC-H9 | E M C Y F P G I C W T | 60 | ++ | − | − |
| HC-H10 | K T C Y F P G I C W M | 61 | ++ | − | − |
| HC-H5 | K V C Y F P G I C W M | 62 | | | |
| HC-H8 | D V C V F P G I C W M | 63 | ++ | − | − |
| HC-H17 | E I C Y F P G I C W M | 64 | ++ | − | − |
| HC-H14 | A L C Y F P G I C W M | 65 | ++ | − | − |
| HG-H15 | E L C Y F P G I C W P | 66 | ++ | − | − |
| HC-H20 | E L C Y F P G I C W M | 67 | ++ | − | − |
| HC-H13 | D M C Y F P G I C W L | 68 | ++ | − | − |
| HC-H18 | D M C Y F P G I C F N | 69 | ++ | − | − |
| HC-H12 | E T C Y F P G I C W L | 70 | ++ | − | − |
| HC-H11 | E V C Y F P G I C W F | 71 | ++ | − | − |
| HC-H16 | E V C Y F P G I C W E | 72 | ++ | − | − |
| HC-H19 | E V C Y F P G I C W M | 73 | ++ | − | − |
| Library HBC | X X E M C Y F P G I C W M X X | 426 | | | |
| HBC-H7 | L A E M C Y F P G I C W M S A | 74 | +++ | − | − |
| HBC-H4 | G G E I C Y F P G I C R V L P | 75 | +++ | − | − |
| HBC-H6 | E H D M C Y F P G I C W I A D | 76 | +++ | − | − |
| HBC-H10 | V Q E V C Y F P G I C W M Q E | 77 | +++ | − | − |
| HBC-H2 | S R E V C Y Y P G I C W N G A | 78 | +++ | − | − |
| HBC-H1 | D S E V C V F P G I C W S G T | 79 | +++ | − | − |
| HBC-H3 | G T E V C Y F P G I C W G G G | 80 | +++ | − | − |
| HBC-H8 | S Y A P C Y F P G I C W M G N | 81 | +++ | − | − |
| HBC-H17 | H A E I C Y F P G I C W T E R | 82 | +++ | − | − |
| HBC-H11 | N D E I C Y F P G V C W K S G | 83 | +++ | − | − |
| HBC-H18 | R D T V C Y F P G I C W M A S | 84 | +++ | − | − |
| HBC-H19 | V R D M C Y F P G I C W K S E | 85 | +++ | − | − |
| HBC-H12 | A S E I C Y F P G I C W M V E | 86 | +++ | − | − |
| HBC-H13 | Q T E L C Y F P G I C W N E S | 87 | +++ | − | − |
| HBC-H14 | T T E M C Y F P G I C W K T E | 88 | +++ | − | − |
| HBC-H15 | K T E I C Y F P G I C W M S G | 89 | +++ | − | − |
| HBC-H16 | Q — — — C — F P G — — W V — K | 90 | +++ | − | − |
| HB-H10 | I V E M C Y Y P G I C W I S P | 91 | +++ | − | − |
| HB-H7 | S G A I C Y V P G I C W T H A | 92 | +++ | − | − |
| | Sequences Selected on Rat Albumin | | | | |
| Library RB | Q R Q M V D F C L P Q W G C L W G D G F | 35 | | | |
| RB-H1 | Q R H P E D I C L P R W G C L W G D D D | 93 | ++ | +++ | +++ |
| RB-H6 | N R Q M E D I C L P Q W G C L W G D D F | 94 | ++ | +++ | +++ |
| RB-B2 | Q R L M E D I C L P R W G C L W G D R F | 95 | ++ | +++ | +++ |
| RB-B5 | Q W H M E D I C L P Q W G C L W G D V L | 96 | ++ | +++ | +++ |
| RB-B6 | Q W Q M E N V C L P K W G C L W E E L D | 97 | ++ | +++ | +++ |
| RB-B4 | L W A M E D I C L P K W G C L W E D D F | 98 | ++ | +++ | +++ |
| RB-B7 | L R L M D N I C L P R W G C L W D D G F | 99 | ++ | +++ | +++ |
| RB-B8 | H S Q M E D I C L P R W G C L W G D E L | 100 | ++ | +++ | +++ |
| RB-B11 | Q W Q V M D I C L P R W G C L W A D E Y | 101 | ++ | +++ | +++ |
| RB-B12 | Q G L I G D I C L P R W G C L W G D S V | 11 | ++ | +++ | +++ |
| RB-B16 | H R L V E D I C L P R W G C L W G N D F | 102 | ++ | +++ | +++ |
| RB-B9 | Q M H M M D I C L P K W G C L W G D T S | 103 | (+) | +++ | +++ |
| RB-B14 | L R I F E D I C L P K W G C L W G E G F | 104 | (+) | +++ | +++ |
| RB-B3 | Q S Y M E D I C L P R W G C L S D D A S | 105 | (+) | +++ | +++ |
| RB-B10 | Q G D F W D I C L P R W G C L S G E G Y | 106 | − | +++ | +++ |
| RB-B1 | R W Q T E D V C L P K W G C L F G D G V | 107 | − | +++ | +++ |
| RB-R8 | Q G L I G D I C L P R W G C L W G D S V | 11 | ++ | +++ | +++ |
| RB-R16 | L I F M E D V C L P O W G C L W E D G V | 108 | ++ | +++ | +++ |
| HC-R10 | Q R D M G D I C L P R W G C L W E D G V | 109 | ++ | +++ | +++ |
| RB-R4 | Q R H M M D F C L P K W G C L W G D G Y | 110 | − | (+) | +++ |
| RB-R7 | Q R P I M D F C L P K W G C L W E D G F | 111 | − | (+) | +++ |
| RB-R11 | E R Q M V D F C L P K W G C L W G D G F | 112 | − | (+) | +++ |
| RB-R12 | Q G Y M V D F C L P R W G C L W G D A N | 113 | − | (+) | +++ |
| RB-R13 | K M G R V D F C L P K W G C L W G D E L | 114 | − | (+) | +++ |
| RB-R15 | Q S Q L E D F C L P K W G C L W G D G F | 115 | − | (+) | +++ |
| RB-R17 | Q G G M G D F C L P Q W G C L W G E D L | 116 | − | (+) | +++ |
| RB-R5 | Q R L M W E I C L P L W G C L W G D G L | 117 | − | − | +++ |
| RB-R10 | Q R Q I M D F C L P H W G C L W G D G F | 118 | − | − | +++ |
| RB-R2 | G R Q V V D F C L P K W G C L W E E G L | 119 | − | − | +++ |
| RB-R3 | Q M Q M S D F C L P Q W G C L W G D G Y | 120 | − | − | +++ |
| RB-R9 | K S R M G D F C L P E W G C L W G D E L | 121 | − | − | +++ |
| RB-R1 | E R Q M E D F C L P Q W G C L W G D G V | 122 | − | − | +++ |
| RB-R14 | Q R Q V V D F C L P Q W G C L W G D G S | 123 | − | − | +++ |
| Library RC | D L C L R D W G C L W | 7 | | | |
| RC-R6 | D I C L P E W G C L W | 124 | − | − | ++ |
| RC-R8 | D I C L P E W G C L W | 124 | − | − | ++ |
| RO-R15 | D I C L P E W G C L W | 124 | − | − | ++ |
| RO-R1 | D I C L P V W G C L W | 125 | − | − | ++ |
| RC-R2 | D I C L P V W G C L W | 125 | − | − | ++ |
| RC-R3 | D I C L P V W G C L W | 125 | − | − | ++ |
| RC-R10 | D I C L P V W G C L W | 125 | − | − | ++ |
| RC-R12 | D I C L P V W G C L W | 125 | − | − | ++ |
| RC-R18 | D I C L P V W G C L W | 125 | − | − | ++ |
| RC-R9 | D L C L P E W G C L W | 126 | − | − | (+) |
| RC-R4 | D L C L P K W G C L W | 127 | − | − | ++ |
| RC-R5 | D L C L P V W G C L W | 128 | − | − | (+) |
| RC-R20 | D I C L P A W G C L W | 129 | − | − | ++ |
| RC-R17 | D I C L P D W G C L W | 130 | − | − | ++ |
| RC-R13 | D I C L P R W G C L W | 8 | − | − | ++ |
| RC-R16 | D I C L E R W G C L W | 131 | − | − | ++ |
| Library RB | C X X D L C L R D W G C L W X X | 427 | | | |
| RBC-R16 | E W D V C L P H W G C L W D G | 132 | − | (+) | +++ |
| RBC-R7 | W D D I C F R D W G C L W G S | 133 | − | − | +++ |
| RBC-R1 | M D D I C L H H W G C L W D E | 134 | − | − | +++ |
| RBC-R2 | M D D L C L P N W G C L W G D | 135 | − | − | +++ |
| RBC-R4 | F E D F C L P N W G C L W G S | 136 | − | − | +++ |
| RBC-R6 | F E D L C V V R W G C L W G D | 137 | − | − | +++ |
| RBC-R5 | W E D L C L P D W G C L W E D | 138 | − | − | +++ |
| RBC-R9 | S E D F C L P V W G C L W E D | 139 | − | − | +++ |
| RBC-R10 | D F D L C L P D W G C L W D D | 140 | − | − | +++ |
| RBC-R8 | N W D L C F P D W G C L W D D | 141 | − | − | +++ |
| RBC-R14 | E E D L C L P V W G C L W G A | 142 | − | − | +++ |
| RBC-R20 | E E D V C L P V W G C L W E G | 143 | − | − | +++ |
| RBC-R12 | M F D L C L P K W G C L W G N | 144 | − | − | +++ |
| RBC-R13 | E F D L C L P T W G C L W E D | 145 | − | − | +++ |
| RBC-R15 | M W D V C F P D W G C L W D V | 146 | − | − | +++ |
| RBC-R18 | E W D V C F P A W G C L W D Q | 147 | − | − | +++ |
| RBC-R11 | V W D L C L P O W G C L W D E | 148 | − | − | +++ |
| Library RD | D T C V D L V R L G L E C W G | 34 | | | |
| RD-R2 | D T C A D L V R L G L E C W A | 149 | − | − | +++ |
| RD-R7 | N T C A D L V R L G L E C W A | 150 | − | − | +++ |
| RD-R11 | D T C D D L V Q L G L E C W A | 151 | − | − | +++ |
| RD-R5 | D T C E D L V R L G L E C W A | 152 | − | − | +++ |
| RD-R6 | D S C G D L L R L G L E C W A | 153 | − | − | +++ |
| RD-R1 | D T C S D L V G L G L E C W A | 154 | − | − | +++ |

Phage clones were also tested for binding to rhesus, mouse, and bovine albumin. Clones originating from the RB soft randomization library were found to bind each of these species of albumin. Binding to albumin was specific, as demonstrated by a lack of binding to ovalbumin and casein (FIG. 1). Some clones that bind to multiple species of albumin (multi-species binders) are listed in Table 3.

TABLE 3

Multi Species Binders

Binds

| | | | | |
|---|---|---|---|---|
| Phage | ID | Human | Rabbit | Rat |

| | | | | | |
|---|---|---|---|---|---|
| RB | Q R Q M V D F C L P Q W G C L W G D G F | 35 | + | ++ | +++ |
| RB-H1 | Q R H P E D I C L P R W G C L W G D D D | 93 | ++ | +++ | +++ |
| RB-H6 | N R Q M E D I C L P Q W G C L W G D D F | 94 | ++ | +++ | +++ |
| RB-B12 | Q G L I G D I C L P R W G C L W G D S V | 11 | ++ | +++ | +++ |
| RB-B8 | H S Q M E D I C L P R W G C L W G D E L | 100 | ++ | +++ | +++ |
| RB-B7 | L R L M D N I C L P R W G C L W D D G F | 99 | ++ | +++ | +++ |
| RB-B5 | Q W H M E D I C L P Q W G C L W G D V L | 96 | ++ | +++ | +++ |
| RB-B6 | Q W Q M E N V C L P K W G C L W E E L D | 97 | ++ | +++ | +++ |
| RB-B4 | L W A M E D I C L P K W G C L W E D D F | 98 | ++ | +++ | +++ |
| RB-B11 | Q W Q V M D I C L P R W G C L W A D E V | 101 | ++ | +++ | +++ |
| RB-B16 | H R L V E D I C L P R W G C L W G N D F | 102 | ++ | +++ | +++ |
| RB-B2 | Q R L M E D I C I P R W G C L W G D R F | 95 | ++ | +++ | +++ |
| RB-R8 | Q G L I G D I C L P R W G C L W G D S V | 11 | ++ | +++ | +++ |
| RB-R16 | L I F M E D V C L P Q W G C L W E D G V | 108 | ++ | +++ | +++ |
| HC-R10 | Q R D M G D I C L P R W G C L W E D G V | 109 | ++ | +++ | +++ |

Hard Randomization

Sequences from soft randomization of the RB sequence were further matured using a hard randomization strategy. A new, fully randomized library was designed around a core sequence of highly selected residues: DXCLPXWGCLW (SEQ ID NO: 423) that kept highly selected residues constant: X₅DXCLPXWGCLWX₄ (SEQ ID NO: 155), while fully randomizing the remaining positions. A second library, one residue shorter at both the N and C terminus was also constructed, X₄DXCLPXWGCLWX₃ (SEQ ID NO: 156). The sequence preferences at each randomized position resulting from selection against rabbit albumin are shown in FIG. 2. Similar profiles were observed from sequences selected from binding rat and human albumin (data not shown). For each species of albumin, there was a strong preference for Ile at position 7 and Arg at position 11, generating the core consensus peptide: DICLPRWGCLW (SEQ ID NO: 8). Additionally, this was a general preference for negatively charged residues (Asp or Glu at position flanking this core, particularly on the carboxy terminus. Sequences obtained from these libraries, selected against rat, rabbit, and human albumin, are shown in Tables 4, 5, and 6, respectively.

TABLE 4

Sequences Selected on Rat Albumin

| | |
|---|---|
| clone ID | Hard Randomization Library |

| | | |
|---|---|---|
| | 155 | X X X X X D X C L P X W G C L W X X X X |
| 35 | 157 | A A Q V G D I C L P R W G C L W S E Y A |
| 33 | 8 | A G W A A D V C L P R W G C L W E E D V |
| 60 | 9 | A S V V D D I C L P V W G C L W G E D I |
| 84 | 160 | A T M E D D I C L P R W G C L W G A E E |
| 10 | 161 | D E D F E D Y C L P P W G C L W G S S M |
| 34 | 162 | E G T W D D F C L P R W G C L W L G E R |
| 93 | 163 | E R W E G D V C L P R W G C L W G E S G |
| 23 | 164 | G D W M H D I C L P K W G C L W D E K A |
| 71 | 165 | G I E W G D T C L P K W G C L W R V E G |
| 36 | 166 | G Q Q G E D V C L P V W G C L W D T S S |
| 48 | 167 | G R Y P M D L C L P R W G C L W E D S A |
| 24 | 168 | G S A G D D L C L P R W G C L W E R G A |
| 9 | 169 | H A S D W D V C L P G W G C L W E E D D |
| 47 | 170 | L G V T H D T C L P R W G C L W D E V G |
| 72 | 171 | L V W E E D F C L P K W G C L W G A E D |
| 11 | 172 | N V G W N D I C L P R W G C L W A Q E S |
| 83 | 173 | Q G V E W D V C L P Q W G C L W T R E V |
| 58 | 174 | R L D A W D I C L P Q W G C L W E E P S |
| 96 | 175 | S E A P G D Y C L P R W G C L W A Q E K |
| 94 | 176 | T A M D E D V C L P R W G C L W G S G S |
| 81 | 177 | T E I G Q D F C L P R W G C L W V P G T |
| 57 | 178 | T L G W P D F C L P K W G C L W R E S D |
| 12 | 179 | T L S N Q D I C L P G W G C L W G G I N |
| 46 | 180 | T S T G G D L C L P R W G C L W D S S E |
| 22 | 181 | V S E M D D I C L P L W G C L W A D A P |
| 59 | 182 | V S E W E D I C L P S W G C L W E T Q D |
| 45 | 183 | V V G D G D F C L P K W G C L W D Q A R |
| 21 | 184 | V V W D D D V C L P R W G C L W E E Y G |
| 69 | 185 | W S D S D D V C L P R W G C L W G N V A |
| 95 | 186 | W V E E G D I C L P R W G C L W E S V E |
| 33 | 187 | A Q A M G D I C L P R W G C L W E A E I |
| 10 | 188 | A S D R G D L C L P Y W G C L W G P D G |
| | 155 | X X X X X D X C L P X W G C L W X X X X |
| 93 | 189 | X X X X X D X C L P X W G C L W X X X X |
| 71 | 190 | A S D P G D V C L P R W G C L W G E S F |
| 22 | 191 | A S T P R D I C L P R W G C L W S E D A |
| 23 | 192 | D G E E G D L C L P R W G C L W A L E H |
| 24 | 193 | E G E E V D I C L P Q W G C L W G Y P V |
| 82 | 194 | E V G D L D L C L P R W G C L W G N D K |
| 81 | 195 | F R D G E D F C L P Q W G C L W A D T S |
| 46 | 196 | G D M V N D F C L P R W G C L W G S E N |
| 83 | 197 | G R M G T D L C L P R W G C L W G E V E |
| 94 | 198 | H E W E R D I C L P R W G C L W R D G E |
| 35 | 199 | K K V S G D I C L P I W G C L W D N D Y |
| 96 | 200 | L L E S D D I C L P R W G C L W H E D G |
| 21 | 201 | M Q A E S D F C L P H W G C L W D E G T |
| 36 | 202 | M Q G P L D I C L P R W G C L W G G V D |
| 48 | 203 | Q M P L E D I C L P R W G C L W E G R E |
| 95 | 204 | R E E W G D L C L P T W G C L W E T K K |
| 47 | 205 | R V W T E M V C L P R W G C L W S E G N |
| 11 | 206 | S I R E Y D V C L P K W G C L W E P S A |
| 34 | 207 | S P T E W D M C L P K W G C L W G D A L |
| 69 | 208 | S S G L E D I C L P N W G C L W A D G S |
| 9 | 209 | S V G W G D I C L P V W G C L W G E G G |
| 57 | 210 | T E E N W D L C L P R W G C L W G D D W |
| 84 | 211 | T S G S D D I C L P V W G C L W G E D S |
| 58 | 212 | T W P — G D L C L P R W G C L W E A E S |
| 72 | 213 | W D H E L D F C L P V W G C L W A E D V |
| 60 | 214 | W T E S E D I C L P G W G C L W G P E V |
| 59 | 215 | W V P F E D V C L P R W G C L W S S Y Q |
| | 156 | X X X X D X C L P X W G C L W X X X |
| 93 | 216 | E E D S D I C L P R W G C L W N T S |
| 81 | 217 | E G Y W D L C L P R W G C L W E L E |
| 10 | 218 | E L G E D L C L P R W G C L W G S E |
| 24 | 219 | E T W S D V C L P R W G C L W G A S |
| 83 | 220 | G D Y V D L C L P G W G C L W E D G |
| 23 | 221 | G V L D D I C L P R W G C L W G P K |
| 94 | 222 | H M M D D V C L P G W G C L W A S E |
| 59 | 223 | I D Y T D L C L P A W G C L W E L E |
| 36 | 224 | I E H E D L C L P R W G C L W A V D |
| 11 | 225 | I S E W D L C L P R W G C L W D R S |
| 12 | 226 | I S W A D V C L P K W G C L W G K D |
| 47 | 227 | I S W G D L C L P R W G C L W E G S |
| 22 | 228 | K L W D D I C L P R W G C L W S P L |
| 84 | 229 | L A W P D V C L P R W G C L W G G M |
| 71 | 230 | L N E S D I C L P T W G C L W G V D |
| 46 | 231 | L P E Q D V C L P V W G C L W D A N |
| 35 | 232 | M A W G D V C L P R W G C L W A G G |
| 48 | 233 | N E E W D V C L P R W G C L W G G V |
| 60 | 234 | Q E L Q D F C L P R W G C L W G V G |
| 21 | 235 | Q R E W D V C L P R W G C L W S D V |
| 9 | 236 | Q R F W D T C L P R W G C L W G G D |
| 57 | 237 | R V F T D V C L P R W G C L W D L G |
| 58 | 238 | S G W D D V C L P V W G C L W G P S |
| 96 | 239 | S S A S D Y C L P R W G C L W G D L |
| 72 | 240 | S W Q G D I C L P R W G C L W G V D |
| 69 | 241 | S Y E T D V C L P Y W G C L W E D A |
| 34 | 242 | S Y W G D V C L P R W G C L W S E A |
| 45 | 243 | T L E W D M C L P R W G C L W T E Q |
| 95 | 244 | V G E F D I C L P R W G C L W D A E |
| 33 | 245 | V T S W D V C L P R W G C L W E E D |
| 82 | 246 | W L W E D L C L P K W G C L W E E D |
| 82 | 247 | A L F E D V C L P V W G C L W G G E |
| 45 | 248 | A S E W D V C L P T W G C L W M E G |
| 34 | 249 | A Y S A D I C L P R W G C L W M S E |
| | 156 | X X X X D X C L P X W G C L W X X X |
| 35 | 250 | E D W E D I C L P Q W G C L W E G M |
| 83 | 251 | E D W T D L C L P A W G C L W D T E |
| 81 | 252 | E E W E D L C L P R W G C L W S A E |
| 11 | 253 | E F W Q D I C L P N W G C L W A E S |
| 24 | 254 | E G F S D I C L P R W G C L W S Q E |
| 93 | 255 | E T W E D L C L P N W G C L W D L E |
| 23 | 256 | G E V N D F C L P R W G C L W E G D |
| 33 | 257 | G G E W D V C L P A W G C L W G E E |
| 9 | 258 | K O W Y D I C L P R W G C L W G G E |
| 46 | 259 | K L G Q D I C L P R W G C L W D F A |
| 58 | 260 | L E E W D I C L P Q W G C L W R E G |
| 69 | 261 | L V L P D I C L P K W G C L W G D T |
| 21 | 262 | M D L A D I C L P K W G C L W E S D |
| 12 | 263 | M V L D D I C L P R W G C L W S E K |
| 57 | 264 | M W S G D L C L P R W G C L W G E T |
| 60 | 265 | N R M G D I C L P R W G C L W D G H |
| 72 | 266 | R D W E D L C L P N W G C L W E L S |
| 10 | 267 | R G D W D L C L P K W G C L W E G V |
| 47 | 268 | R Q W E D I C L P R W G C L W G V G |
| 94 | 269 | R V E Y D L C L P R W G C L W E P P |
| 36 | 270 | S I W S D I C L P R W G C L W E S D |
| 71 | 271 | T D E W D I C L P N W G C L W E A G |
| 95 | 272 | T E D V D F C L P L W G C L W E E P |
| 22 | 273 | V K E E D F C L P R W G C L W E A G |
| 48 | 274 | W D F E D I C L P R W G C L W A D M |
| 84 | 275 | W E D W D V C L P R W G C L W G G G |
| 59 | 276 | Y E D I D I C L P R W G C L W D L S |

TABLE 5

Sequences Selected on Rabbit Albumin

| | |
|---|---|
| Clone ID | Hard Randomization Library |

| | | |
|---|---|---|
| | 155 | X X X X X D X C L P X W G C L W X X X X |
| 75 | 277 | A G L D E D I C L P R W G C L W G K E A |
| 39 | 278 | A G M M G D I C L P R W G C L W Q G E P |
| 76 | 279 | A P G D W D F C L P K W G C L W D D D A |
| 74 | 280 | A Q L F D D I C L P R W G C L W S D G Y |
| 86 | 281 | A R T M G D I C L P R W G C L W G A S D |
| 63 | 282 | A W Q D F D V C L P R W G C L W E P E S |
| 26 | 283 | D T T W G D I C L P R W G C L W S E E A |
| 4 | 284 | E G F L G D I C L P R W G C L W G H Q A |
| 2 | 285 | E Q W L H D I C L P K W G C L W D D T D |
| 61 | 286 | E T G W P D I C L P R W G C L W E E G E |
| 52 | 287 | F E L G E D I C L P R W G C L W E E H N |
| 38 | 288 | G A S L G D I C L P R W G C L W G P E D |
| 88 | 289 | G E W W E D I C L P R W G C L W G S S S |
| 1 | 290 | G S L E S D I C L P R W G C L W G I D E |
| 13 | 291 | G W L E E D I C L P K W G C L W G A D N |
| 64 | 292 | H E Q W D D I C L P R W G C L W G G S Y |
| 49 | 293 | Q R V D D D I C L P R W G C L W G E N S |
| 50 | 294 | S V G W G D I C L P K W G C L W A E S D |
| 40 | 295 | T L M S N D I C L P R W G C L W D E P K |
| 28 | 296 | T L V L D D I C L P R W G C L W D M T D |
| 14 | 297 | T W Q G E D I C L P R W G C L W D T E V |
| 73 | 298 | V G V F D D I C L P R W G C L W E Q P V |
| 25 | 299 | V P A M G D I C L P R W G C L W E A R N |
| 16 | 300 | V S L G D D I C L P K W G C L W E P E A |
| 15 | 301 | V W I D R D I C L P R W G C L W D T E N |
| 51 | 302 | W R W N E D I C L P R W G C L W E E E A |
| 73 | 303 | A V S W A D I C L P R W G C L W E R A D |
| 37 | 304 | A W L D E D I C L P K W G C L W N T G V |
| 16 | 305 | F S L D E D I C L P K W G C L W G A E K |
| 3 | 306 | G D L G D D I C L P R W G C L W D E Y P |
| 87 | 307 | G E G W S D I C L P R W G C L W A E D E |
| | 155 | X X X X X D X C L P X W G C L W X X X X |
| 38 | 308 | G L M G E D I C L P R W G C L W K G D I |
| 75 | 309 | G W H D R D I C L P R W G C L W E Q N D |
| 63 | 310 | L L G G H D I C L P R W G C L W G G D V |
| 64 | 311 | M R W S S D I C L P K W G C L W G D E E |
| 13 | 312 | Q F E W D D I C L P R W G C L W E V E V |
| 49 | 313 | Q G W W H D I C L P R W G C L W E E G E |
| 51 | 314 | R E G W P D I C L P R W G C L W S E T G |
| 40 | 315 | R E L W G D I C L P R W G C L W E H A T |
| 76 | 316 | R L E L M D I C L P R W G C L W D P Q D |
| 2 | 317 | S G V L G D I C L P R W G C L W E E A G |
| 14 | 318 | S L G L T D L C L P R W G C L W E E E Q |
| 27 | 319 | S S L E Q D I C L P R W G C L W G Q D A |
| 74 | 320 | S V S S D D I C L P R W G C L W W D F S |
| 15 | 321 | T S L L D D I C L P R W G C L W Y E E G |
| 50 | 322 | T S L A D D I C L P R W G C L W S E D G |
| 25 | 323 | V E M W H D I C L P R W G C L W D S N A |
| 4 | 324 | W D L A S D I C L P R W G C L W E E E A |
| 40 | 325 | F I T Q D I C L P R W G C L W G E N |
| 13 | 326 | F L W R D I C L P R W G C L W S E G |
| 50 | 327 | F V H E D I C L P R W G C L W G E G |
| 26 | 328 | G L G D D I C L P R W G C L W G R D |
| 63 | 329 | G M F D D I C L P K W G C L W G L G |
| 37 | 330 | G P G W D I C L P R W G C L W G E E |
| 87 | 331 | G P W Y D I C L P R W G C L W D G V |
| 43 | 332 | G W D D D I C L P R W G C L W G D G |
| 39 | 333 | L E Y E D I C L P K W G C L W G G E |
| 14 | 334 | L L D E D I C L P R W G C L W G V R |
| 28 | 335 | L M S P D I C L P K W G C L W E G D |
| 52 | 336 | L V L G D I C L P R W G C L W E S D |
| 75 | 337 | M L S R D I C L P R W G C L W E E E |
| 61 | 338 | M P W T D I C L P R W G C L W S E S |
| 25 | 339 | R L G S D I C L P R W G C L W G A G |
| 51 | 340 | R L G S D I C L P R W G C L W D Y Q |
| 49 | 341 | S P W M D I C L P R W G C L W E S G |
| | 155 | X X X X X D X C L P X W G C L W X X X X |
| 38 | 342 | S T F T D I C L P R W G C L W E L E |
| 74 | 343 | S V L S D I C L P R W G C L W E E S |
| 86 | 344 | T W F S D I C L P R W G C L W E P G |
| 88 | 345 | V H Q A D I C L P R W G C L W G D T |
| 1 | 346 | V L L G D I C L P L W G C L W G E D |
| 15 | 347 | V N W G D I C L P R W G C L W G E S |
| 76 | 348 | V V W S D I C L P R W G C L W D K E |
| 73 | 349 | V W Y K D I C L P R W G C L W E A E |
| 85 | 350 | W D Y G D I C L P R W G C L W E E G |
| 2 | 351 | W E V Q D I C L P R W G C L W G D D |
| 27 | 352 | Y I W R D I C L P R W G C L W E G E |
| 3 | 353 | Y R D Y D I C L P R W G C L W D E R |
| 64 | 354 | A F W S D I C L P R W G C L W E E D |
| 49 | 355 | D W G R D I C L P R W G C L W D E E |
| 28 | 356 | E A W G D I C L P R W G C L W E L E |
| 61 | 357 | L E L S D I C L P R W G C L W D D T |
| 25 | 358 | L K L E D I C L P R W G C L W G E S |
| 52 | 359 | L L T R D I C L P K W G C L W G S D |
| 4 | 360 | L R W S D I C L P R W G C L W E E T |
| 87 | 361 | L Y L R D I C L P K W G C L W E A D |
| 76 | 362 | N W Y D D I C L P R W G C L W D V E |
| 1 | 363 | Q D W E D I C L P R W G C L W G D — |
| 38 | 364 | Q S W P D I C L P K W G C L W G E G |
| 88 | 365 | T L L Q D I C L P R W G C L W E S D |
| 74 | 366 | V R L M D I C L P R W G C L W G E E |
| 26 | 367 | V R W E D I C L P R W G C L W G E E |
| 40 | 368 | W D V A D I C L P R W G C L W A E D |
| 15 | 369 | W H M G D I C L P R W G C L W S E V |
| 14 | 370 | W K D F D I C L P R W G C L W D D H |
| 3 | 371 | W L S E D I C L P Q W G C L W E E S |
| 27 | 372 | W L S E D I C L P R W G C L W A A D |
| 37 | 373 | W L S D D I C L P R W G C L W D D L |

TABLE 6

Sequences Selected on Human Albumin

| | |
|---|---|
| Clone ID | Hard Randomization Library |

| | | |
|---|---|---|
| | 155 | X X X X X D X C L P X W G C L W X X X X |
| 68 | 374 | E V R E W D I C L P R W G C L W E N W R |
| 6 | 375 | F G W E W D I C L P R W G C L W G N E Q |
| 17 | 376 | I W G L E D I C L P R W G C L W E D G L |
| 53 | 377 | N T P T Y D I C L P R W G C L W G D V P |
| 5 | 378 | Q P V W S D I C L P R W G C L W G E D H |
| 18 | 379 | S E W G G D I C L P — W G C L W S E E S |
| 80 | 380 | W G M A R D W C L P M W G C L W R G G G |
| 7 | 381 | W H L T D D I C L P R W G C L W G D E Q |
| 67 | 382 | N W A E N D I C L P R W G C L W G D E N |
| 68 | 383 | S A R E W D I C L P T W G C L W E K D I |
| | 156 | X X X X D X C L P X W G C L W X X X |
| 42 | 384 | A G E W D I C L P R W G C L W D V E |
| 56 | 385 | E I R W D F C L P R W G C L W D E D |
| 8 | 386 | E S L G D I C L P R W G C L W G S G |
| 30 | 387 | E Y W G D I C L P R W G C L W D W Q |
| 80 | 388 | K M W S D I C L P R W G C L W E E E |
| 90 | 389 | M G T K D I C L P R W G C L W A E A |
| 7 | 390 | M H E W D I C L P R W G C L W E S S |
| 78 | 391 | R G L H D A C L P W W G C L W A G S |
| 19 | 392 | R L F G D I C L P R W G C L W Q G E |
| 5 | 393 | S G E W D I C L P R W G C L W G E G |
| 6 | 394 | S M F F D H C L P M W G C L W A E Q |
| 44 | 395 | V G E W D I C L P N W G C L W E R E |
| 32 | 396 | W W M A D R C L P L W G C L W R G D |
| 29 | 397 | W W V R D L C L P T W G C L W S G K |
| 54 | 398 | Y F D G D I C L P R W G C L W G S D |
| 32 | 399 | T L F Q D I C L P R W G C L W E E S |
| 68 | 400 | W F P K D R C L P V W G C L W E R H |

Peptide Synthesis

Peptides were synthesized by either manual or automated (Milligen 9050) Fmoc-based solid phase synthesis on a 0.25 mmol scale using a PEG-polystyrene resin (Bodanszky M., 1984, In: Principles of Peptide Synthesis, Springer, Berlin; Dennis et al., 2001 Biochemistry 40: 9513-21). Side chain protecting groups were removed and the peptides were cleaved from the resin with 95% trifluoroacetic acid (TFA) and 5% triisopropylsilane. A saturated iodine solution in acetic acid was added for oxidation of disulfide bonds. Peptides were purified by reversed phase HPLC using a water/acetonitrile gradient containing 0.1% TFA. Peptides were more than 95% pure by analytical HPLC. Identity was verified by mass spectrometry.

The carboxy terminal lysine of peptide SA08 was derivatized with NHS-LC-biotin as recommended by the manufacture (Pierce Chemical, Rockford, Ill.) and purified by HPLC as above to yield SA08b: Ac-QGLIGDICLPRWGCLWGDSVK_{b}-NH2 (SEQ ID NO: 12), where K_{b} refers to lysine-biotin.

SA08b Binding Assay

Rabbit, rat, or mouse albumin was immobilized directly on Maxisorp plates at 10 μg/ml in PBS, overnight at 4° C. Plates were blocked as described above. Serially diluted samples were suspended in binding buffer (above) and added to the plate followed by the addition of 10 nM SA08b for 1 hour, at 25° C. The microtiter plate was washed with PBS, 0.05% Tween 20 and SA08b bound to albumin was detected with Streptavidin/HRP. The amount of HRP bound was measured using ABTS/H₂O₂ substrate and monitoring the change at 405 nm.

Peptides corresponding to identified phage sequences were synthesized and their affinity for rat, rabbit or mouse albumin measured using the SA08b binding assay. Binding affinity data are shown below in Table 7.

A series of peptides having the core sequence, DICLPRWGCLW (SEQ ID NO:8), was identified. These peptides specifically bind albumin from multiple species with high affinity, and bind to albumin with a 1 to 1 Stochiochemistry, at a site district from that of known small molecule binding sites.

TABLE 7

Peptides Binding Multiple Species Albumin

IC₅₀ (nM)

| Peptide | ID | | Rabbit | Rat | Mouse |
|---|---|---|---|---|---|
| SA02 | 7 | D L C L R D W G C L W -n | | | |
| SA04 | 8 | D I C L P R W G C L W -n | 8543 | 787 | 40 |
| SA05 | 16 | M E D I C L P R W G C L W E D -n | 804 | 161 | 6 |
| SA06 | 401 | Q R L M E D I C L P R W G C L W E D D F -n | 128 | 68 | 8 |
| SA07 | 11 | Q G L I G D I C L P R W G C L W G D S V -n | 30 | 35 | 6 |
| SA08 | 12 | Ac Q G L I G D I C L P R W G C L W G D S V K -n | 63 | 68 | 10 |
| SA09 | 13 | Ac E D I C L P R W G C L W E D D -n | 1687 | 258 | 6 |
| SA10 | 14 | Ac R L M E D I C L P R W G C L W E D D -n | 86 | 77 | 4 |
| SA11 | 15 | Ac M E D I C L P R W G C L W E D D -n | 1213 | 232 | 17 |
| SA12 | 16 | Ac M E D I C L P R W G C L W E D -n | 1765 | 205 | 13 |
| SA13 | 17 | Ac R L M E D I C L A R W G C L W E D D -n | 3200 | 2480 | 188 |
| D3H44-L | 401 | Q R L M E D I C L P R W G C L W E D D F -n | 241 | | |
| S3H44-Ls | 401 | Q R L M E D I C L P R W G C L W E D D F -n | 75 | | |

Affinity Measurements by Surface Plasmon Resonance

Binding affinities between SA peptides and album were obtained using a BIAcore 3000 (BIAcore, Inc., Piscataway, N.J.). Albumin was captured in a CM5 chip using amine coupling at approximately 5000 resonance units (RU). SA peptides (0, 0.625, 1.25, 2.5, 5, and 10 μM were injected at a flow rate of 20 μl/minute for 30 seconds. The bound peptides were allowed to disassociate for 5 minutes before matrix regeneration using 10 mM glycine, pH 3.

The signal from an injection passing over an uncoupled cell was subtracted from that of an immobilized cell to generate sensongrams corresponding to the amount of peptide bound as a function of time. The running buffer, PBS containing 0.05% TWEEN-20T, was used for all sample dilutions. BIAcore kinetic evaluation software (v 3.1) was used to determine the dissociation constant (K_{d}) from the association and dissociation rates, using a one to one binding model.

The affinity of selected peptides for binding human (HAS), rabbit (BuSA), rat (RSA), and mouse (MSA) albumin was assessed by the BIAcore assay as well as SA08 peptide competition assay. The data, shown below in Table 8, demonstrate that the IC₅₀ values obtained in the competition assay compared favorably with the K_{d} values obtained in the BIAcore assay. Peptide SA15, representing the consensus peptide for binding rabbit albumin, had the lowest IC₅₀ value in the competition assay and the highest affinity by surface plasmon resonance for rabbit albumin. A linear peptide, identical to SA06, but having both Cys residues altered to Ala, had an IC₅₀ that was greater than 50 μM, demonstrating the importance of the disulfide.

TABLE 8

| | |
|---|---|
| | Peptide |
| Surface Plasmon Resonance | Competition |
| Kd (nM) | IC₅₀ (nM) |

| | | | | | | | |
|---|---|---|---|---|---|---|---|
| HuSA | BuSA | RSA | SA | ID | SEQUENCE | BuSA | MuSA |

| | | | | | | | |
|---|---|---|---|---|---|---|---|
| 467 ± 47 | 320 ± 22 | 266 ± 6 | 21 | 402 | _{Ac-}RLIEDICLPRWGCLWEDD_{-NH2} | 270 ± 110 | 7 ± 2 |
| 803 ± 82 | 143 ± 5 | 229 ± 9 | 06 | 403 | QRLMEDICLPRWGCLWE | 130 ± 50 | 6 ± 2 |
| 858 ± 59 | 108± | 158 ± 3 | 08 | 11 | _{Ac-}QGLIGDICLPRWGCLWGDSVK_{-NH2} | 51 ± 11 | 12 ± 2 |
| 878 ± 58 | 65 ± 3 | 150 ± 5 | 15 | 404 | GEWWEDICLPRWGCLWEEED_{-NH2} | 13 ± 2 | 5 ± 1 |

In addition, the peptide affinity for rabbit albumin diminished with reduction in the length of the peptides, as shown below in Table 9. The binding affinity of a core sequence of about 10 amino acids (SA34 and SA19) having IC₅₀ values of approximately 25 μM was improved approximately 6-fold by the addition of 4 residues to the amino terminus (SA33) or about 8.6-fold by the addition of three residues to the carboxy terminus (SA26). The addition of 7 residues, 4 to the amino terminus and 3 to the carboxy terminus, resulted in a 60-fold improvement in the IC₅₀ (SA22).

When the binding of a RB-B8 or RB-Hi phage to rabbit albumin was monitored over a pH range from 2.9 to 9.0, optimum binding was observed above pH6 for both clones (data not shown). Binding decreased below pH6.0 until no binding was observed at pH 2.9. A similar pattern was observed for the binding of these clones to human and rat albumin. The similar amino acid preferences and pH profiles are consistent with a similar binding environment on each species of albumin.

Since albumin plays an important role as a carrier of many ligands and drugs, known albumin ligands were analyzed for ability to compete with peptide binding. The addition of site I ligands (indomethacin, phenylbutazone, warfarin) or site II ligands (ibuprofen, L-tryptophan, dansylsarcosine, diazepam)m a fatty acid (myristic acid) or a metal ion (CuCl₂) at concentrations of up to 100 μM had no effect on SA08b peptide binding to rat or rabbit albumin in the peptide competition assay (data not shown).

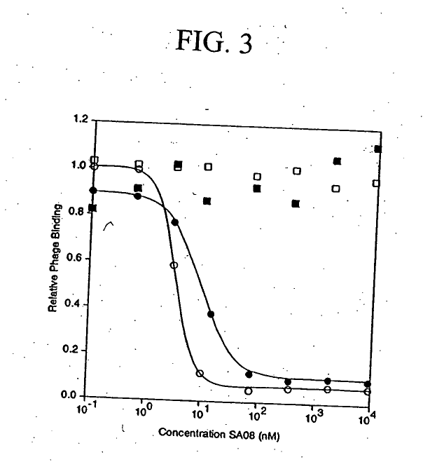

Unrelated clones that were initially identified for binding to albumin were also tested for competition with the matured, multi-species binding peptides. While RD and BA phage selectively bound only to rat and rabbit albumin, respectively, these clones were clearly blocked by the addition of the binding peptide SA08 (FIG. 3), demonstrating binding to a different site on albumin.

TABLE 9

| | | | |
|---|---|---|---|
| PEP- | | | |
| TIDE | ID | SEQUENCE | IC₅₀ (nM) |

| | | | |
|---|---|---|---|
| SA20 | 405 | _{Ac} QRLIEDICLPRWGCLWEDDF _{NH2} | 260 |
| SA21 | 402 | _{Ac} RLTEDICLPRWGCLWEDD _{NH2} | 270 ± 110 |
| SA22 | 406 | _{Ac} RLTEDICLPRWGCLWED _{NH2} | 430 ± 70 |
| SA29 | 407 | _{Ac} RLWDICLPRWGCLWE _{NH2} | 400 ± 90 |
| SA31 | 408 | _{Ac} RLIEDICLPRWGCLW _{NH2} | 200 |
| SA33 | 409 | _{Ac} RLIEDICLPRWGCL _{NH2} | 4310 ± 2770 |
| SA35 | 410 | _{Ac} RLIEDICLPRWGC _{NH2} | >250000 |
| SA23 | 411 | _{Ac} LIEDICLPRWGCLWED _{NH2} | 360 ± 140 |
| SA24 | 412 | _{Ac} IEDICLPRWGCLWED _{NH2} | 1380 ± 410 |
| SA25 | 413 | _{Ac} EDICLPRWGCLWED _{NH2} | 2730 ± 1300 |
| SA26 | 414 | _{Ac} DICLPRWGCLWED _{NH2} | 3120 ± 660 |
| SA27 | 415 | _{Ac} ICLPRWGCLWED _{NH2} | 86700 ± 21800 |
| SA28 | 416 | _{Ac} CLPRWGCLWED _{NH2} | >400000 |
| SA30 | 417 | _{Ac} IEDICLPRWGCLWE _{NH2} | 1800 ± 590 |
| SA32 | 418 | _{Ac} EDICLPRWGCLW _{NH2} | 2170 ± 520 |
| SA04 | 8 | DICLPRWGCLW _{NH2} | 8540 ± 4620 |
| SA34 | 419 | _{Ac} DICLPRWGCL _{NH2} | 28210 ± 6500 |
| SA19 | 419 | DICLPRWGCL _{NH2} | 24510 ± 2100 |
| SA18 | 420 | ICLPRWGCLW _{NH2} | 124900 |
| SA36 | 421 | _{Ac} ICLPRWGC _{NH2} | >250000 |

### EXAMPLE 2

### Albumin Binding Fab Fusions

Construction, Expression and Purification of Albumin Binding Fab Fusions

Compared to an IgG, Fab fragments have a relatively fast clearance rate (42-72 ml/kg/hour in rabbit) (Timsina et. al., 1990, J. Pham Pharmacol 42:572-6). In order to test whether association with albumin could increase the half-life of proteins and peptides in vivo, the sequence of SA06 was fused to a Fab fragment (D3H44) directed for binding tissue factor (TF). D3H44 is a humanized antibody that binds to human tissue factor (TF) and acts as an anticoagulant.

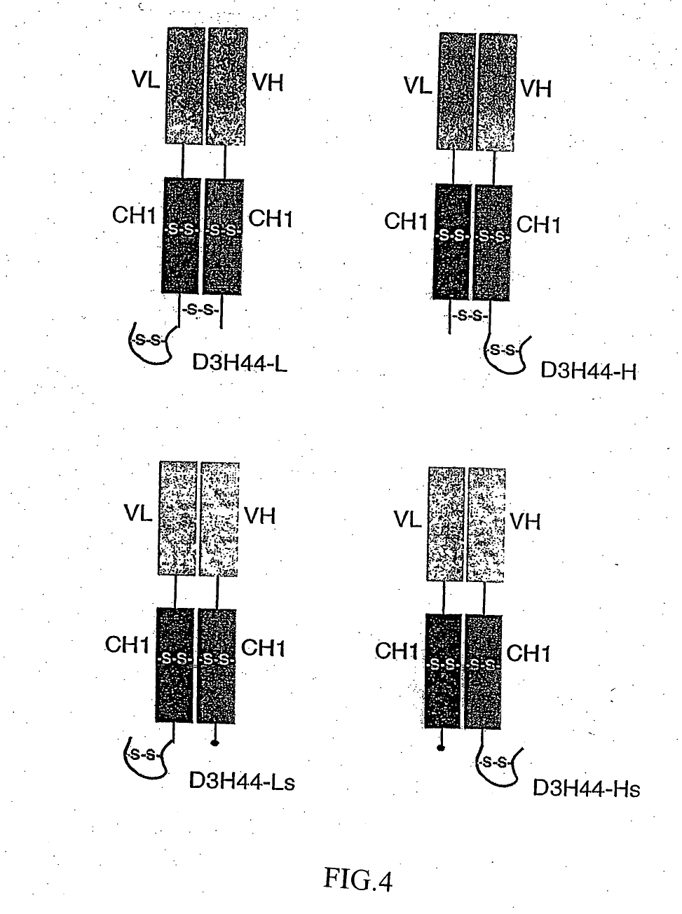

D3H44 Fab was produced as described in Presta et al., 2001, Thromb, Haemost 85:379-89. The SA06 sequence (QRLMED1CLPRWGCLWEDDF) (SEQ ID NO:401) was added to the carboxy terminus of either the light chain of the Fab to yield D3H44L or to the heavy chain of the Fab to yield D3H44H, via an inserted linker moiety, (GGGS) (SEQ ID NO:422) using Kunkel mutagenesis (Kunkel et al., 1987, Methods Enzym 154: 367-382). In addition, as a precaution against folding problems, an identical construction was made but with the intra-chain disulfide replaced by alanines (D3H44-Ls and D3H44-Hs, respectively) as depicted in FIG. 4. The plasmids were confirmed by sequencing.

The fusions were expressed under control of the alkaline phosphatase promoter and secreted from E. coli using the stII secretion signal. Fab fusions were recovered from the periplasm by suspending cells in 1 mM EDTA, 10 mM Tris-HCl, pH8, for 1 hour at 4° C. Cell debris was removed by centrifugation and the anti-TF Fab was selectively purified using a Hi-Trap (Amersham Pharmacia Biotech, Piscataway, N.J.) TF affinity column. Properly folded D3H44-L or D3H44-Ls was further purified using a rabbit albumin affinity column (rabbit albumin coupled to CNBr-activated Sepharose 4B, Amersham Pharmacia Biotech, Piscataway, N.J.). Both columns were washed with PBS and eluted with 50 mM HCl. Eluted fractions were neutralized with 1 M Tris pH 8. Endotoxin was further removed following extraction with Triton X114, as described in Aida and Pabst, 1990 J. Immunol. Methods 132:191. Thus the addition of SA06 provided a simple purification scheme using a TF affinity column followed by an albumin affinity column.

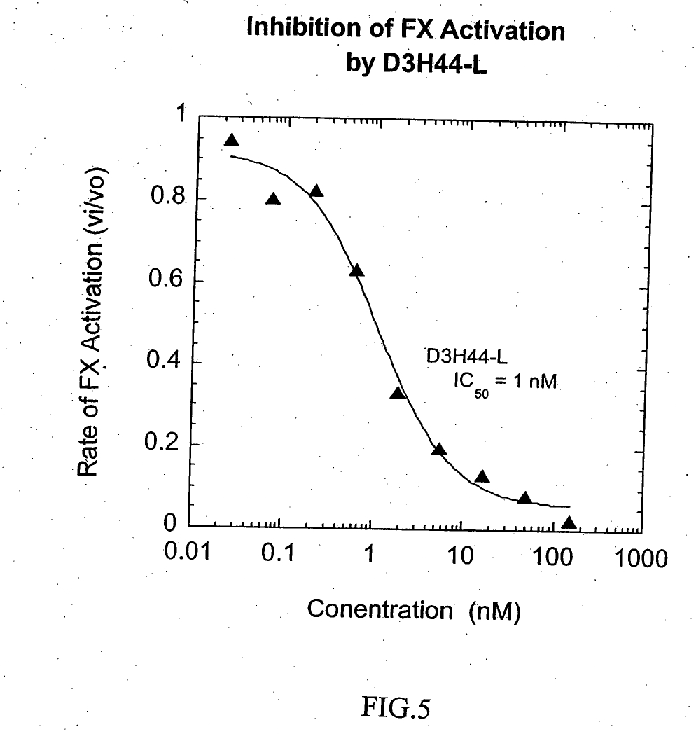

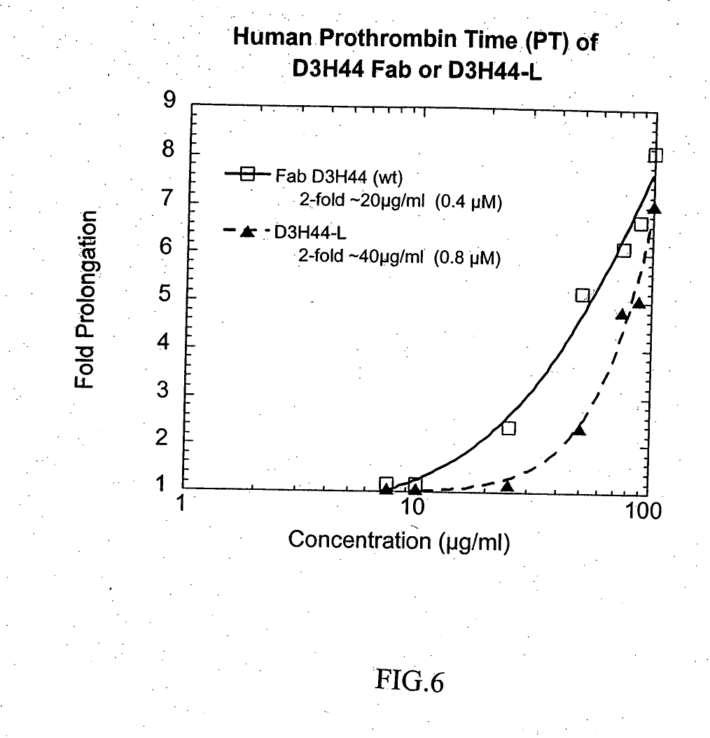

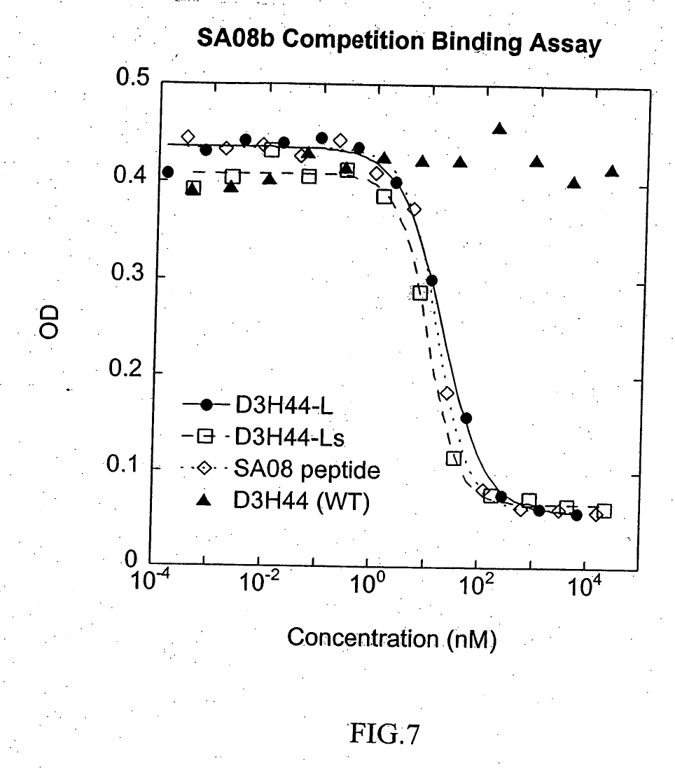

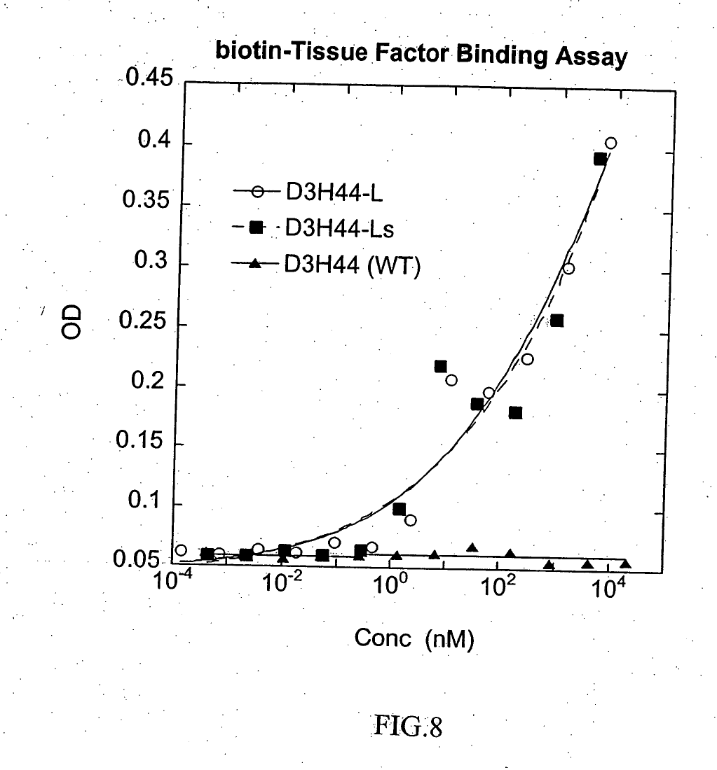

Purified D3H44 fusions retained their ability to bind TF as measured using a FX activation assay (FIG. 5), and a prothrombin time assay that measures prolongation of tissue factor dependent clotting (FIG. 6). The assay methods are described, for example, in Dennis et al., 2000, Nature 404: 465. As shown in FIG. 7, D3H44-L and D3H44-Ls each bound to albumin in the SA08b binding assay, unlike the wild type D3H44 that did not contain the albumin binding sequence (WT). Further, each of the albumin-binding fusions bound TF and albumin simultaneously. Simultaneous binding was demonstrated in a biotin-TF binding assay. In this assay, binding of the D3H44 fusions to immobilized albumin was detected with biotinylated TF. Wild-type D3H44 (WT), lacking the albumin binding peptide, did not bind albumin and thus did not generate a signal upon addition of biotinylated TF (FIG. 8).

Pharmacokinetics of D3H44 Albumin-Binding Fusions

D3H44 variants were administered to rabbits as a 0.5 mg/kg bolus injection into the marginal ear vein. Each test group consisted of 3 rabbits (5 rabbits were used in the F(ab′)2 group). Serum samples were taken at the indicated time points, serially diluted, and the concentration of D3H44 was determined using a TF binding ELISA.

Pharmacokinetic analysis was performed using the test article plasma concentrations. Group mean plasma data for each test article conforms to a multi-exponential profile when plotted against the time post-dosing. The data were fit by a standard two-compartment model with bolus input and first-order rate constants for distribution and elimination phases. The general equation for the best fit of the data for i.v. administration was: c(t)=Ae^{-αt}+Be^{βt}, where c(t) is the plasma concentration at time t, A and B are intercepts on the Y-axis, and α and β are the apparent first-order rate constants for the distribution and elimination phases, respectively. The α-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(α−k21)/(α−β), B=D/V (β−k21)/(α−β), and α and β (for α>β) are roots of the quadratic equation: r²+(k12+k21+k10)r+k21k10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles were made using KaleidaGraph (KaleidaGraph™ V. 3.09 Copyright 1986-1997. Synergy Software. Reading, Pa.). Values reported as less than reportable (LTR) were not included in the PK analysis and are not represented graphically. Pharmacokinetic parameters were determined by compartmental analysis using WinNonlin software (WinNonlin® Professional V. 3.1 WinNonlin™ Copyright 1998-1999. Pharsight Corporation. Mountain View, Calif.). Pharmacokinetic parameters were computed as described in Ritschel W A and Kearns G L, 1999, IN: Handbook Of Basic Pharmacokinetics Including Clinical Applications, 5th edition, American Pharmaceutical Assoc., Washington, D.C.

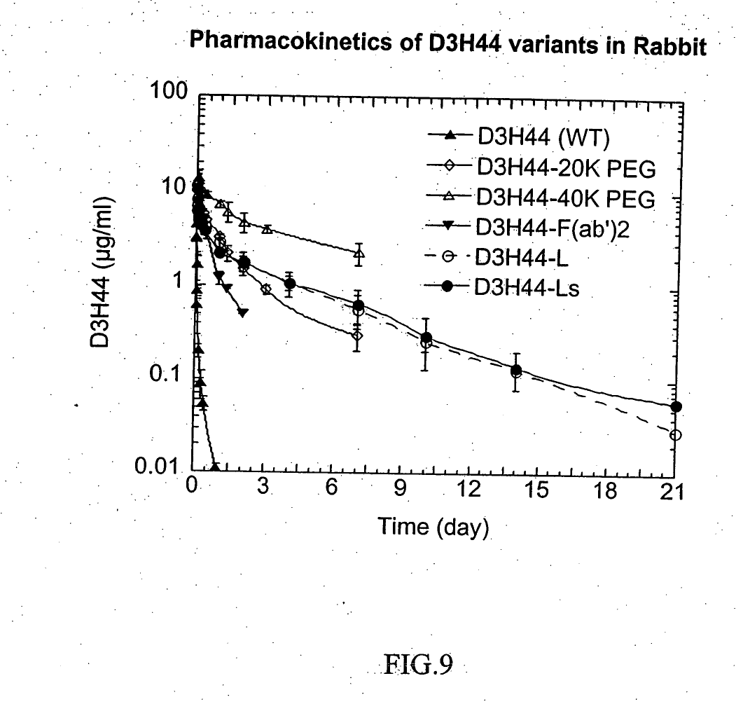

Fusion of the albumin binding peptide to D3H44 resulted in a protein having improved pharmacokinetic parameters, as demonstrated by the data shown in FIG. 9 and in Table 10, below. D3H44-L displayed a 70-fold increase in half-life (K10-HL) relative to the wild-type Fab, and a comparable half-life to D3H44 Fabs derivatized with 20K or 40K polyethylene glycol (PEG).

TABLE 10
Summarized PK Data

| | | |
|---|---|---|
| D3H44-Fab | D3H44-L | D3H44-Ls |

| Parameter | Units | Avg | SD | Avg | SD | Avg | SD |
|---|---|---|---|---|---|---|---|
| Dose | ug/kg | 396 | | 416 | | 524 | |
| AUC | hr*ug/mL | 5.86 | 1.23 | 349 | 33 | 332 | 82 |
| AUC/Dose | (hr*ug/mL)/(mg/kg) | 14.8 | 3.1 | 840 | 78 | 633 | 157 |
| CL | mL/hr/kg | 69.9 | 16.2 | 1.20 | 0.11 | 1.64 | 0.37 |
| Cmax | ug/mL | 5.00 | 2.30 | 7.55 | 1.23 | 5.98 | 0.11 |
| K10-HL | Hr | 0.876 | 0.213 | 32.4 | 3.2 | 38.3 | 8.8 |
| MRT | Hr | 3.07 | 0.62 | 95.0 | 13.1 | 110 | 20 |
| V1* | mL/kg | 90.6 | 38.4 | 56.2 | 10.1 | 87.6 | 1.7 |
| Vss* | mL/kg | 221 | 95 | 113 | 7 | 176 | 11 |

Summarized PK Data (Historical)

| | | D3H44-20kPEG | | D3H44-40kPEG | | D3H44-Fab | | D3H44-Fab′2 | |
|---|---|---|---|---|---|---|---|---|---|
| Parameter | Units | Avg | SD | Avg | SD | Avg | SD | Avg | SD |
| AUC | hr*ug/mL | 271 | 33 | 1255 | 383 | 9.8 | 1.6 | 120 | 13 |
| CL | mL/hr/kg | 1.87 | 0.23 | 0.422 | 0.119 | 51.8 | 9.2 | 4.21 | 0.51 |
| K10-HL | hr | 18.0 | 4.2 | 68.9 | 28.5 | 0.760 | 0.123 | 8.84 | 0.73 |
| V1 | ug/mL | 47.4 | 5.4 | 39.2 | 7.4 | 55.9 | 4.7 | 53.5 | 5.3 |
| Vss | mL/kg | 109 | 14 | 78.8 | 13.7 | 127 | 13 | 84.5 | 11.4 |
| Dose | ug/kg | 509 | | 493 | | 500 | | 500 | |
| AUC/Dose | (hr*ug/mL)/(mg/kg) | 532 | 65 | 2546 | 777 | 19.7 | 3.2 | 240 | 26 |

p < 0.05 RC20L vs RC20Ls
AUC = area under the curve
CL = clearance
K10-HL = half-life from compartment 1
MRT = mean residence time
V1 = initial distribution volume
Vss = distribution volume at steady state

### EXAMPLE 3

### Albumin Binding Anti-HER Fab Fusions

The peptide ligand SA06, having the amino acid sequence: QRLMEDICLPRWGCLWEDDF (SEQ ID NO:401) was analyzed for binding activity against multiple species of albumin, using the competitive SA08b albumin binding assay described above for Example 1. As shown Table 11 below, the peptide ligand SA06 bound albumin with IC₅₀ values ranging from 5000 nM to 8 nM, depending on the species of albumin analyzed.

TABLE 11
Binding of SA06 to Albumin

| Albumin Species | IC₅₀ (nM) |
|---|---|
| Human | 5,000 |
| Rabbit | 128 |
| Rat | 68 |
| Mouse | 8 |

Fusion of SA06 to anti-HER2 Fab to form 4D5-H

The SA06 albumin binding peptide was fused recombinantly to fragments of an anti-HER2 antibody, the murine monoclonal antibody muMAb4D5 (herein 4D5). 4D5 is directed against the extracellular domain of p185^{HER2} (HER2). This antibody and its functional activities are described, for example, in Fendly et. al, 1990, Cancer Res. 50:1550-1558 and in published PCT application WO89/06692. The antibody is produced from hybridoma cells deposited with the American Type Culture Collection in Manassas, Va., and has ATCC accession number CRL 10463.

A 4D5 Fab fragment was fused to the SA06 albumin binding peptide to form the fusion peptide 4D5-H, using methods described above for the fusion of SA08 to the anti-TF antibody, D3H44. In brief, the polynucleotide sequence encoding the SA06 peptide was added to the polynucleotide sequence encoding the heavy chain of 4D5 at its carboxy terminus. The fusion peptide was expressed and secreted from E. coli, isolated, and purified according to the methods described above for Example 1. In a similar manner, a 4D5 diabody was also fused to the SA06 albumin binding peptide to form dia4D5-H.

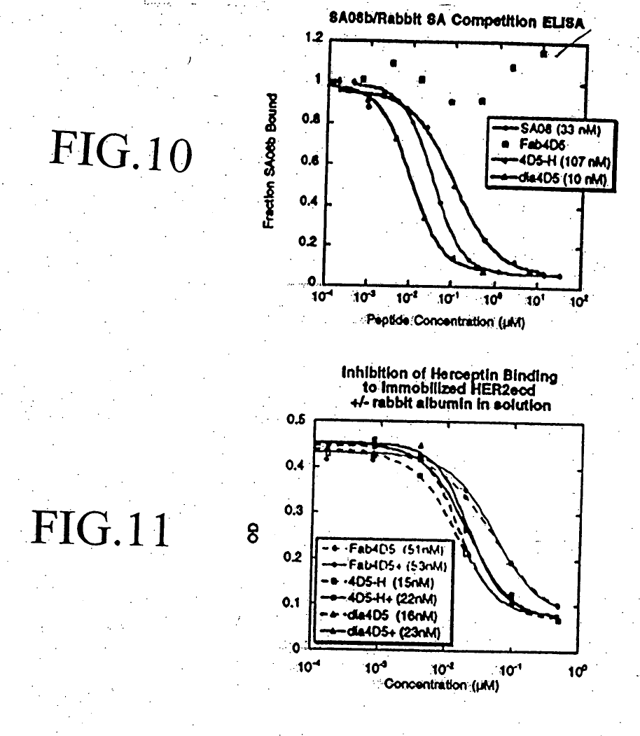

Binding of the fusions to albumin was analyzed, using the SA08b competition assay as described above for Example 1, and with the wild type Fab, 4D5 as control. Each of the peptide fusions bound albumin had an ability to compete for binding to immobilized albumin, in contrast to the WT Fab fragment, which was unable to bind albumin (FIG. 10).

4D5-H binds HER2

Purified 4D5-H and dia4D5-H fusions were analyzed for antigen-binding using an inhibition binding assay. The ability of the fusions to inhibit binding of Herceptin™ to immobilized antigen, HER2 was analyzed in the presence and absence of rabbit albumin. Herceptin binding to HER2 was inhibited by the fusion peptides 4D5-H and dia4D5-H in the presence and absence of albumin in the reaction solution (FIG. 11).

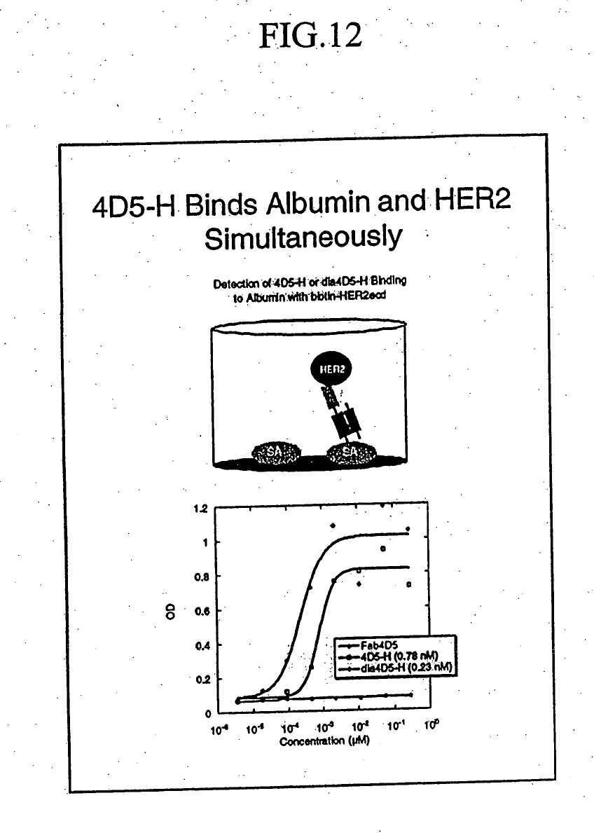

To further characterize the binding of the fusion constructs, the HER2 antigen was labeled with biotin. Briefly, the fusion construct was added to albumin coated microtiter plates. Biotinylated HER2 antigen was added to the solution, and incubated. The plates were washed and analyzed for bound biotin. As shown in FIG. 12, the 4D5-H and dia4D5-H fusions bound the immobilized albumin and the free HER2 antigen simultaneously, whereas the Fab4D5 failed to bind albumin, and was not detected.

### EXAMPLE 4

### Tumor Targeting with 4D5-H

The monovalent 4D5 Fab fragment (4D5 Fab, 50 kDa, (Kelley, R. F., et al., Biochemistry 31:5434-5441 (1992))) and SA06-Fab fusion 4D5-H (Fab-H) (52 kDa), and the bivalent IgG Herceptin™ (155kDa) were each labeled with Cy3. These molecules were each shown to stain HER2 positive tumors in vitro.

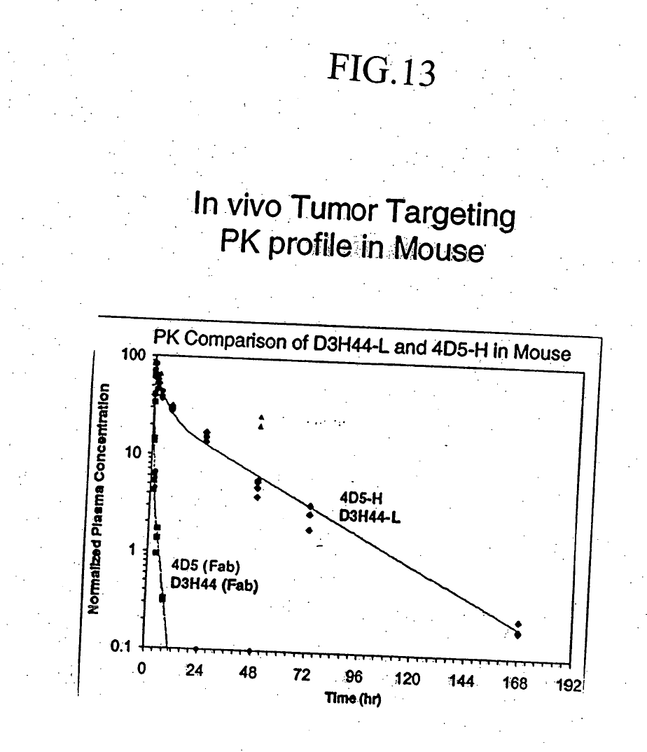

To examine the utility of the fusions in vivo, nude mice bearing HER2-positive tumors (MMTV-HER2 F05) were administered equivalent doses of the 4D5 (IgG), 4D5-H (Fab-H), and the Fab fragment, Herceptin®. Plasma concentration of the administered drug was analyzed to determine the in vivo tumor targeting PK profiles in the mice. As shown in FIG. 13, the normalized plasma concentration of the fusion 4D5-H was sustained over time as compared with that of the Fab 4D5.

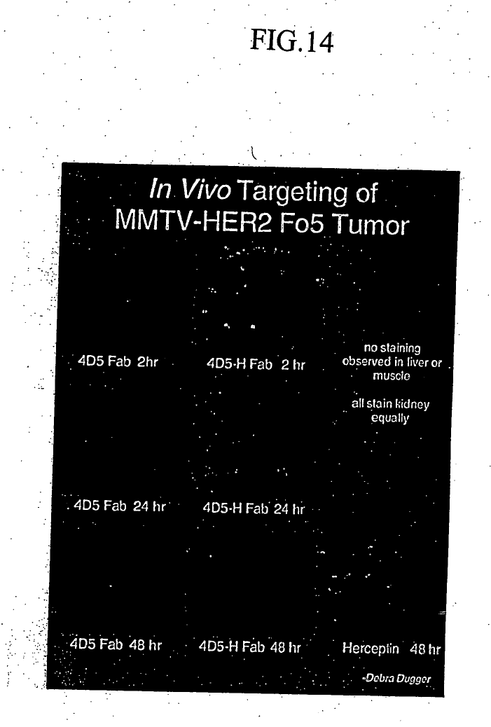

At 2, 24, and 48 hours after administration of the peptides, tumor samples were taken and analyzed for the presence of the Cy3-labeled peptides. Tumor histology (FIG. 14) surprisingly demonstrated staining of tumors within 2 hours for the 4D5-H treated animals, with diffuse staining through the tumor present at 24 hours post administration. At 24 hours, significant staining by the fusion was seen in the tumor. At 48 hours, little staining remained. In tumors taken from animals receiving the IgG, Herceptin, staining was less rapid, and peaked at 48 hours post-administration.

The data suggests a combination of factors, including the use of an albumin binding peptide and size of the therapeutic molecule may control uptake and retention of the therapeutic molecule by the tumor. The data herein demonstrates the improved pK profiles and tumor targeting of a therapeutic peptide via simple recombinant fusion that provides selective binding to albumin. The ability to effect these changes without a dramatic increase in the size presents an advantage for tumor targeting and imaging. A low mw agent has an advantage in tissue diffusion, however a sufficient time of exposure is needed for adequate absorption. Generally a small protein such as a scfv can diffuse rapidly into tissues but the bulk of the material is lost due to extremely fast renal filtration. In contrast, large IgG remains circulating several days, providing ample exposure but only minimal tissue (eg tumor) penetration due to poor diffusion. A small long-lived molecule, such as an albumin binding fab fusion provides a useful agent for tissue targeting, eg tumor targeting for therapy or imaging. The ability to modulate these pharmacokinetic properties by manipulation of the specific affinity of the peptide for albumin by sequence manipulation provides a unique method for providing tissue specific agents.

### EXAMPLE 5

### 4D5H Albumin Binding Fabs (AB.Fab) Variants Having Varied Affinity for Albumin

In the present example, preparation of serum albumin binding peptide variants of 4D5-H AB.Fab are disclosed having altered albumin binding affinity. Truncation of the albumin binding peptide was performed to generate multiple AB.Fab variants with different affinities for albumin. An assay to measure binding in solution was developed in order to better reflect their binding affinity for albumin in vivo. The pharmacokinetic parameters of the 4D5-H AB.Fab variants in animal studies were used in this Example to predict pharmacokinetic parameters of the 4D5H AB.Fab in humans.

Albumin binding affinities varied depending upon the species of albumin tested and values obtained for binding to soluble albumin differed from those determined when albumin was immobilized. The AB.Fab variants bound to soluble mouse, rat and rabbit albumin with affinities ranging from 40 nM to 2.5 μM. In both rats and rabbits, increased affinity for albumin correlated with reduced clearance and a prolonged half-life. Over the affinity range sampled, the beta half-life of the AB.Fab variants ranged 6-fold in rats and rabbits while their clearance ranged over 50 and 20-fold, respectively. Using this information and an allometric scaling for albumin, the beta half-life for AB.Fab4D5-H was estimated for humans as 4 days in humans with a clearance of 76 mL/h.

These AB.Fab variants demonstrate the ability to modulate the clearance of a Fab fragment in vivo and to predict pharmacodynamics in humans based on binding results in animal species.

Abbreviations used herein include the following: HER2, human epidermal growth factor receptor 2; HER2ecd, the extracellular domain of HER2; scFv, a single chain fusion of the light and heavy antigen binding domains of an IgG, Fab, the antigen binding fragment consisting of the light chain and the variable and first constant domains of the heavy chain; AB.Fab, albumin binding Fab; Fab4D5, the Fab portion of Herceptin® (trastuzumab), SA, serum albumin; ELISA, enzyme-linked immunosorbent assay; PBS, phosphate buffered saline; RU, response units, AUC, area under the concentration-time curve extrapolated to infinity; CL, clearance; t_{1/2}β, beta-half-life; V₁, volume of distribution of the central compartment; Vₛₛ, steady state volume of distribution It is disclosed herein that the clearance of a Fab fragment can be dramatically decreased through association with albumin (see also, Dennis, M. S., et al. (2002) J. Biol. Chem. 277, 35035-35043). The association with albumin was accomplished by the genetic fusion of an albumin-binding peptide sequence to the light chain of the Fab generating an albumin-binding Fab (AB.Fab) fully capable of binding antigen while bound to albumin. This albumin binding peptide, identified using peptide phage display, binds with a stoichiometry of 1:1 and at a site distinct from known small molecule ligand sites on albumin. This site is conserved among albumins from multiple species facilitating studies in many different animal models. The albumin binding affinity of the peptide varies however, as does the in vivo half-life of albumin among different animal species, thereby complicating the ability to predict pharmacokinetic properties from one species to another. There is a need to develop a means for making such predictions such as by using data from animal pharmacokinetic studies to predict pharmacokinetics in humans for a particular therapeutic Ab.Fab.

The albumin half-life in different species generally adheres to an allometric scaling based upon animal weight. For example, the half-life of albumin in mouse, rat, rabbit and man has been estimated as 1, 1.9, 5.55 and 19 d, respectively (Stevens, D. K. et al. (1992) Fundam. Appl. Toxicol. 19, 336-342; Reed, R. G., and Peters, T., Jr. (1984) Fed. Proc. 43, 1858; Hatton, M. W. C. et al. (1993) J. Theor. Biol. 161, 481-490; Sterling, K. (1957) J. Clin. Invest. 30, 1228-1237) and suggests the relationship:
Albumin half life (days)=3.75*Body Weight (kg)^{0.368}
assuming typical body weights of 0.02, 0.25, 3 and 70 kg, respectively. A method is disclosed herein which takes into account the affinity of an AB.Fab for albumin in one species relative to another and uses allometric scaling based on albumin to estimate the pharmacokinetic properties of an AB.Fab in humans. In addition, AB.Fab variants of 4D5-H are disclosed herein which have similar affinities for albumins from different species.

In this Example, the affinity of AB.Fab variants for albumin was altered by shortening the albumin-binding peptide so that destabilizing changes were avoided. In addition, an assay to measure the dissociation constant (Kd) of the AB.Fab variants for soluble albumin was used in order to accurately assess albumin binding in vivo. A direct correlation between albumin binding affinity and the pharmacokinetic attributes of the AB.Fab variants is disclosed herein which helps to define the affinity for albumin required to achieve a desired phamacokinetic profile. Taken together with the half-life of albumin among various animal species, the terminal half-life and clearance of an AB.Fab in humans can be estimated according to the methods disclosed herein. In addition, the 4D5-H AB.Fab and its albumin binding peptide variants disclosed herein can find use as therapeutic targeting molecules.

Procedures for Preparing 4D5-H Variants

Construction of AB.Fab variants - AB.Fab variants 4D5-H, 4D5-H4 and 4D5-H8 were constructed by digesting pAK19 (Carter, P. et al. (1992) Bio/Technology 10, 163-167; see also Example 1, above, for construction of AB.Fab 4D5-H) with Sal I and Sph I, and replacing this region with an annealed and ligated 4-oligonucleotide cassette. This cassette allowed the introduction of oligonucleotides encoding albumin binding peptide sequences of varied length to the carboxyl terminus of the Fab heavy chain four residues after the last cysteine in the constant domain (i.e. following the sequence: CDKTH (SEQ ID NO:428), Table 12). 4D5-H included a linker sequence (GGGS, SEQ ID NO:422) that was omitted in variants 4D5-H4, 4D5-H8, 4D5-H10, and 4D5-H11. AB. Fab variants 4D5-H10 and 4D5-H11 were constructed by introducing deletions into 4D5-H4 using QuikChange™ mutagenesis according to the manufacturer's instructions (Stratagene, La Jolla, Calif.).

TABLE 12

Albumin-binding peptide sequences added to the
carboxyl terminus of the heavy chain of Fab4D5
(ending CDKTH (SEQ ID NO:428)) to generate the
AB.Fab variants.

| | | albumin binding |
|---|---|---|
| AB.Fab | Linker | peptide sequence |
| 4D5-H | GGGS | QRLMEDICLPRWGCLWEDDF |
| | (SEQ ID NO:422) | (SEQ ID NO:401) |
| 4D5-H4 | None | DICLPRWGCLWED |
| | | (SEQ ID NO:414) |
| 4D5-H8 | None | IEDICLPRWGCLWE |
| | | (SEQ ID NO:417) |
| 4D5-H10 | None | DICLPRWGCLW |
| | | (SEQ ID NO:8) |
| 4D5-H11 | None | DICLPRWGCL |
| | | (SEQ ID NO:419) |

Pharmacokinetic studies in rat, rabbit and mouse—All pharmacokinetic (PK) studies were conducted according to protocols approved by the Institutional Animal Care and Use Committee at Genentech, Inc. Sprague Dawley rats were supplied by Charles River Laboratories (Hollister, Calif. USA). New Zealand White (NWZ) rabbits were supplied by Myrtle's Rabbitry (Thompson Station, Tenn. USA). BALB-c mice were supplied by Charles River (Hollister, Calif. USA).

Rats weighing between 279 to 314 g were given a 5 mg/kg body weight, IV bolus dose of AB.Fab variant 4D5-H, 4D5-H4, 4D5-H8, 4D5-H10 or 4D5-H1 1 (n=4 rats/group) via a cannula inserted in the femoral vein. At pre-dose, and over the course of 7 days post-dose, plasma was collected via a cannula inserted in the jugular vein.

Rabbits weighing between 2.9 to 3.5 kg were given a 0.5 mg/kg body weight, IV bolus dose of AB.Fab variant 4D5-H, 4D5-H4, 4D5-H8, 4D5-H1O, 4D5-H1 1 or 4D5-Fab (n=3 rabbits/group) via a catheter inserted in the marginal ear vein. At pre-dose and over the course of 21 days post-dose, serum was collected via an arterial catheter inserted in the contralateral ear.

Mice weighing between 17 and 20 g were given 5 mg/kg body weight IV bolus dose of AB.Fab variant 4D5-H, 4D5-H4, or 4D5-H8 (n=9 mice/group) via the tail vein. Over the course of 7 days serum was collected in 3 mice per time point by retro-orbital bleed or cardiac stick.

All serum and plasma samples were assayed for 4D5-Fab or AB.Fab variant concentrations using a HER2 Binding ELISA. Briefly, samples were added to microtiter wells coated with HER2ecd and incubated for 2 hours at room temperature. The wells were washed, and goat anti-huFab-HRP was added and incubated for 1 hour at room temperature. Unbound HRP was removed by washing and enzyme substrate was added to detect bound HRP. After 15 minutes, the reaction was quenched by the addition of 1 M phosphoric acid. The absorbance at 450 nm was read with a reference wavelength of 650 nm. Concentrations of AB.Fab were extrapolated by comparison to a standard curve of the dosed molecule.

For rats and rabbits, concentration versus time profiles for each animal were fit to a two compartment model using iterative re-weighting to estimate the pharmacokinetic parameters of Area Under the Concentration-Time Curve (AUC), Clearance (CL), Beta Half-Life (t_{1/2β}), Volume of Distribution of the Central Compartment (V₁), and Steady State Volume of Distribution (Vₛₛ) using WinNonlin® software (Version 3.2, Pharsight, Inc., Mountain View, Calif.) and group means were calculated.

In mice, a group mean serum concentration versus time profile was determined, producing one estimate for pharmacokinetic parameters for each AB.Fab dosing group. AB.Fab variants 4D5-H and 4D5-H4 were analyzed by a two compartment model as described above. AB.Fab variant 4D5-H8 was fit to a one compartment model and estimates for the PK parameters of AUC, CL, Half-Life (t_{1/2}) and V₁ were determined using WinNonlin® software (Pharsight, Inc., supra).

### RESULTS

Design, construction and purification—AB.Fab variants were engineered to possess a wide range of affinities for albumin as disclosed herein (see also, Dennis, M. S. et al. (2002), supra) . Relative in vivo stability of the AB.Fab variants was maintained by reducing the length of the albumin binding peptide rather than to alter its amino acid sequence (Table 12). In addition, it was discovered that the GGGS linker sequence used between the Fab and the peptide could be deleted without significantly affecting albumin binding.

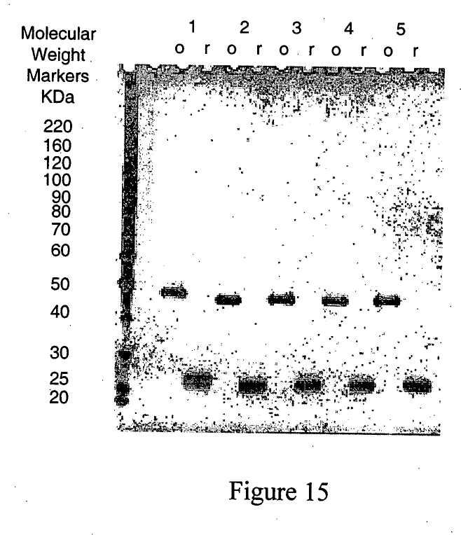

Despite their differing affinities for rabbit albumin, all of the AB.Fab variants could be rapidly and efficiently purified using a rabbit albumin affinity column as disclosed above in this Example. AB.Fab variants were essentially greater than 99 percent pure following the albumin affinity column. An additional cation exchange step was used to remove trace endotoxin and E. coli proteins to make the proteins suitable for in vivo studies. An SDS PAGE analysis of AB.Fab variants 4D5-H, 4D5-H4, 4D5-H8, 4D5-H10 and 4D5-H11 is shown in FIG. 15.

AB.Fab affinities for albumin—Several assays were explored in an effort to accurately determine the affinity of the AB.Fab variants for mouse, rat and rabbit albumin. Initially surface plasmon resonance was employed. Direct AB.Fab variant binding to immobilized albumin from various species resulted in inconsistent kinetic measurements for some of the weaker affinity variants (e.g. AB.Fab variants 4D5-H10 and 4D5-H1 1); however, the relative rank affinity of the variants could be determined as 4D5-H>4D5-H8>4D5-H4>4D5-H10>4D5-H11, with 4D5-H showing the highest affinity. This affinity ranking of the AB.Fab variants remained the same whether binding to mouse, rat or rabbit albumin.

As an alternative to surface plasmon resonance, an ELISA method was developed as disclosed herein that accurately determines the dissociation constant (Kd) of the AB.Fab variants to different species of albumin including, without limitation, albumin from mouse, rat, and rabbit. Initially we explored binding of the AB.Fab variants to immobilized albumin using a Direct Binding ELISA. The EC₅₀ for each AB.Fab and albumin combination is listed in Table 14. Estimates of EC₅₀ were made for AB.Fab variants 4D5-H10 and 4D5-H11 as a result of their lower signals.

TABLE 14
Assay comparison of the albumin affinities determined for each of
the AB.Fab variants with mouse, rat, rabbit and human albumin.*

| | Direct Binding ELISA | Solution Binding ELISA |
|---|---|---|
| | (EC₅₀, nM) | (Kd, nM) |
| Mouse Albumin | | |
| AB.Fab4D5-H | 0.070 ± 0.007 | 44 ± 1 |
| AB.Fab4D5-H4 | 0.077 ± 0.009 | 52 ± 7 |
| AB.Fab4D5-H8 | 0.043 ± 0.008 | 41 ± 4 |
| AB.Fab4D5-H10 | 14.52 ± 0.003 | 2500 ± 289 |
| AB.Fab4D5-H11 | >2000 | 1250 ± 99 |
| Rat Albumin | | |
| AB.Fab4D5-H | 0.065 ± 0.18 | 92 ± 5 |
| AB.Fab4D5-H4 | 0.075 ± 0.006 | 149 ± 23 |
| AB.Fab4D5-H8 | 0.045 ± 0.005 | 145 ± 24 |
| AB.Fab4D5-H10 | 8 ± 0.003 | 493 ± 81 |
| AB.Fab4D5-H11 | >1000 | 2429 ± 320 |
| Rabbit Albumin | | |
| AB.Fab4D5-H | 25 ± 2 | 36 ± 2 |
| AB.Fab4D5-H4 | 500 ± 50 | 444 ± 25 |
| AB.Fab4D5-H8 | 500 ± 50 | 247 ± 36 |
| AB.Fab4D5-H10 | >2000 | 1065 ± 87 |
| AB.Fab4D5-H11 | >2000 | 1110 ± 32 |
| Human Albumin | | |
| AB.Fab4D5-H | 608 ± 41 | 556 ± 54 |

*Values represent the average and standard deviations from at least 3 determinations.

AB.Fab Binding to immobilized albumin may differ from binding to albumin in solution (i.e. in plasma) due, for example, to potential artifacts arising from the absorption of albumin on plastic, thereby distorting the true binding affinities of the Ab.Fab variants for albumin in solution. To eliminate this possibility, a 2-step. ELISA approach was developed herein. Similar methods were described by Friguet et. al. (Friguet, B. et al. (1985) J. Immunol. Methods 77, 305-319) and also used for determining the K_{d} of humanized antibodies to HER2 (Carter, P et al.. (1992) Bio/Technology 10, 163-167). This assay established a solution phase binding equilibrium followed by detection of unbound (free) AB.Fab using the Direct Binding ELISA (disclosed herein, above).

During use of the Solution Binding ELISA to generate the solution phase binding equilibrium, it is preferred that the concentration of AB.Fab variant is as low as possible while still providing a sufficient signal to measure free AB.Fab. The minimum concentration of each AB.Fab variant used with each species of albumin was determined by titrating the AB.Fab variant in the Direct Binding ELISA using the corresponding immobilized albumin. This minimum concentration of AB.Fab was then titrated with soluble albumin in the Solution Binding ELISA. The minimum concentration of each AB.Fab variant and the initial albumin concentration used are listed in Table 13. The concentration of unbound (free) AB.Fab variant was then determined utilizing the Direct Binding ELISA in which the corresponding albumin was immobilized. In order to determine the K_{d} value using Scatchard Analysis (Scatchard, G. (1947) Ann. N.Y. Acad. Sci. 51, 660), the equilibrium between the AB.Fab variant and albumin in solution was reached prior to the determination of un-bound AB.Fab variant.

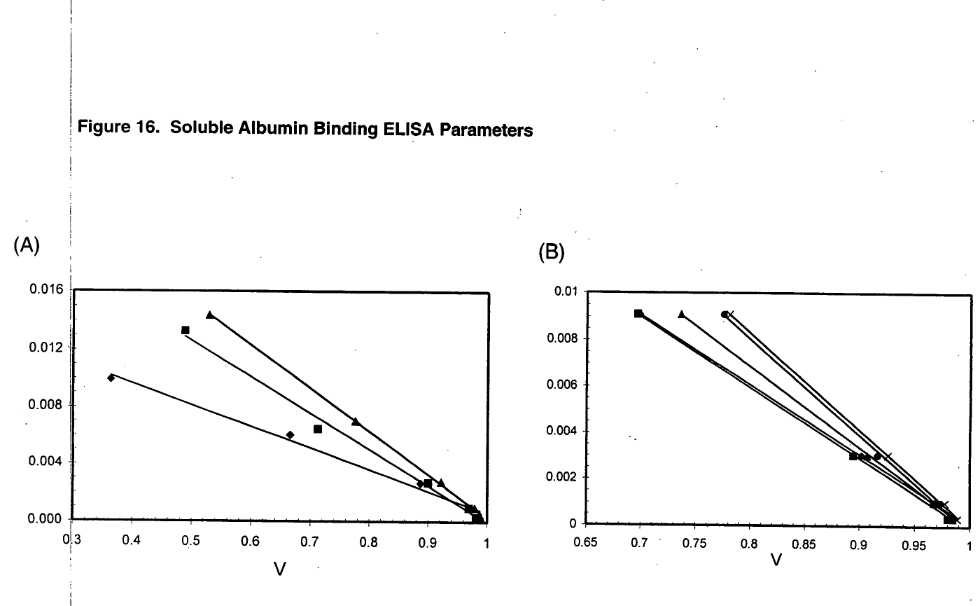

To verify that the AB.Fab-albumin mixture had reached equilibrium, AB.Fab 4D5-H was incubated with rabbit albumin at 1, 2 and 16 hours before the mixtures were assayed in the Direct Binding ELISA. Equilibrium was essentially reached after a 2 hour incubation (FIG. 16A). The optimal time needed to capture unbound (free) AB.Fab 4D5-H to immobilized albumin was determined by incubating the AB.Fab-albumin mixture with immobilized albumin for 15, 30, 45, 60 and 120 minutes. The minimum amount of time required to bind the free Fab-H was 15 minutes; however, for convenience, 30 minutes was ultimately used (FIG. 16B).

Under these experimental conditions, the capture of free AB.Fab in the Direct Binding ELISA did not significantly shift the AB.Fab and albumin equilibrium, so the fraction of bound AB.Fab variant (v) was related to the signal measured in the Direct Binding ELISA. Since the concentration of total albumin was 10 to 1000 fold higher than the concentration AB.Fab variant, the concentration of free albumin (a) approximated the total albumin concentration. Thus, the K_{d} was determined by plotting the fraction of bound (v) vs. v/a (Scatchard, G. (1947), supra). A comparison of the AB.Fab variant affinities for mouse, rat and rabbit albumin determined by these various assay methods is summarized in Table 14.

The AB.Fab variants present an even distribution of affinities for rabbit albumin over a 30-fold range from 36 to 1110 nM. Similarly in rat, the affinities ranged over 27-fold from 92 to approximately 2500 nM. In mouse, however the distribution was different with 4D5-H, 4D5-H4 and 4D5-H8 having very similar affinities and 4D5-H10 and 4D5-H11 displaying very weak affinities for mouse albumin.

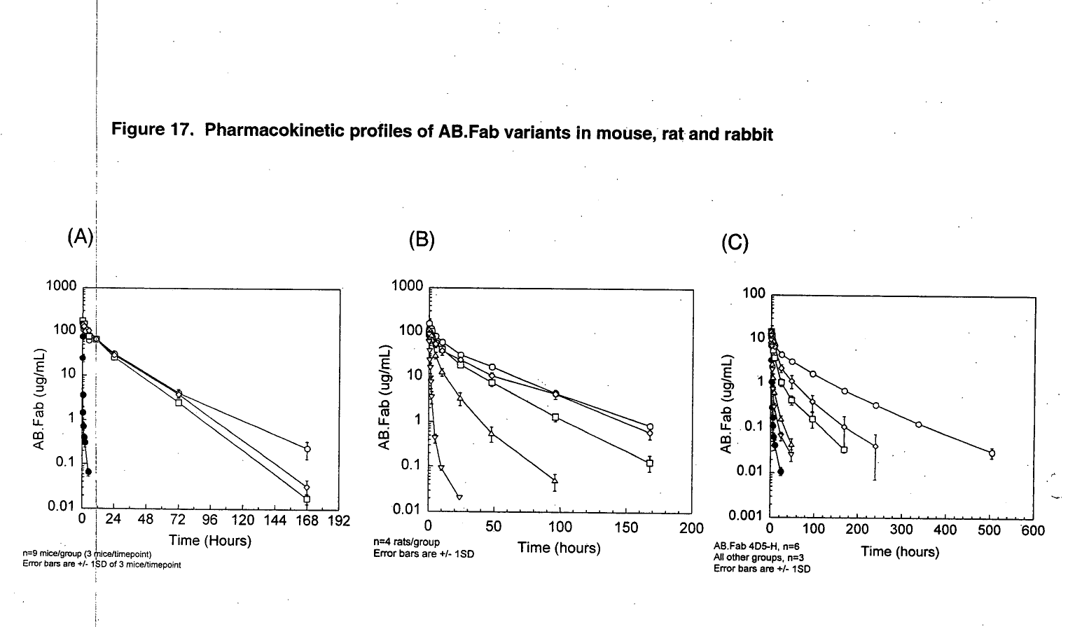

Pharmacokinetics of AB.Fab variants in rat, rabbit, and mouse—To explore the role of albumin binding affinity on the ability of albumin to extend the half-life of an AB.Fab, the PK parameters of the AB.Fab variants in mouse, rat and rabbit were investigated. In rats and rabbits, an affinity dependent increase in exposure (AUC) was observed. Group mean PK parameters are listed in Table 15. PK profiles are plotted in FIG. 17. In both rats and rabbits, the AB.Fab variants displayed biphasic elimination.

TABLE 15
Summary of AB.Fab pharmacokinetic parameters in mouse, rat and rabbit

| | | | Kd for | Pharmacokinetic Parameter Estimates | | | | |
|---|---|---|---|---|---|---|---|---|
| | Dosing | Number of | Albumin | AUC | CL | t_{1/2β} | V₁ | Vₛₛ |
| Species | Material | Animals | (nM) | (hr × ug/mL) | (mL/hr/kg) | (hr) | (mL/kg) | (mL/kg) |
| Mouse | 4D5Fab | 9 | —^{a} | 20.9 | 239 | 1.28 | 36.8 | 68.6 |
| | AB.Fab4D5-H | 9 | 44 | 2390 | 2.09 | 19.7 | 30.1 | 56.3 |
| | AB.Fab4D5-H4 | 9 | 52 | 2070 | 2.41 | 13.5 | 28.4 | 44.4 |
| | AB.Fab4D5-H8 | 9 | 41 | 2610 | 1.91 | 14.0^{b} | N/A^{b} | 38.7 |
| Rat | AB.Fab4D5-H | 4 | 92 | 2880 ± 466 | 1.50 ± 0.220 | 26.9 ± 3.11 | 27.3 ± 9.54 | 50.8 ± 9.02 |
| | AB.Fab4D5-H4 | 4 | 149 | 1450 ± 146 | 3.24 ± 0.352 | 20.7 ± 1.07 | 52.2 ± 4.21 | 76.0 ± 6.56 |
| | AB.Fab4D5-H8 | 4 | 145 | 1890 ± 294 | 2.44 ± 0.453 | 28.0 ± 1.88 | 45.8 ± 8.11 | 85.9 ± 15.4 |
| | AB.Fab4D5-H10 | 4 | 493 | 541 ± 77.9 | 9.21 ± 1.24 | 10.9 ± 2.86 | 44.6 ± 2.04 | 75.3 ± 9.20 |
| | AB.Fab4D5-H11 | 4 | 2500 | 57.0 ± 7.39 | 80.2 ± 11.8 | 4.21 ± 0.151 | 48.7 ± 2.25 | 81.6 ± 5.76 |
| Rabbit | 4D5Fab | 3 | —^{a} | 8.75 ± 1.05 | 60.8 ± 7.37 | 5.98 ± 0.209 | 51.7 ± 1.07 | 122 ± 5.57 |
| | AB.Fab4D5-H | 6 | 36 | 514 ± 48.9 | 0.841 ± 0.053 | 68.5 ± 5.58 | 38.0 ± 2.76 | 70.0 ± 6.59 |
| | AB.Fab4D5-H4 | 3 | 444 | 127 ± 16.5 | 3.58 ± 0.496 | 28.3 ± 2.88 | 30.5 ± 1.67 | 82.2 ± 10.2 |
| | AB.Fab4D5-H8 | 3 | 247 | 232 ± 62.7 | 1.99 ± 0.477 | 36.2 ± 7.13 | 29.6 ± 3.32 | 65.9 ± 7.10 |
| | AB.Fab4D5-H10 | 3 | 1065 | 33.7 ± 3.32 | 13.5 ± 1.40 | 12.0 ± 2.21 | 36.8 ± 4.12 | 98.7 ± 14.1 |
| | AB.Fab4D5-H11 | 3 | 1110 | 24.6 ± 2.74 | 20.6 ± 2.24 | 11.9 ± 2.61 | 40.4 ± 4.38 | 101 ± 31.7 |

AUC: Area Under the Concentration-Time Curve, CL: Clearance, t_{1/2β}: Beta Half-Life, V₁: Volume of Distribution of the Central Compartment, Vₛₛ: Steady State Volume of Distribution
^{a}No specific binding affinity for albumin.
^{b}For AB.Fab 4D5-H8 in mouse, elimination fit to one-compartment model, providing one estimate for half life and volume.
N/A Not applicable

In rats, a 27-fold increase in albumin binding affinity resulted in a 50-fold increase in exposure. AUC ranged from 57.0±7.39 to 2880±466 h×ug/mL. In rabbits, a 30-fold increase in albumin binding affinity among the AB.Fab variants resulted in a 20-fold increase in exposure. AUC among the AB.Fab variants ranged from 24.6±2.74 to 514.5±48.9 h×ug/mL. AUC for the wild type 4D5 Fab was 8.75±1.05 h×ug/mL.

Increased albumin binding affinity of the AB.Fab variants resulted in decreased clearance in rats and rabbits (Table 15). In rats, clearance decreased 53-fold, with clearance ranging from 80.2±11.8 to 1.50±0.220 mL/h/kg. In rabbits, clearance of the AB.Fab variants ranged from 20.6±2.24 to 0.841±0.053 mL/h/kg, approximately a 24-fold decrease. By comparison, the clearance of wild type 4D5 Fab (60.8 mL/h/kg), with no specific binding affinity for albumin, was 3 to 73-fold faster in rabbits than any of the AB.Fab variants. The pharmacokinetic profiles of the AB.Fab variants in rat and rabbit are shown in FIG. 17.

In both rats and rabbits, terminal half-life increased approximately 6-fold as a result of increased affinity among the AB.Fab variants. Terminal half-life of the AB.Fab variants ranged from 4.21±0.151 to 26.9±3.11 h in rats and 11.9±2.61 to 67.6±3.98 h in rabbits. In both rats and rabbits, the volume of distribution of the central compartment (V₁) for all AB.Fab variants approximated serum volume.

In summary, there was a direct correlation between Ab.Fab variants with a high affinity for albumin and a slower clearance and longer half-life in either rat or rabbit. In mouse, the PK of AB.Fab variants 4D5-H, 4D5-H4 and 4D5-H8 displayed similar clearance consistent with their similar affinities for mouse albumin (Table 14). Further, in mouse and rabbit, all AB.Fab variants tested had a slower clearance than the wild-type Fab-4D5.

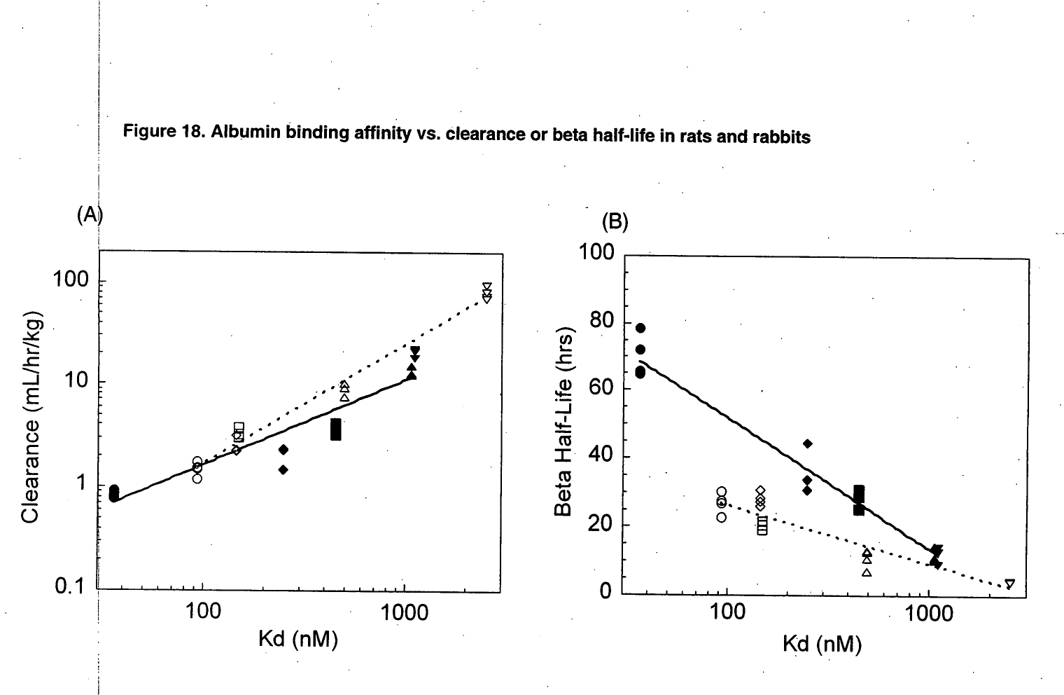

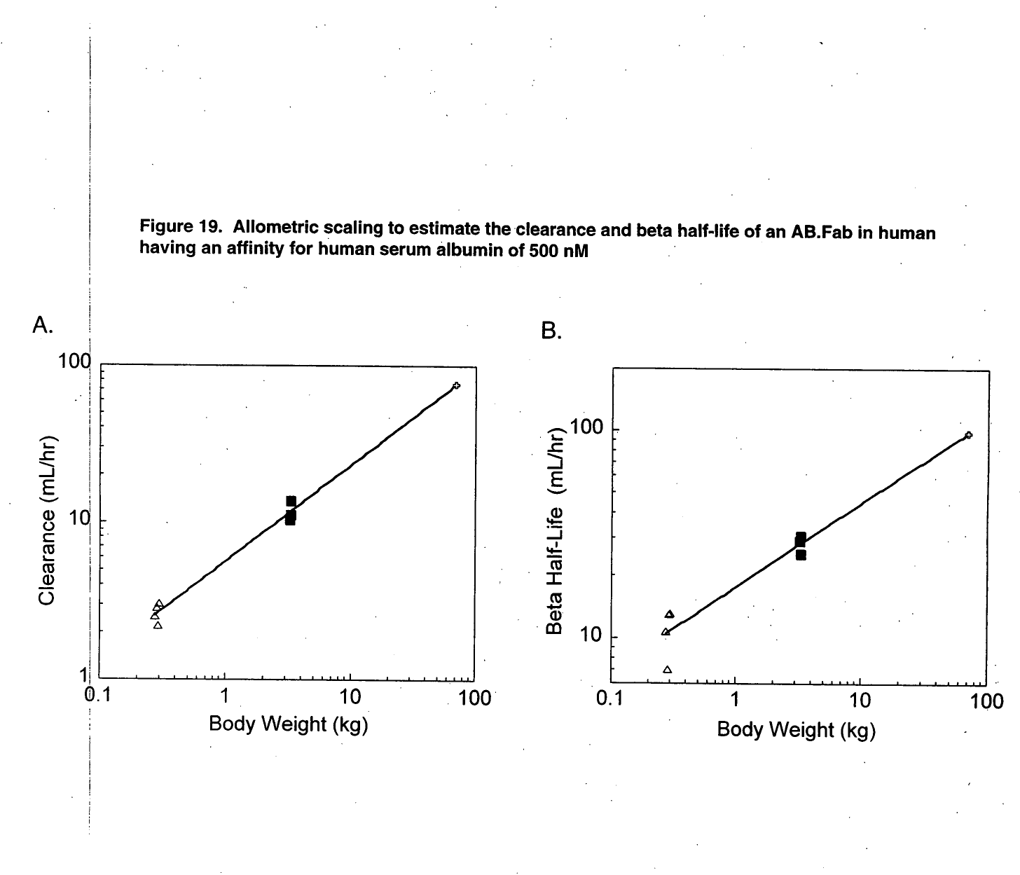

Correlation between albumin binding affinity and clearance—The correlation between albumin binding and beta half-life is illustrated for both rabbits and rats in FIG. 18. The separation of the curves for rabbits and rats illustrates that it is preferred that AB.Fab variants with similar albumin binding are compared for appropriate allometric scaling. In rabbits Ab.Fab4D5-H4 has binding affinity of 444 nM and in rats Ab.Fab4D5-H10 has binding affinity of 493 nM similar to the affinity of AB.Fab4D5-H in human of 556 nM (Table 14). Allometric scaling of the PK parameter estimates of clearance and beta half life is shown in FIG. 19 and can be described using the following equations:
Clearance (mL/h)=5.65*Body Weight (kg)^{0.611}
and
Beta Half Life (h)=17.5* Body Weight (kg)^{0.406}.
Using the equations above, the predicted clearance and beta half-life of an AB.Fab with a binding affinity of approximately 500 nM, in humans is approximately 76 mL/h (approximately 1 mL/h/kg) and 4 days respectively. Mouse data were not included in the allometric scaling estimate of clearance and beta half life in humans because the binding affinities of the AB.Fab variants for mouse albumin were outside the rat and rabbit range of affinities utilized for making the estimate.
Discussion

Covalent association with albumin has been achieved through the genetic fusion of rapidly cleared proteins to albumin (Yeh, P. et al. (1992) Proc. Natl. Acad. Sci. USA 89, 1904-1908; Syed, S. et al. (1997) Blood 89, 3243-3252; Sung, C. et al. (2003) J. Interferon & Cytokine Res 23, 25-36; Marques, J. A. et al. (2001) Thromb Haemost 86, 902-908; Smith, B. J. (2001) Bioconjugate Chem. 12, 750-756), through non-specific chemical modification to attach proteins (30) or small molecules (Stehle, G. et al. (1997) Anti-Cancer Drugs 8, 677-685), and through specific modification using the reactive free cysteine in albumin (Smith, B. J. et al. (2001) Bioconjugate Chem. 12, 750-756; Kratz, F. et al. (2000) J. Med. Chem. 43, 1253-1256). Unlike these covalent fusions to albumin, the non-covalent association of the AB.Fab variants with albumin allows their clearance to be modified as disclosed herein. The fraction of free unbound AB.Fab in serum can be calculated using the affinity of each variant for albumin, the reported serum concentration of albumin, 600 μM (Peters, T., Jr. (1996) All about albumin, Academic Press, Inc., San Diego, page 256), and the following equation:

$${Fraction}_{Free} = 1 - \frac{[ albumin ]}{[ albumin ] + ( {Kd}_{AB . Fab variant} )}$$

From this perspective, it appears that a very small difference in the fraction of AB.Fab that is unbound by albumin in vivo will have a profound effect on its rate of clearance. Thus the affinities of these AB.Fab variants for albumin lie in a range where an increase or decrease in association has a measurable effect on clearance and half-life. The curves in FIG. 18 indicate that further increases in albumin binding affinity could lead to further increases in half-life since a plateau was not in the affinity range plotted, although at some point, other clearance mechanisms may have an effect.

Albumin binding has been employed previously as a strategy for reducing the clearance of fatty acid acylated insulins (Markussen, J. et al. (1996) Diabetologia 39, 281-288). A direct correlation between albumin binding and clearance in pigs was observed for nine derivatives over an affinity range from 4 to 70 μM. The AB.Fab variants presented here have affinities for albumin that are 10-fold higher and demonstrate a continued and more dramatic reduction in clearance. The AB.Fab association with albumin does not impair the interaction of the Fab with antigen nor does it compete with any of the physiological known ligands that are carried by albumin in vivo (Dennis, M. S. et al. (2002) J. Biol. Chem. 277, 35035-35043). The combined affinity range observed to impact the half-lives of the AB.Fab variants disclosed herein and the fatty acid acylated insulins (Markussen, J. et al. (1996), supra) is similar to the affinity range of many physiologically relevant molecules that are carried by albumin. For example, many organic anions have affinities of 1-100 μM and long chain fatty acids bind to albumin in the 100 nM range (Peters, T., Jr. (1996) All about albumin, Academic Press, Inc., San Diego, page 77). The affinity of these molecules for albumin as well, likely plays a role in their rate of clearance.

The prolonged half-life and reduced clearance of two different Fab fragments are disclosed herein to occur through their association with albumin by way of an albumin-binding peptide. In one embodiment of the invention, an albumin-binding peptide sequence was fused to the carboxyl terminus of the light chain of an anti-tissue factor Fab. In another embodiment, an albumin-binding peptide sequence was fused to the carboxyl terminus of the light chain of an anti-HER2 Fab. Similar pharmacokinetic parameters were observed for both sets of AB.Fab variants. The present disclosure demonstrates that the enhanced pharmacokinetics of an albumin-binding Fab is a direct function of its affinity for albumin. Further, by utilizing AB.Fab variants with varied affinities for albumin, the elimination half-life for AB.Fab4D5-H in humans was determined.

Albumin binding is a common strategy for reducing the clearance of small molecule pharmaceuticals. This information could be useful in the design of such drugs where, unlike the AB.Fab, the interplay between achieving a prolonged half-life as a result of albumin binding is balanced against a potential loss in function as albumin binding of the small molecule precludes it from binding to its intended target. Once establishing the concentration of free drug required for efficacy, the balance between this concentration and its potential half-life as a function of albumin binding might be estimated from this data.

By extending the half-life of a Fab, an AB.Fab may also have utility in tumor targeting where it has been observed that size and half-life of the targeting agent can have a dramatic affect on tumor delivery and retention (Wu, A. M., and Yazaki, P. J. (2000) Quart. J. Nucl. Med. 44, 268-283; Hu, S. et al. (1996) Cancer Res. 56, 3055-3061). The ability to fine tune the pharmacokinetics of an AB.Fab as disclosed herein is useful in identifying the ideal properties required for optimum delivery of Fab to its therapeutic target.

### EXAMPLE 6

### AB.Fab enhances Tumor Tissue Penetration

The following example demonstrates that fusion of an albumin-binding peptide to an antibody fragment according to the invention enhances penetration of the AB.Fab into tumor tissue. This is illustrated in the following experiment in which AB.Fab4D5H, disclosed herein, was shown to penetrate breast tumor tissue faster and to a greater extent (e.g. greater penentrated cell area relative to total cell area) than Fab4D5 alone or Herceptin® (trastuzumab, Genentech, Inc., South San Francisco, Calif.) alone. While the following example discloses enhanced AB.Fab penetration into breast tumor tissue expressing HER2, it can be readily appreciated that tissue penetration of a Fab can be enhanced according to the invention by fusing an albumin binding peptide to a Fab which binds a target molecule expressed in a tissue or on a tissue cell.

Herceptin® antibody (huMAb4D5, see U.S. Pat. No. 5,821,337, issued Oct. 13, 1998, incorporated herein by reference) is a humanized monoclonal antibody specific for human epidermal growth factor receptor 2 (HER2). HER2 is overexpressed in approximately 30% of breast cancer and Herceptin® antibody provides an effective treatment for HER2-positive breast cancer. Slow clearance of Herceptin® antibody maintains a relatively high concentration of the antibody drug in tumor tissue. The relatively large size of antibodies can, however, slow the rate of diffusion of the antibody in tissues, limit vascular permeability, cause heterogeneous distribution within tissue, and limit penetration into tissue. Small antibody fragments (such as, for example, Fab4D5 derived from Herceptin® antibody) increase vascular permeability, diffusion, distribution and penetration into tumors due to its smaller molecular size. Smaller size, however, increases plasma clearance and can cause accumulation in the kidney with reduced deposition in tumor tissue. As disclosed herein, these possible limitations are avoided by the invention in which an albumin binding peptide is fused to Fab4D5 (Kelley, R. F., et al., Biochemistry 31:5434-5441 (1992) and Dennis, M. et al., J. Biol. Chem. 277:35035-35043 (2002)) to generate an AB.Fab having a smaller hydrodynamic radius than an antibody, but providing increased plasma half-life. The AB.Fab of the invention is shown in the following example to have increased penetration in tumor tissue compared to the full length Herceptin® antibody and reduced renal clearance relative to Fab4D5 lacking the albumin binding peptide.

Albumin-binding Fab4D5-H (AB.Fab4D5-H or AB.Fab, albumin binding peptide SEQ ID NO:401) derived from Herceptin® antibody is a bi-functional molecule that can simultaneously bind albumin (via an albumin binding peptide) and the tumor antigen HER2 (erbB2) (via the Fab4D5 moiety). Preparation of AB.Fab4D5-H is described herein above in Example 3. The small antibody fragment may increase its ability to penetrate into tumors compared to the full length IgG Herceptin® antibody while its association with albumin substantially reduces clearance relative to Fab4D5 (Fab). This study compared the uptake and distribution of Herceptin® full length antibody, AB.Fab (albumin binding peptide-Fab4D5 fusion) and Fab (Fab4D5) using FITC conjugates in a dorsal skin window model. After anesthesia with ketamine/zylazine, two symmetrical titanium frames were used to sandwich the extended double layer of skin in athymic nude mice. One layer of skin was removed in a circular area approx 15 mm in diameter, and the remaining layer was covered with a glass overslip incorporated into one of the frames. Two days later, a piece (1 mm in diameter) of HER2-F2-1282 tumor was implanted into the center of the window. The MMTV-HER2-F2-1282 mammary tumor was from a HER2 transgenic mouse whose HER2 expression is targeted to the mammary gland using the MMTV promoter (see U.S. Pat. Application Nos. 20020001587, filed Mar. 16, 2001, and 20020035736, filed Mar. 16, 2001, incorporated herein by reference). When tumors reached the desired size, the mice were randomized to receive 10 mg/kg FITC-Herceptin, 20 mg/kg FITC-Fab, or 20 mg/kg FITC-AB.Fab (n=3 in each group) by i.v. injection. Tumors were observed and recorded using a confocal laser scanning microscope equipped with an intensified CCD camera at 15 and 45 minutes, 2, 6, and 24 hours, 2, 3, 4, and 5 days. The time-course study indicated that the uptake of fluorescence in tumor cells was maximal at 6 hours for FITC-Fab or 24 hours for FITC-AB.Fab and FITC- Herceptin® antibody after injection (see Table 16).

TABLE 16
Uptake of FITC-Herceptin ®, FITC-Fab4D5,
and FITC-Ab.Fab4D5-H In Breast Tumor Tissue

| Time | Herceptin ® | Fab4D5 | | Ab.Fab4D5-H |
|---|---|---|---|---|
| Initial Fluorescence | 6 hours | 45 | minutes | 2 hours |
| Maximum Fluorescence | 1 day | 2-6 | hours | 1 day |
| Sustained Fluorescence | 5 days | 6 | hours | 5 days |

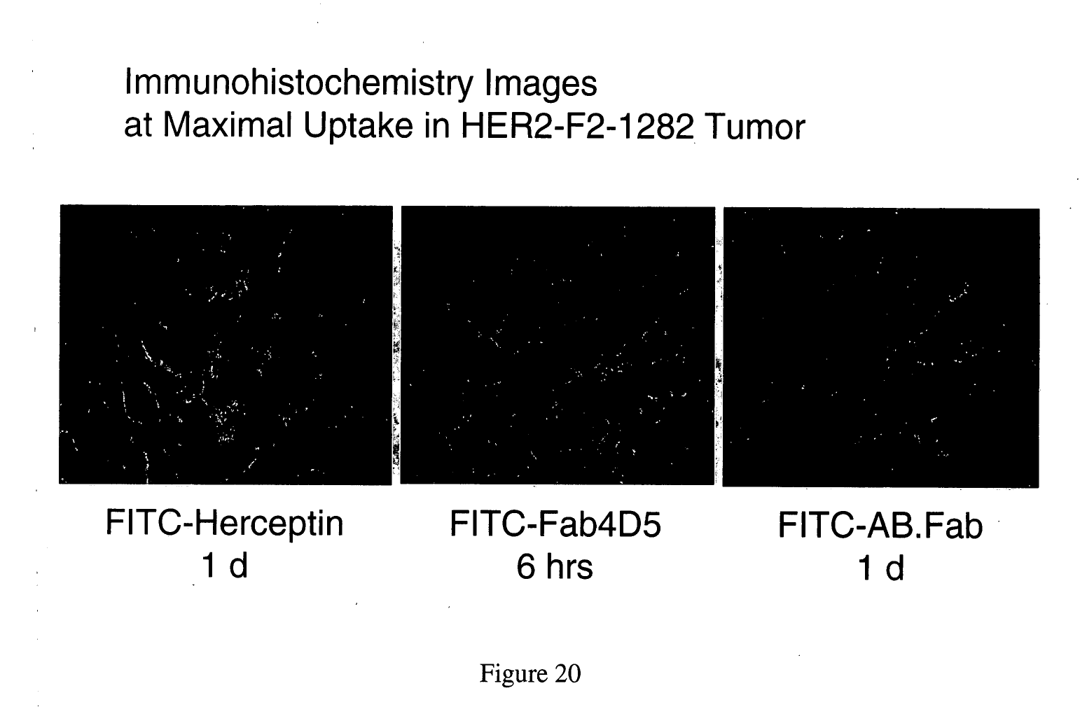

The tumors contacted with FITC-Herceptin, FITC-Fab, or FITC-AB.Fab were imaged by intavital microscopy, including during the time of maximal uptake according to the above experiment. Following visualization, tumors were excised, embedded in OCT (optimal cutting temperature) compound and frozen at −80° C. for immunohisochemistry studies (n=4-5 in each group). Tissue slides were mounted with Vectashied® mounting media (Vector Laboratories, Burlingame, Calif.) and contacted with rat anti-mouse CD31 antibody and Cy3-goat anti-rat IgG antibody conjugate (available from, for example, Research Diagnostics, Inc., Concord, Mass., USA and Millegen, Labege, France, respectively) and DAPI nuclear stain. Tissue stained according to this procedure is shown in FIG. 20. The figure shows that during the time of maximum uptake, the HER2-expressing breast tumor cells were stained by FITC-conjugated Herceptin®, Fab4D5 and Ab.Fab4D5H (green spots). Cell nuclei are stained blue by DAPI, and vasculature was stained red by binding of the anti-CD31 antibody and secondary binding of the Cy3-antibody conjugate.

Using intravital microscopy, tumor vasculature was visualized at 15 min after injection for each of FITC-conjugated Herceptin®, Fab4D5 and Ab.Fab4D5H. Penetration into tumor cells was initiated at 45 min by Fab, 2 hours by AB.Fab, and 6 hours by Herceptin®, while tumor deposition was sustained for only 6 hours by Fab but up to 5 days by AB.Fab or Herceptin®. Both intravital microscopic and histological imaging showed that Herceptin® penetrated only the outer-layers of HER2-F2-1282 tumor cells, while the tumor tissue area penetrated by both Fab and AB.Fab was increased compared to that of Herceptin®.

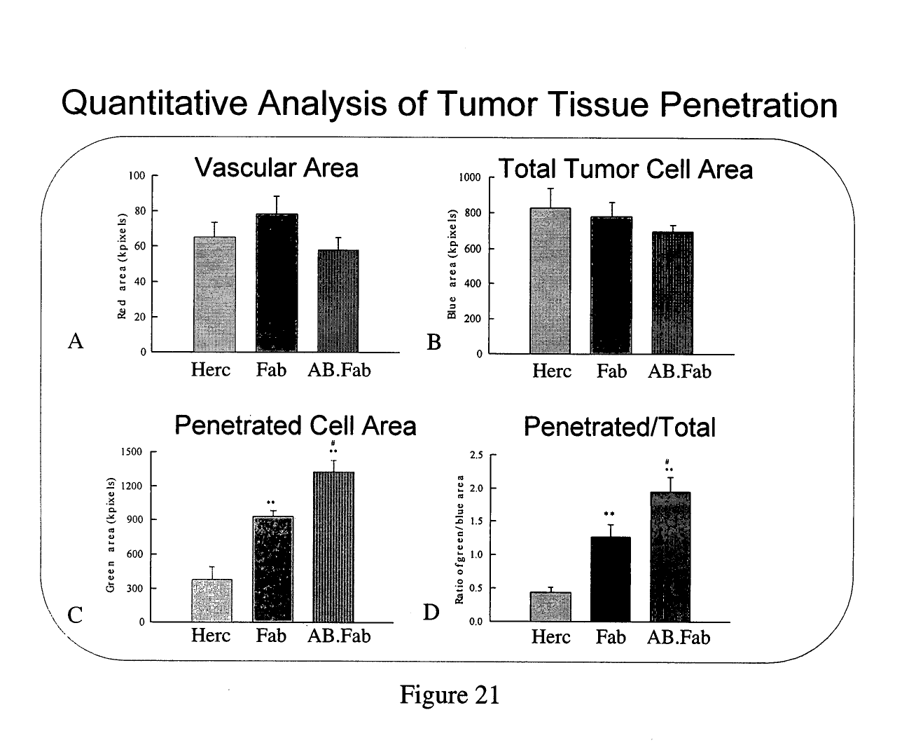

To determine the amount of tumor area penetrated by FITC-conjugated Herceptin®, Fab4D5 and AB.Fab4D5-H at the time of maximum uptake, staining of the histological sections was quantitated using ImageJ software (a public domain Java image processing program developed by the National Institutes of Health for the Macintosh, http://rsb.info.nih.gov/ij/docs/index.html). The results are shown in FIG. 21A-21D. The penetrated cell area (FIG. 21C) was compared to the total tumor cell area (FIG. 21B) to provide the ratio of penetrated area to total area (FIG. 21D). Based on these results, it can be seen from FIG. 21C that the tumor tissue area penetrated by Fab or AB.Fab was significantly larger than that of Herceptin® (P<0.01), while the area penetrated by AB.Fab was larger than that of Fab4D5 (P<0.05). Similarly, the ratio of penetrated area to total cell area was significantly higher for Fab or AB.Fab compared to Herceptin® (P<0.01) and the ratio was even greater for AB.Fab than Fab (P<0.05) (see FIG. 21D). These data demonstrate that compared to Herceptin®, AB.Fab exhibits rapid targeting and improved tumor cell penetration while retaining sustained tumor deposition, thereby demonstrating its usefulness in imaging and therapy.

All publications cited herein are expressly incorporated by reference in their entirety. The instant invention is shown and described herein in what is considered to the the most practical, and the preferred embodiments. It is recognized, however, that departures may be made therefrom which are within the scope of the invention, and that obvious modifications will occur to one skilled in the art upon reading this disclosure.

We claim:

1. A method for modulating tissue distribution of a peptide molecule, comprising administering to the tissue a conjugate molecule comprising a peptide ligand domain and an active domain, wherein the peptide ligand domain comprises a serum albumin binding peptide and the active domain comprises the peptide molecule; and wherein said administering of the conjugate molecule results in tissue distribution of the peptide molecule that differs from that obtained on administration of the active domain alone.

2. The method of claim 1, wherein said modulating comprises enhancing the rate of tissue uptake of the conjugated peptide molecule.

3. The method of claim 1, wherein said modulating comprises enhancing the rate of diffusion of the conjugated peptide molecule in the tissue.

4. The method of claim 1, wherein said modulating comprises enhancing the distribution of the conjugated peptide molecule through the tissue.

5. The method of claim 1, wherein said modulating comprises matching the rate of tissue uptake of the conjugated peptide molecule to the rate of internalization of one or more tissue receptors.

6. The method of claim 1, wherein said modulating comprises enhancing tissue penetration of the conjugate peptide molecule relative to a peptide molecule.

7. The method of claim 6, wherein the tissue is tumor tissue.

8. The method of claim 7, wherein the tissue is breast tumor tissue.

9. The method of claim 8, wherein the conjugated peptide molecule comprises an anti-HER2 Fab fragment.

10. The method of claim 5, wherein the conjugate peptide molecule comprises a tracer or label.

11. The method of claim 1, wherein said peptide is an antibody or antibody fragment that binds a receptor expressed by the tissue.

12. The method of claim 11, wherein said antibody or antibody fragment is an anti-HER2 antibody, an anti-CD20 antibody, an anti-EGFR antibody, an anti-VEGF antibody, an anti-CD40 antibody, or a frament thereof.

13. The method of claim 1, wherein said serum albumin binding peptide comprises the following amino acid sequence: Xaaᵢ-Cys-Xaaⱼ-Cys-Xaaₖ, where the sum of i, j, and k is about 25 or less.

14. The method of claim 13, wherein the sum is about 18 or less.

15. The method of claim 14, wherein the sum is about 11 or less.

16. The method of claim 1, wherein the affinity of the serum albumin binding peptide for albumin is characterized by an equilibrium dissociation constant (K_{d}) that is about 500 nM or less.

17. The method of claim 16, wherein said K_{d} is about 100 nM or less.

18. The method of claim 16, wherein said K_{d} is about 50 nM or less.

19. The method of claim 16, wherein said K_{d} is about 10 nM or less.

20. The method of claim 1, wherein said conjugate molecule comprises a linker moiety disposed between said peptide ligand domain and said active domain.

21. The method of claim 20, wherein said linker moiety comprises the amino acid sequence: GGGS.

22. The method of claim 1, wherein said active domain comprises a therapeutic or diagnostic substance.

23. The method of claim 22, wherein said substance is a protein.

24. The method of claim 23, wherein said protein is an antibody or antibody fragment.

25. The method of claim 24, wherein said protein is a Fab, F(ab′)₂, or scFv fragment.

26. The method of claim 22, wherein said substance comprises a tracer or label.

27. The method of claim 1, wherein said serum albumin binding peptide comprises the core amino acid sequence: D X C L P X W G C L W (SEQ ID NO:423), where X is any amino acid.

28. The method of claim 27, wherein said serum albumin binding peptide comprises the core amino acid sequence: X₄ D X C L P X W G C L W X₃ (SEQ ID NO: 156), where X is any amino acid.

29. The method of claim 28, wherein said serum albumin binding peptide comprises the core amino acid sequence: X₅ D X C L P X W G C L W X₄ (SEQ ID NO: 155), where X is any amino acid.

30. The method of claim 27, wherein said peptide comprises the amino acid sequence: DICLPRWGCLW (SEQ ID NO:8).

31. The method of claim 30, wherein said peptide comprises the amino acid sequence: X₄ D I C L P R W G C L W X₃ (SEQ ID NO:424), where X is any amino acid.

32. The method of claim 31, wherein said peptide comprises the amino acid sequence: X₅ D I C L P R W G C L W X₄ (SEQ ID NO:425), where X is any amino acid.

33. The method of claim 1, wherein said serum albumin binding peptides comprises the amino acid sequence of SEQ ID NO: 7, 8, 9, 10, 11, 12, 13, 14, 15, 16, 17, 18, 19, or 20.

34. The method of claim 1, wherein said serum albumin binding peptide binds to two or more species of albumin.

35. The method of claim 27, wherein said serum albumin binding peptide bind to human albumin.

36. The method of claim 1, wherein said active domain comprises an antibody fragment, and wherein said binding ligand domain is fused to the N- or C-terminal region of a variable heavy or variable light chain.

37. The method of claim 36, wherein said antibody fragment comprises a Fab, F(ab)2, scFv, V_{H}, V_{L}, or diabody antibody binding fragment.

38. The method of claim 1, wherein said administering is administering to a patient via injection, inhalation, internasal, or oral methods.

39. The method of claim 1, wherein said conjugate molecule is admixed with a pharmaceutical carrier.

40. The method of claim 1, wherein said tissue is tumor tissue, and wherein said administering is administering to a patient a therapeutically effective amount.

41. The method of claim 1, wherein said tissue is tumor tissue, and wherein said administering is administering to a patient a diagnostically effective amount.
